(19) United States
(12) Patent Application Publication
(10) Pub. No.: US 2009/0275972 A1
(43) Pub. Date: Nov. 5, 2009

(54) MINIMALLY-INVASIVE METHODS FOR IMPLANTING OBESITY TREATMENT DEVICES

(76) Inventors: Shuji Uemura, San Francisco, CA (US); Brian K. Shiu, Sunnyvale, CA (US); Pablo G. Acosta, Newark, CA (US); Theodore M. Bender, Oakland, CA (US); Matthew B. Newell, Portola Valley, CA (US); Dane A. Johnson, San Francisco, CA (US); Beverly Huss, Menlo Park, CA (US); Joshua Makower, Los Altos, CA (US); Crystine M. Lee, San Rafael, CA (US); Timothy A. Limon, Cupertino, CA (US)

Correspondence Address: LAW OFFICE OF ALAN W. CANNON, 942 MESA OAK COURT, SUNNYVALE, CA, 94086, US

(21) Appl. No.: 12/473,881

(22) Filed: May 28, 2009

Related U.S. Application Data
(63) Continuation-in-part of application No. 11/716,985, filed on Mar. 10, 2007.
Application No. 11/716,985 is a continuation-in-part of application No. 11/716,986, filed on Mar. 10, 2007.
Application No. 11/716,986 is a continuation-in-part of application No. 11/407,701, filed on Apr. 19, 2006.
(60) Provisional application No. 61/130,244, filed on May 28, 2008.

(51) Int. Cl. A61M 29/00

(52) U.S. Cl. 606/192

(57) ABSTRACT

Tools, devices and methods for treating a patient. An incision or puncture is made though the patient's skin and a delivery tract is made through an opening formed by the incision or puncture, subcutaneous fat and fascia, far into the patient's abdominal cavity. A guide member is inserted through the delivery tract and a distal end portion of the guide member is positioned at a target location within the abdominal cavity. An implantable device is delivered with the aid of the guide member, and the implantable device is attached to at least one internal body structure. A fillable member of the implantable device is at least partially filled with fluid. The guide member is removed and an adjustment member is attached to a conduit in fluid communication with the fillable member. The attachment member is anchored to an internal body structure and the incision or puncture is closed.

### CROSS-REFERENCE

This application is a continuation-in-part application of co-pending application Ser. No. 11/716,985, filed Mar. 10, 2007 to which application we claim priority and which application is incorporated herein, in its entirety, by reference thereto.

This application is a continuation-in-part application of co-pending application Ser. No. 11/716,986, filed Mar. 10, 2007, to which application we claim priority and which application is incorporated herein, in its entirety, by reference thereto.

This application is a continuation-in-part application of co-pending application Ser. No. 11/407,701, filed Apr. 19, 2006 to which application we claim priority and which application is incorporated herein, in its entirety, by reference thereto.

This application claims the benefit of U.S. Provisional Application No. 61/130,244, filed May 28, 2008, which application is hereby incorporated herein, in its entirety, by reference thereto.

This application also hereby incorporates herein by reference thereto, in their entireties, co-pending application Ser. No. (application Ser. No. not yet assigned, Attorney's Docket No. EXPL-011) filed on even date herewith, and titled “Devices, Systems and Methods for Minimally-Invasive Abdominal Surgical Procedures” and co-pending Application Serial No. (Application Serial No. not yet assigned, Attorney's Docket No. EXPL-012) filed on even date herewith, and titled “Minimally-Invasive Methods for Implanting Obesity Treatment Devices”.

### FIELD OF THE INVENTION

The present invention relates to treatment of obesity, more particularly to implantable devices and minimally-invasive methods of implanting a device in the abdominal cavity to treat an obese patient.

### BACKGROUND OF THE INVENTION

Obesity has become a major health concern, both nationally and internationally. The National Center for Health Statistics (NCHS) estimates that over 120 million Americans are overweight, including about 56% of the adult population. Of these, about 52 million are considered obese, as measured by a body mass index (BMI) of 30 or greater. In Europe, an estimated 77 million people are obese, as measured by the same standard. This problem is not limited to western nations, as many developing countries are reported to have obesity rates over 75% of the adult population.

Co-morbidities that are associated with obesity include, but are not limited to type II Diabetes, high blood pressure, sleep apnea, stroke and arthritis, the symptoms of which often tend to be lessened or alleviated upon loss of weight by a person so affected.

In the U.S., options for treatment of obesity are currently quite limited. Current treatment methodologies typically rely upon surgically introducing a “malabsorptive” environment in the gastro-intestinal tract, a restrictive environment or a combination of these. One available treatment method is gastric bypass surgery and another is referred to as gastric banding (one of these techniques is referred to as the LAPBAND™ procedure). These procedures are limited to only those patients with a BMI over 40 (or over 35, with co-morbidities present).

Gastric bypass procedures incur a great deal of morbidity and create a malabsorptive state in the patient by bypassing a large portion of the intestines. Serious side effects, such as liver failure have been associated with this procedure, as well as chronic diarrhea. Another surgical procedure that has a high degree of morbidity associated with it is known as the “Gastric Bypass Roux-en-Y” procedure. This procedure reduces the capacity of the stomach by creating a smaller stomach pouch. The small space holds only about one ounce of fluid. A tiny stomach outlet is also surgically created to slow the speed at which food leaves the stomach. Staples are used to create a small (15 to 20 cc) stomach pouch, with the rest of the stomach being stapled completely shut and divided from the stomach pouch. The small intestine is divided just beyond the duodenum, brought up, and connected to the newly formed stomach pouch. In addition to the considerable morbidity associated with this procedure, other disadvantages include “dumping syndrome”, where stomach contents are literally “dumped” rapidly into the small intestine which may lead to nausea, weakness, sweating, faintness, and diarrhea; hernias resulting from the surgery; gallstones; leakage of the connection between the pouch and the intestine; stretching of the pouch that was formed; nutritional deficiencies; and possible dehiscence of the staples.

The LAPBAND™ is a band that, when placed, encircles the fundus-cardia junction and is inflatable to constrict the same. It does not reduce the volume of the stomach, but rather restricts passage of food into the stomach, the theory being that the patient will feel satiety with a much smaller volume of food than previously. Although the LAPBAND™ procedure is less invasive than a gastric bypass procedure, it also typically achieves less weight loss. Further, it is not a simple procedure and requires a substantial amount of training by a surgeon to become proficient in performing the procedure. Also, a substantial amount of dissecting and suturing is required because the pathway by which the band is introduced is not an existing pathway, and must be established by dissection. Great care is required to avoid blood vessels and nerves that may be in the intended pathway to be created by the dissection. After placing the band around the fundus-cardia junction, the ends of the band must be connected together and then it must be cinched down into place. Additionally, complications such as erosion at the fundus-cardia junction, slippage of the band from its intended location, nausea/vomiting, gastroesophageal reflux, dysphagia and lack of effectiveness in causing weight loss have been reported.

Intragastric balloons have also been placed, in an attempt to fill a portion of the volume in the stomach, with the theory being that it will then require less food than previously, to give the patient a sensation of fullness or satiety. This procedure involves delivery of a balloon (typically, transorally) to the interior of the stomach and inflation of the balloon to take up a portion of the volume inside the stomach. However, intragastric balloons may also lead to complications such as obstruction, vomiting and/or mucosal erosion of the inner lining of the stomach. The balloon can break down over extended exposure to the stomach's acids, and in some cases, after breaking down, the balloon translated through the intestines and caused a bowel obstruction.

Gastrointestinal sleeves have been implanted to line the stomach and/or a portion of the small intestines to reduce the absorptive capabilities of the small intestine and/or to reduce the volume in the stomach, by reducing the available volume to the tubular structure of the graft running therethrough. Although weight loss may be effective while these types of devices are properly functioning, there are complications with anchoring the device within the stomach/GI tract, as the stomach and GI tract function to break down things that enter into them and to move/transport them through. Accordingly, the integrity of the anchoring of the device, as well as the device itself may be compromised over time by the acids and actions of the stomach and GI tract.

A sleeve gastrectomy is an operation in which the left side of the stomach is surgically removed. This results in a much reduced stomach which is substantially tubular and may take on the shape of a banana. This procedure is associated with a high degree of morbidity, as a large portion of the stomach is surgically removed. Additionally, there are risks of complications such as dehiscence of the staple line where the staples are installed to close the surgical incisions where the portion of the stomach was removed. Further, the procedure is not reversible.

In the laparoscopic duodenal switch, the size of the stomach is reduced in similar manner to that performed in a sleeve gastrectomy. Additionally, approximately half of the small intestine is bypassed and the stomach is reconnected to the shortened small intestine. This procedure suffers from the same complications as the sleeve gastrectomy, and even greater morbidity is associated with this procedure due to the additional intestinal bypass that needs to be performed. Still further, complications associated with malabsorption may also present themselves.

An inflatable gastric device is disclosed in U.S. Pat. No. 4,246,893, in which a balloon is inserted anteriorly of the stomach and posteriorly of the left lobe of the liver. The balloon is then inflated to compress the stomach so that it fills with less food that would ordinary be possible. Not only does this device compress the stomach, but it also compresses the liver, as seen in FIG. 5 of the patent, which may cause complications with the liver function. Additionally, the balloon is simply placed into this location, and there is no assurance that it will not migrate and lose its effectiveness in compressing the stomach to the degree intended. Still further, the balloon is of a simple spherical design, and, as such, extends pressure outwardly in all directions, 360 degrees, in all planes. Accordingly, the liver is compressed just as much as the stomach is. Also, the compression forces against the stomach are not ideal, as the spherical balloon conformation does not match the conformation of the expanding stomach. The stomach is not spherical when expanded, or concave with a constant radius of curvature, but expands into a designated space that allows the fundus to expand preferentially more than other parts of the stomach.

Brazzini et al. in WO2005/18417 discloses at least two or more fillable devices used to treat obesity, in which the devices are inserted through the abdominal wall and anchored against the external surface of the stomach wall by an anchoring mechanism that extends through the stomach wall and fixes to the internal surface of the stomach wall.

U.S. Patent Publication No. 2005/0261712 to Balbierz et al. describes capturing a device against the outer surface of the stomach wall to form a restriction that appears to function similarly to the restriction imposed by the LAPBAND™. The anchoring of the devices disclosed relies upon placement of features against the internal wall of the stomach to form an interlock with the device which is placed against the external wall of the stomach.

U.S. Patent Publication Nos. 2005/0267533 and 2006/0212053 to Gertner disclose devices for treatment of obesity that use one or more anchoring mechanisms that are passed through the wall of the stomach to establish an anchor.

U.S. Pat. No. 6,981,978 to Gannoe discloses devices for reducing the internal cavity of the stomach to a much smaller volume, which may be used to carry out a bypass procedure. Stapling is employed to isolate the smaller volume in the stomach, and thus the same potential disadvantages are present as with other stapling procedures described herein.

U.S. Pat. No. 6,186,149 to Pacella et al. describes an occluder device that can be used as a dietary control device (see FIG. 8C). The occluder device is placed against the wall of the stomach and inflated to press inwardly on the stomach wall. A frame is wrapped around the stomach wall and is inflated to press against the stomach wall. However, there is no disclosure of how the frame might be adjusted to maintain a position relative to the stomach wall as the size of the stomach varies.

Gastric reduction techniques have been attempted, such as by inserting instruments trans-orally and reducing the volume of the stomach by stapling portions of it together. However, this technique is prone to failure due to the staples pulling through the tissues that they are meant to bind.

The risk and invasiveness factors of currently available surgeries are often too great for a patient to accept to undergo surgical treatment for his her obesity. Even laparoscopic surgical methods typically require general anesthesia because of the substantial pain otherwise imposed by insufflation. The use of general anesthesia substantially increases the risks of a medical procedure. Morbidly obese patients, in particular are at increased risk of not surviving a procedure involving general anesthesia. Accordingly, there is a need for less invasive, yet effective surgical treatment procedures for morbidly obese patients (patients having a BMI of 35 or greater). Also, since the current surgical procedures are currently indicated only for those patients having a BMI of 40 or greater, or 35 or greater when co-morbidities are present it would be desirable to provide a surgical procedure that would be available for slightly less obese patients, e.g., patients having a BMI of 30 to 35 who are not indicated for the currently available surgical procedures. It would further be desirable to provide a surgical procedure that would be indicated for obese patients having a BMI in the range of 30-35, as well as for more obese patients. It would be desirable to provide less invasive methods that do not require the use of general surgery, to decrease the risk of weight loss reduction surgery, and this would be beneficial for patients in any class or category of obesity. It would further be desirable to reduce pain associated with a procedure for implanting a weight loss device, after completion of the implantation procedure, such as during healing and recovery from the procedure.

### SUMMARY OF THE INVENTION

The present invention provides tools, devices and methods for treating an obese patient.

A method of treating a patient is provided, including: inserting a guide member through an opening in the patient and a delivery tract extending from the opening into the abdominal cavity, and advancing the guide member between the fascia and the abdominal organs of the patient positioning a distal end portion of the guide member at a target location within the abdominal cavity; delivering a conduit over the guide member; removing the guide member, while leaving the conduit in position, directed toward the target location; advancing an implantable device to a distal end of the conduit retracting the conduit to expose the implantable device; securing the implantable device to at least one internal body structure at the target location; at least partially filling a fillable member of the implantable device with fluid; attaching an adjustment member to an inflation tubing in fluid communication with the fillable member and anchoring the adjustment member to an internal body structure; and closing the patient. In at least one embodiment, prior to inserting the guide member, after making an opening through the patient's skin, the delivery tract is established through the opening, subcutaneous fat and fascia, into the patient's abdominal cavity.

In at least one embodiment the guide member comprises a guide, and the method further includes: inserting an endoscope into the guide and the insertion of the guide member comprises inserting the guide and endoscope through the opening in the patient and the delivery tract extending from the opening into the abdominal cavity, and advancing the guide between the fascia and the bowel of the patient, while viewing at least one of the fascia and bowel with the endoscope.

In at least one embodiment, the advancement of the implantable device to a distal end of the conduit comprises inserting a delivery and attachment tool into the conduit wherein the delivery and attachment tool has the implantable device mounted to a distal end portion thereof, the implantable device in a compact configuration, and the endoscope received in the tool and being configured to view locations along the distal end portion of the tool.

In at least one embodiment, the method further includes visually confirming, via the endoscope, that a location between an attachment tab of the implantable device and the target location to which the device is to be attached is clear of bowel, omentum, liver and falciform ligament.

In at least one embodiment, after visually confirming that the location between the attachment tab and target location is clear of bowel, the tool is actuated to deliver at least one anchor into the target location.

In at least one embodiment, the method further includes securing the attachment tab to the target location where the at least one anchor has been delivered.

In at least one embodiment the method further includes detaching the tool from the implantable device and removing the tool and conduit from the patient.

In at least one embodiment, the guide has a flexible distal end portion, and the flexible distal end portion is guided by a patient's anatomy to curve around a curved structure in the patient's anatomy.

In at least one embodiment the conduit has a flexible distal end portion and the conduit is advanced over the curved distal end portion of the guide, the distal end portion of the conduit curving as it is guided by the curved distal end portion of the guide.

In at least one embodiment the actuation of the tool drives a plurality of anchor drivers through at least one internal body structure.

In at least one embodiment the tool drives needles through the at least one internal body structure, the needles carrying anchors.

In at least one embodiment the needles are retracted after being driven through the at least one internal body structure, and the anchors self extract from the needles, are anchored and are not retracted back through the at least one body structure.

In at least one embodiment the tool automatically cinches the attachment tab up against the at least one internal body structure that the implantable device is attached to.

In at least one embodiment the method includes cutting by the tool of excess suture material extending proximally from the attachment tab.

In at least one embodiment, the method further includes dilating an opening in the fascia leading into the abdominal cavity along the delivery tract, prior to delivering the conduit over the guide member.

In at least one embodiment the method includes using a dilator and introducer to perform the dilating, and removing the dilator after the dilating, while maintaining the introducer through the dilated opening, wherein the conduit is delivered through the introducer over the guide.

A method of treating a patient is provided, including: making an incision or puncture though the patient's skin; establishing a delivery tract through an opening formed by the incision or puncture, subcutaneous fat and fascia, into the patient's abdominal cavity; inserting a guide member through the delivery tract and positioning a distal end portion of the guide member at a target location within the abdominal cavity; delivering a first portion of a docking mechanism over the guide member and attaching the first portion of the docking mechanism to at least one internal body structure at the target location; delivering a second portion of the docking mechanism, that is attached to an implantable device in a compact configuration. along the guide member; docking the second portion to the first portion, thereby attaching the implantable device to at least one internal body structure; at least partially filling a fillable member of the implantable device with fluid; removing the guide member; attaching an adjustment member to a conduit in fluid communication with the fillable member and anchoring the adjustment member to an internal body structure; and closing the patient.

In at least one embodiment the implantable device is non-rigidly attached to the at least one internal body structure.

In at least one embodiment the implantable device is tethered to the at least one internal body structure.

These and other features of the invention will become apparent to those persons skilled in the art upon reading the details of the methods, tools and devices as more fully described below.

### BRIEF DESCRIPTION OF THE DRAWINGS

FIG. 1 illustrates the anatomy of the abdominal cavity and its contents, and surrounding features, with one embodiment of an extra-gastric, paragastric device of the present invention shown.

FIG. 2A is an illustration of a diaphragm in an isolated view, illustrating the conformation of the diaphragm as it exists in the body.

FIG. 2B illustrates the diaphragm in position relative to the rib cage.

FIG. 3 is a view of the ribs, not showing the diaphragm, but which shows the costal cartilage portions of the ribcage and the true (bone) ribs.

FIGS. 4A and 4B show views of a device having a main body with a shape and size approximating the shape and size of the full (post-prandial) stomach.

FIG. 5 illustrates (by arrows) potential locations on the stomach wall that can be compressed (or restricted from expanding) lateral to medial; anterior to posterior by one or more devices as described herein.

FIGS. 6A-6P illustrate various parts of a procedure that may be carried out during a single access procedure for percutaneously implanting a fillable paragastric, extra-gastric device according to an embodiment of the present invention.

FIG. 7A illustrates an anchor delivery/attachment tool according to one embodiment thereof.

FIG. 7B shows a distal end portion of the tool of FIG. 7A, illustrating driving of anchor divers out of openings thereof.

FIG. 7C illustrates the tool of FIGS. 7A-7B being used to drive an anchor through costal cartilage.

FIG. 7D illustrates another embodiment of an anchor delivery/attachment tool according to the present invention.

FIG. 7E illustrates another embodiment of an anchor delivery/attachment tool according to the present invention.

FIG. 7F illustrates a visual indicator that identifies to the user of the tool at least the relative amount of deployment of the anchor drivers.

FIG. 7G shows an arrangement where on the visual indicator of the anchor delivery/attachment tool, an automatic retraction indicator is indicated after the actuator fully deploys the drivers.

FIG. 7H shows still another embodiment of an anchor delivery/attachment tool having an actuator provided as a switch or slider.

FIG. 7I illustrates an embodiment of an anchor delivery/attachment tool in which the transverse handle thereof is movable to a stowed configuration.

FIG. 7J illustrates an embodiment of an anchor delivery/attachment tool configured to slidably receive an endoscope therein, or, alternatively, which is configured with an integral endoscope.

FIG. 7K illustrates one embodiment of features that may be included on an anchor delivery/attachment tool, so that the tool can be guided over a guide as described herein.

FIG. 7L illustrates and embodiment of an anchor delivery/attachment tool that is configured to slidably receive an endoscope, and which is provided with a slotted or forked configuration.

FIG. 7M illustrates an anchor delivery/attachment tool in which an implantable device has been inserted into a pod in a compacted configuration and the pod and device have been mounted to the distal end portion of the tool.

FIG. 7N illustrates a device mounted to an anchor delivery/attachment tool according to an embodiment of the present invention.

FIG. 7O illustrates an implantable device according to an embodiment of the present invention.

FIGS. 8A and 8B illustrate one embodiment of a T-bar according to the present invention that is made from a rigid tube (metal or rigid plastic) and has a slot that opens to one open end of the tube.

FIG. 8C illustrates an alternative embodiment of a T-bar according to the present invention.

FIG. 8D illustrates the T-bar of FIG. 8C mounted in a slotted anchor driver.

FIGS. 8E-8F illustrate the automatic or passive extraction of the T-bar of FIGS. 8C-8D from the anchor driver.

FIG. 8G illustrates another embodiment of an anchor mounted in an anchor driver according to the present invention.

FIG. 8H illustrates the anchor of FIG. 8G having been deployed from the driver.

FIGS. 8I-8J illustrate another embodiment of an anchor according to the present invention.

FIGS. 8K-8L illustrate another embodiment of an anchor according to the present invention.

FIG. 9A shows an embodiment of an implantable device, including a fillable member, attachment tab and conduit according to the present invention.

FIG. 9B illustrates compacting a device according to the present invention by rolling.

FIG. 9C illustrates compacting a device according to the present invention by folding.

FIGS. 9D-9E illustrate one embodiment of inserting the compacted device into a pod.

FIG. 9F illustrates a device contained in a pod according to an embodiment of the present invention.

FIG. 9G illustrates a portion of a suture provided with ratchet teeth according to an embodiment of the present invention.

FIG. 9H illustrates ratchet mechanisms provided on the underside of an attachment tab according to an embodiment of the present invention.

FIG. 9I illustrates an embodiment of a speed nut that may be used in performing an attachment/anchoring according to an embodiment of the present invention.

FIG. 9J illustrates speed nuts in the openings of an attachment tab according to an embodiment of the present invention.

FIG. 10A illustrates an alternative mechanism for anchoring an implantable device according to the present invention.

FIG. 10B illustrates a device in a compact configuration being delivered over a guide or guidewire for attachment thereof using the mechanism of FIG. 10A.

FIG. 10C shows another alternative attachment mechanism according to the present invention.

FIG. 10D shows a device being attached by use of the attachment mechanism of FIG. 10C.

FIGS. 11A-11L illustrate various parts of other embodiments of a single access procedure for percutaneously implanting a fillable extra-gastric, paragastric device according to alternative embodiments of the present invention.

FIGS. 12A-12B illustrate an embodiment of a guide according to the present invention.

FIGS. 12C-12E show another embodiment of a guide according to the present invention.

FIGS. 12F-12G show another embodiment of a guide according to the present invention.

FIG. 12H shows another embodiment of a guide according to the present invention.

FIG. 12I shows an embodiment of a guide according to the present invention, showing balloon in an inflated state.

FIG. 12J illustrates a “trap door” feature of an embodiment of a guide according to the present invention.

FIG. 12K illustrates an embodiment of a guide having a balloon, and shows the balloon inflated with an endoscope and a distal end portion of an anchor driving/attachment tool inside the inflated balloon.

FIGS. 12L-12N illustrate additional embodiments of a guide according to the present invention.

FIGS. 12O-12P illustrate a tip and arrangement for connecting the tip to a tube of a guide according to one embodiment of the present invention.

FIGS. 12Q-12T are various views of the tip shown in FIG. 12O.

FIGS. 12U-12W are various views of the band shown in FIG. 12O.

FIGS. 12X-12Y illustrate an alternative arrangement for connecting a tip to a tube of a guide according to the present invention.

13A-13K illustrate various parts of other embodiments of a single access procedure for implanting a fillable extra-gastric, paragastric device in a minimally-invasive manner according to the present invention.

FIG. 14A illustrates a dilator that may be used to perform the dilation of the opening through the fascia and/or abdominal muscle according to procedures of the present invention.

FIG. 14B illustrates a guide extending through the opening in the fascia according to a procedure performed according to one embodiment of the present invention.

FIG. 14C illustrates the dilator of FIG. 14A having been passed over the guide of FIG. 14B and torqued in through the opening in the fascia to dilate it according to the present invention.

FIG. 14D illustrates a large cannula having been passed over the dilator in FIG. 14C and a distal end portion thereof extending through the dilated opening in the fascia according to the present invention.

FIG. 14E shows the large cannula and guide extending through the opening, after removal of the dilator.

FIG. 14F shows the large cannula extending through the opening, after removal of the guide.

FIG. 15A illustrates another embodiment of an anchor delivery/attachment tool that can be used in an alternative embodiment of the procedures described above with regard to FIGS. 13A-13K.

FIG. 15B illustrates the surgeon's/user's ability to visually confirm that no bowel exists between the distal end portion of the tool and the target anchoring sites, prior to contacting the tool thereagainst and actuating the anchor drivers to drive them through the anchoring sites.

FIG. 16A-16D illustrate alternative embodiments of anchors that may be used to anchor a device to an internal body structure according to the present invention.

17A-17O schematically illustrate another example of a single access procedure for percutaneously implanting a fillable extra-gastric, paragastric device according to an embodiment of the present invention.

FIGS. 18A-18P schematically illustrate another example of a single access procedure for percutaneously implanting a fillable extra-gastric, paragastric device according to an embodiment of the present invention.

FIGS. 19A-19T illustrate another example of a single access procedure and variations thereof for percutaneously implanting a fillable paragastric, extra-gastric device according to an embodiment of the present invention.

FIG. 20A illustrates a variation of the method described above with regard to FIGS. 19A-19J, wherein a flexible endoscope is inserted into the guide, rather than a rigid endoscope.

FIG. 20B illustrates use of a conduit in which a least a distal end portion thereof is flexible, and an obturator in which at least a distal end portion thereof is flexible.

FIGS. 21A-21L show an embodiment and variations of a system comprising tools that can be used in carrying out parts of a single access procedure for delivering and implanting a device in a manner as described above, particularly in portions of the procedure of FIGS. 19A-19T, although at least some of the tools shown can be used in one or more of the procedures described prior to the procedure of FIGS. 19A-19T.

### DETAILED DESCRIPTION OF THE INVENTION

Before the present devices, methods and instruments are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a T-bar” includes a plurality of such T-bars and reference to “the suture” includes reference to one or more sutures and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

A “compliant” material refers to a material that is stretchable or fillable. This expansibility allows the material to increase in dimension substantially more than a noncompliant or semi-compliant material, prior to failure. For example, when formed as a balloon structure, a compliant material comprises an expansibility property of being able to increase its radius, beyond its formed radius, under pressure applied into the balloon, by 100 percent or more, without rupturing.

A “noncompliant” material refers to a material that, when formed as a balloon structure, upon filling or inflating can increase its radius beyond its formed radius, under pressure applied into the balloon, only up to about 10 percent or less prior to rupturing.

A “semi-compliant” material refers to a material that, when formed as a balloon structure, upon filling or inflating can increase its radius beyond its formed radius, under pressure applied into the balloon, by an amount between about 10 percent and about 100 percent prior to rupturing.

The “wall” of the stomach refers to all of the layers that make up the stomach wall, including the mucosa, submucosa, muscular layers and serosa. A “layer”, “layer of the stomach wall” or “stomach wall layer” refers to a mucosal layer, submucosal layer, muscular layer or serosal layer.

A “proximal” end of an instrument is the end that is nearer the surgeon when the surgeon is using the instrument for its intended surgical application.

A “distal” end of an instrument is the end that is further from the surgeon when the surgeon is using the instrument for its intended surgical application.

An “internal body structure” when referred to as a structure to which a device is to be anchored, refers to a structure internal to the skin of a patient, and which can be within the abdominal cavity of the patient, or just outside of it such as including the outer surface of a wall that partially defines the abdominal cavity. Structures to which a device can be anchored include, but are not limited to: one or more ribs, particularly the costal cartilage of the ribs, the intercostal muscles, the abdominal surface of the diaphragm, the stomach (but where the anchor does not pass through the wall of the stomach), the anterior abdominal wall, the posterior abdominal wall and the lateral abdominal wall, the esophagus, the angle of His in the stomach, the gastro-intestinal junction, the gastro-esophageal junction, the columnar ligaments of the diaphragm near the gastro-esophageal junction, the superior aspect of the omentum, peritoneum, liver, connective tissues, ligaments, and blood vessels.

An “internal abdominal structure” refers to an internal body structure that is within the abdominal cavity of the patient, including the abdominal wall. For example, attachment to an inner wall surface of the abdominal wall is an attachment to an internal abdominal structure.

A “target volume” of a fillable member 10em is a volume of the fillable member 10em ranging between and including an “initial” volume and a “maximum” volume and which produces efficacious weight loss results when used in a particular patient. Target volume will typically vary from patient to patient.

An “initial” volume of a fillable member 10em is a volume sufficient to fill the fillable to an extent where a minimal amount of or no wrinkles exist in the material of the wall of the fillable member 10em. Typically, the material of the wall of fillable member will not be elastically deformed, or only minimally elastically deformed in the initial volume configuration. When filled to the initial volume, the fillable member typically has about half the volume (e.g., about fifty to sixty percent of the target volume) that is has in the target volume configuration, but the walls of the fillable member are under sufficient pressure so that there are no wrinkles in the walls. By configuring fillable member 10em to be reduced from a target volume configuration to the initial configuration, this gives the surgeon considerable leeway to adjust the volume of the fillable member 10em downwardly, while still maintaining the fillable member 10em in a configuration which substantially prevents bending, creasing and/or erosion of the materials forming the wall of the fillable member 10em.

A “Max” or “maximum” volume of a fillable member 10em is greater than the “target volume”, typically at least about fifty percent greater than the target volume. This allows the surgeon to safely fill the fillable member to a volume configuration that is at least about fifty percent larger than the target volume, if needed.

The preferred embodiments of the present invention facilitate minimally-invasive implantation of an implant to treat obesity. A patient is treated by the any of the present methods, devices and systems, to implant at least one implantable device by a single access procedure to facilitate weight loss in the patient. In preferred embodiments, a minimally-invasive procedure does not require putting the patient under general anesthesia and insufflation of the abdominal cavity is not required. Preferably, only a single small opening is required for delivery of the device into the abdominal cavity. The small opening will generally be less than about 2.5″ in diameter, typically less than about 2.2″ in diameter, or less than about 2.0″ in diameter, or less than about 1.5″ in diameter, or less than about 1.25″ in diameter or about 1.0″ in diameter or less. Alternatively, more than one opening may be used for use in viewing through and/or inserting additional instruments.

The preferred embodiments of the present invention prevent the possible issue of erosion caused by a fillable member, by not requiring anchoring to the stomach, and further, by not requiring a substantial compression force to be applied when the stomach is not full of food. By allowing the stomach to move freely in the constrained space provided by the fillable member, the stomach's possible expansion size will be decreased, but there will be less opportunity for the formation of pressure necrosis since no one region will be subjected to concentrated forces. With the device in place, there is substantially no distensibility of the stomach as normally exists with an unconstrained stomach. With distensibility restricted and gastric volume reduced, as the patient ingests food, the intra-gastric pressure will rise to a level sufficient to produce satiety without distension or volume expansion of one or more regions of the stomach. The device occupies so much volume in the abdominal cavity that the stomach does not substantially depart from the shape set by the device even when filled with food. Another physiological benefit of the device is that the stomach's ability to relax in response to ingestion of food is reduced or eliminated, thereby producing earlier satiety. One additional physiological benefit of the fillable member may further be to substantially reduce the actual volume of the stomach itself, remodeling the organ as the muscle contracts into its new shape over the period of weeks or months (just as the heart remodels when constrained from over-expansion). Remodeling the stomach allows the fillable member to be implanted temporarily.

The preferred embodiments also are positioned in a paragastric, extra-gastric location to substantially fill the space normally occupied by the fundus and body of the stomach, thus moving the stomach medially and wedging the stomach between the fillable member and the medial and anterior aspects of the liver, and the spine posteriorly. This position also ensures that the fillable member is almost entirely maintained underneath the diaphragmatic umbrella beneath the ribs on the left side, thus concealing the fillable member, and preventing it from producing an unsatisfactory cosmetic result.

Further, the preferred embodiments can have elements for anchoring on one or more locations along the costal cartilage features of the ribs and/or abdominal cavity wall to prevent migration. Further, the preferred embodiments are provided with an outer surface that is very atraumatic. Embodiments described may include at least one fillable member, preferably an inflatable member, made of a material or material composite that is impermeable to fluid, which may be substantially impermeable to gas.

Other embodiments include those having at least two fillable members, with one fillable member being inflated with a gas and another fillable member being inflated with a liquid, or those having a fillable member and a buoyancy member that may or may not be fillable, and which adds buoyancy to the device. These embodiments are less desirable however, as they may require a larger opening for implantation thereof.

### Abdominal Cavity Anatomy

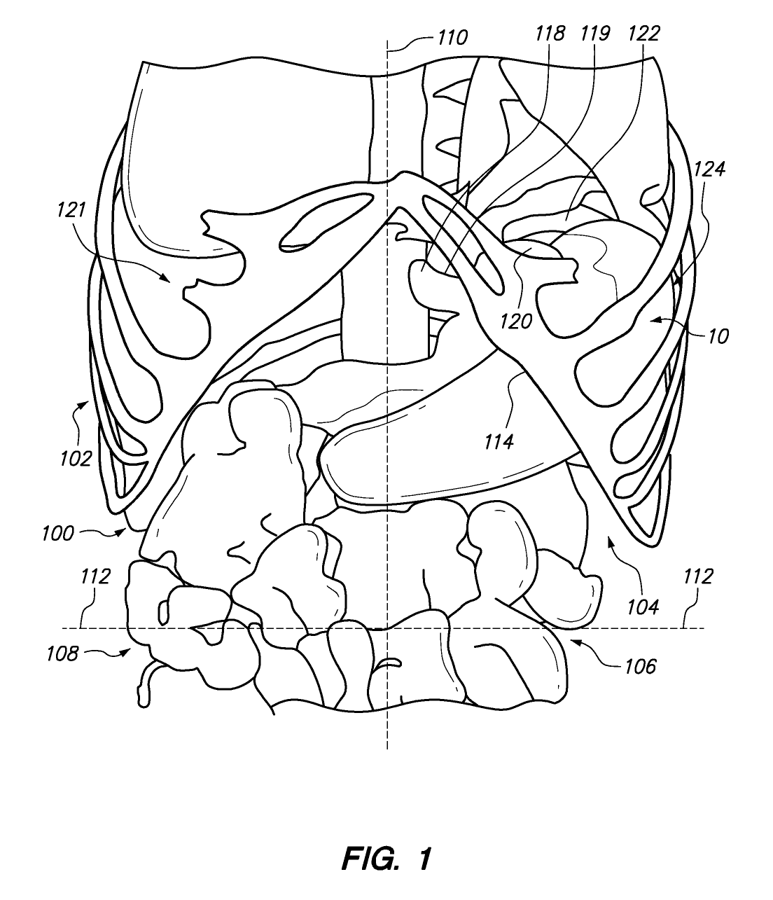

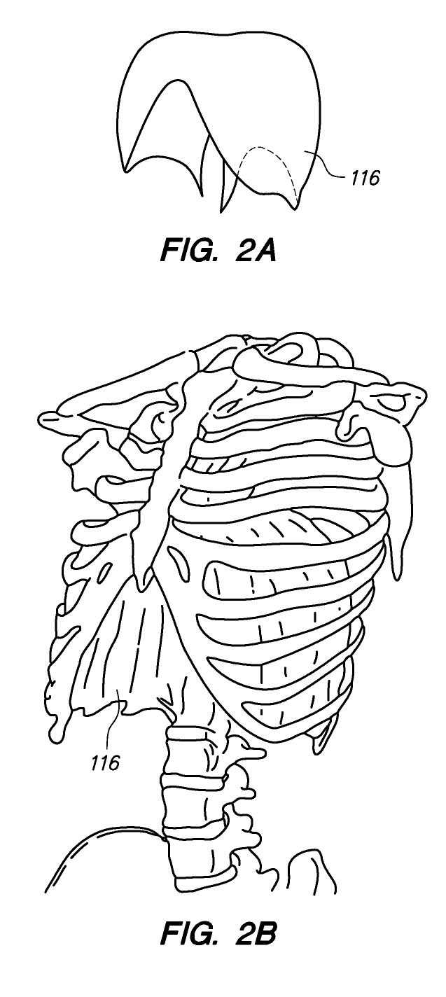

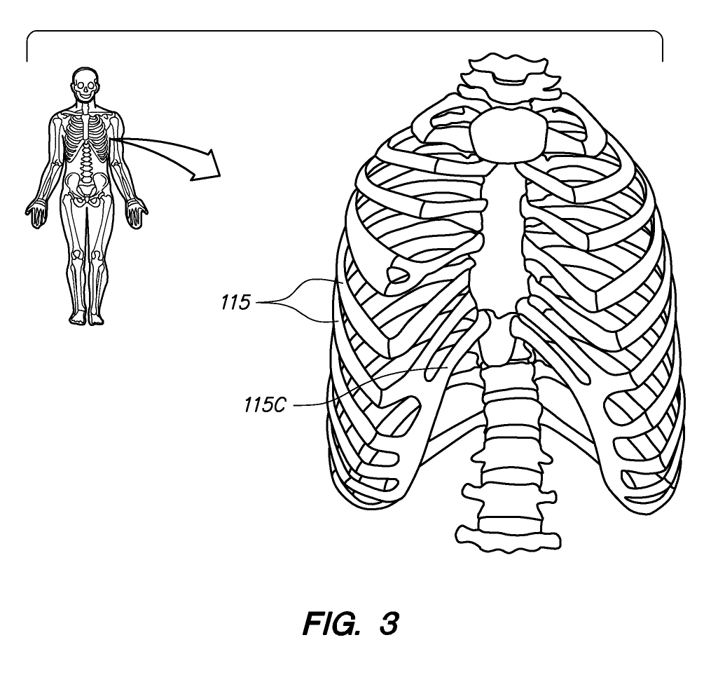

FIG. 1 illustrates the anatomy of the abdominal cavity and its contents, and surrounding features. The abdominal cavity 100 is shown divided among four quadrants, the upper right quadrant 102, upper left quadrant 104, lower left quadrant 106 and lower right quadrant 108, as divided by the median axis 110 and transverse axis 112. The lower edge of the ribcage is illustrated at 114 and the diaphragm 116 is not shown in FIG. 1, but is illustrated in FIGS. 2A-2B. As can be seen in FIGS. 2A and 2B, the diaphragm 116 is shaped like a parachute and sits within the ribs. FIG. 3 is a view of the ribs, not showing the diaphragm 116, but which shows the costal cartilage portions 115c of the ribcage and the true (bone) ribs 115. The esophagus 118 passes through the diaphragm 116 and joins with the stomach 120. The left lobe 122 of the liver 121 lies anteriorly of the esophagus 118 and the fundus-cardia junction 119. In one aspect of the invention, a fillable device 10 is implanted in an extra-gastric location (i.e., outside of the stomach) generally indicated at 124, and then expanded to occupy at least a space that the fundus of the stomach would ordinarily expand into when the stomach is filled with food. The expanded device prevents this expansion by the fundus, thereby limiting the volume of the cavity in the stomach to a much smaller volume than if the fundus had been allowed to expand into the space. Alternatively, the device is expanded to apply pressure to at least the fundus of the stomach in a downward direction (e.g., in a direction toward the transverse axis 112 shown, with some transverse movement toward the median axis 110 shown), and optionally, additionally to the main body of the stomach, to reduce the volume inside the stomach to effect satiety in the patient with relatively less food ingested, relative to what the patient would require for satiety without the implant in place.

### Devices

Devices described herein can be implanted percutaneously, with a relatively quick and simple procedure may not require general anesthesia (although general anesthesia can be used at the surgeon's discretion) and wherein only a single, small opening in a patient is required to deliver the device, which typically has a single fillable member that is self anchoring or can be easily anchored to maintain the simplicity and minimal invasiveness of the procedure.

In preferred embodiments, a device is provided with a single fillable member so as to minimize the cross-sectional area of a device when compacted to be inserted through a single access percutaneous opening. Alternatively, a device can contain one or more fillable members, although this will typically require a larger opening in the patient through which to insert the device into the abdominal cavity. Any of the devices described herein can, be implanted using open surgical procedures, laparoscopic procedures and/or single port access procedures.

Devices described herein can be implanted permanently, but are also configured for reversibility, to facilitate relatively simple removal procedures, should it be desired to remove a device. Alternatively, devices according to the present invention can be implanted temporarily, such as over a period of months, and then removed or disabled when further treatment is no longer required, or to allow an alternative treatment to be applied.

### Device Body Configurations

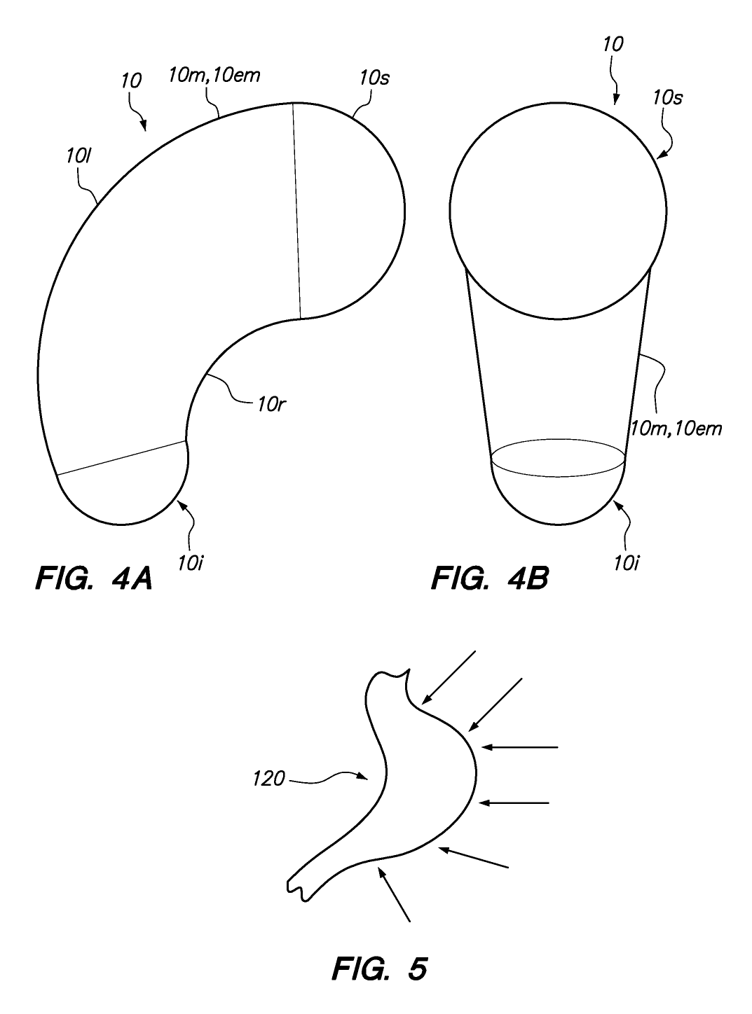

FIGS. 4A and 4B show views of a device 10 having a main body 10m,10em with a shape and size approximating the shape and size of the full (post-prandial) stomach 120. Although main body 10m need not be fillable/collapsible to perform restriction of stomach expansion, main body 10m is typically formed from one or more fillable members 10em, preferably only one fillable member 10em, as noted above, for better performance of intended functions and to allow less invasive procedures for implanting the same.

Main body 10m,10em includes curved left and right sides 10l and 10r, respectively (FIG. 4A shows the posterior surface of main body 10m,10em), wherein the left side 101 is convex and the fight side 10r is concave such that the main body 10m, 10em takes on the shape of a portion of the full stomach that expands from the shape of a substantially empty stomach. The superior portion 10s is substantially larger and more bulbous than the inferior portion 10i, since the fundus portion of the stomach 120 expands much more than the body of the stomach and antrum upon receiving food. Thus, as seen in the right side view of FIG. 4B, the superior portion 10s is very bulbous and almost spherical, with a larger cross section than the inferior portion 10i, while the inferior portion is more nearly hemispherical, with the center portion of the main body tapering from the superior portion 10s to the inferior portion 10i. Configured as such, the main body 10m,10em, when implanted properly, will occupy the space that naturally exists from the stomach 120 to expand into when expanding from a pre-prandial configuration to a post-prandial configuration. By severely limiting this expansion capability, the patient is thereby able to consume only a significantly smaller volume of food than possible if the implant were not present.

Device 10 sizes may vary depending on the size of the skeletal system of the patient into which device 10 is to be implanted, particularly the size of the rib cage. Further variations may be made to tweak or adjust the amount of restriction along any desired location of the stomach that interfaces with device 10. One typical variation is in the length and/or size (diameter or expandability capacitance) of the inferior portion 10i. In some embodiments, the inferior portion 10i of the fillable member 10em may be made longer than shown in FIGS. 4A-4B to extend further inferiorly and medially than the inferior portion of the fillable member shown in FIGS. 4A-4B.

At least a portion of main body member 10m may be fillable. The entire main body 10m may be made of a fillable member 10em. When in an expanded configuration, fillable member 10em can optionally only abut or lie adjacent to the pre-prandial stomach wall, without imparting any significant concentrated deformation forces thereto. However, when the patient eats and the stomach begins to fill, fillable member 10em in this case prevents the stomach 120 from expanding into the volume occupied by fillable member 10em. In such a case, the stomach 120 becomes “deformed” as it attempts to expand and can only expand in a limited fashion, if at all, around a portion of the perimeter of fillable member 10em. Thus, upon expanding the device 10, the device 10 expands in the space(s) normally occupied by the stomach 120 as the stomach 120 expands when receiving food. Thus device 10 exerts pressure on, or at least prevents expansion of the fundus and optionally, the antrum. In embodiments where the fillable device 10 is not attached to the stomach, the stomach is free to perform its normal function of mixing food in the stomach for digesting and pushing food out of the stomach. During all of this movement the stomach may slip behind, beside or on top of the fillable device, but the internal volume of the stomach will be held to its smaller volume as the fillable device 10em is occupying the space into which the stomach would normally expand. Further details of methods for treatment of obesity, including procedures for implanting devices described herein are described below.

As noted above, a fillable device 10 can be implanted adjacent a surface of the stomach wall, either in contact therewith or at a predetermined distance therefrom, to prevent expansion of the stomach 120 into a volume occupied by the fillable device 10. Alternatively, some embodiments of the devices described herein can be configured and placed to exert an external compression on one or more locations of the stomach to deform the stomach wall, thereby decreasing the internal volume of the cavity within the stomach that accepts food and liquid intake. FIG. 5 illustrates (by arrows) potential locations on the stomach 120 wall that can be compressed (or restricted from expanding) by one or more devices 10 as described herein.

In at least one embodiment fillable member 10em shown in FIGS. 4A-4B is composed of an inflatable member 10em. Inflatable members described herein can be inflated with gas or liquid or both. Examples of gases or liquids that can be used to inflate inflatable members/devices 10 include, but are not limited to: carbon dioxide, helium, isotonic dextrose solution, saline solution (e.g., isotonic saline solution), air.

At least a portion of the fillable member 10em shown in FIGS. 4A-4B may be inflated with one or more gases, to provide a relatively lighter, less dense implanted device 10, relative to a fillable member completely filled with liquid. The entire fillable member 10em may be inflated with one or more gases. Alternatively, the entire fillable member 10em may be inflated with one or more liquids. Further alternatively, devices 10 can be at least partially inflated with a porous gel that is porous or microporous to encapsulate air or other gas bubbles, thereby reducing the weight of the gel while still permitting it to apply volumetric pressure to expand an inflatable member. Such gels may be settable, such as ultra-violet (uv) curable or otherwise chemically curable, or, alternatively, can remain in the gel state, so that they can be readily removed or added to, to increase or decrease the amount of inflation expansion of the fillable member. Gels can be made from a flowable viscoelastic substance made of a polymer mixture, such as silicone oil, boric acid, hyaluronic acid, polyacrylic acid or combinations thereof, for example. The gel, as delivered into the fillable member 10em (e.g., such as by injection or the like) can be aerated or infused with carbon dioxide or an inert gas to create a deformable or non-deformable cellular structure that encapsulates the gas in cells, and thus has relatively low mass but still has significant resistance to compression or deformation.

Other embodiments of devices that may be configured for delivery according to at least one of the methods described herein are disclosed in co-pending, commonly owned application Ser. Nos. 11/407,701; 11/881,144; 11/716,985; 1/716,986 and 11/974,444. Each of application Ser. Nos. 11/407,701; 11/881,144; 11/716,985; 11/716,986 and 11/974,444 is hereby incorporated herein, in its entirety, by reference thereto.

### Methods and Instruments/Tools

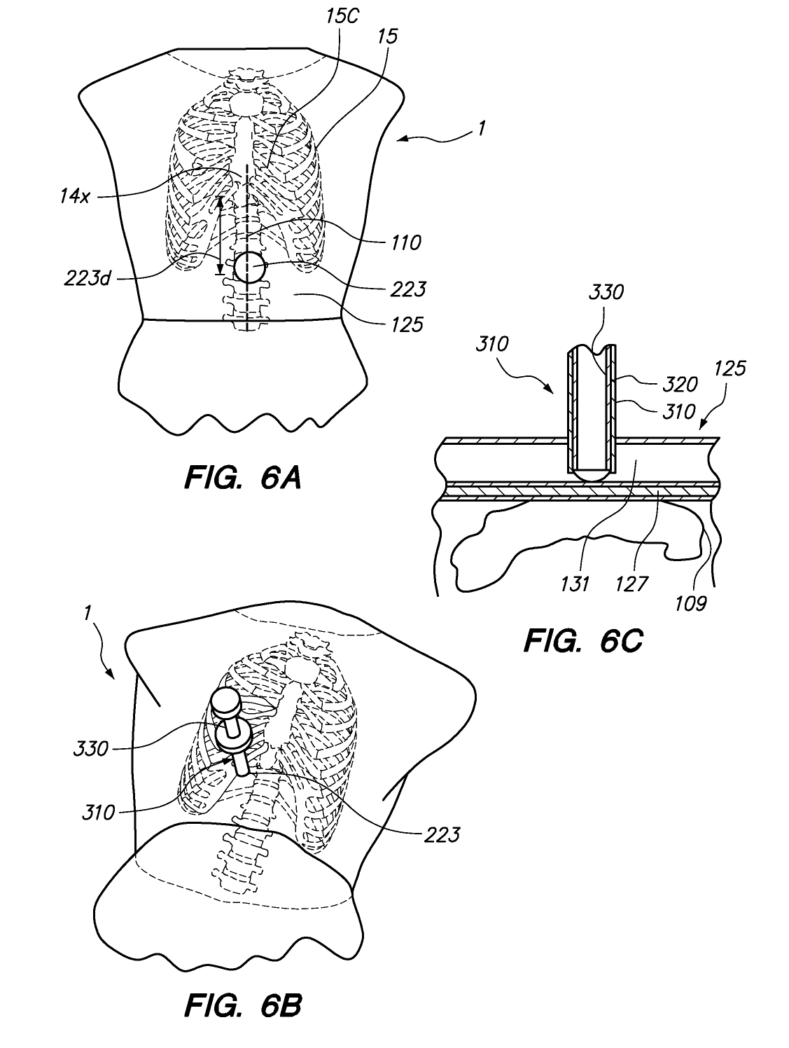

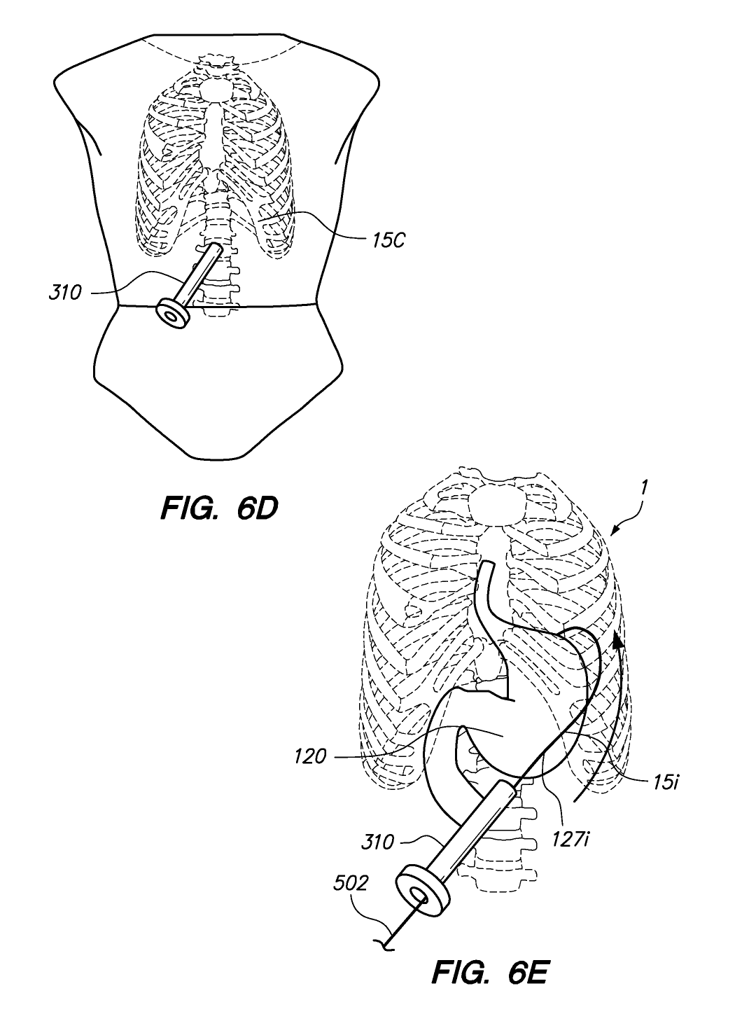

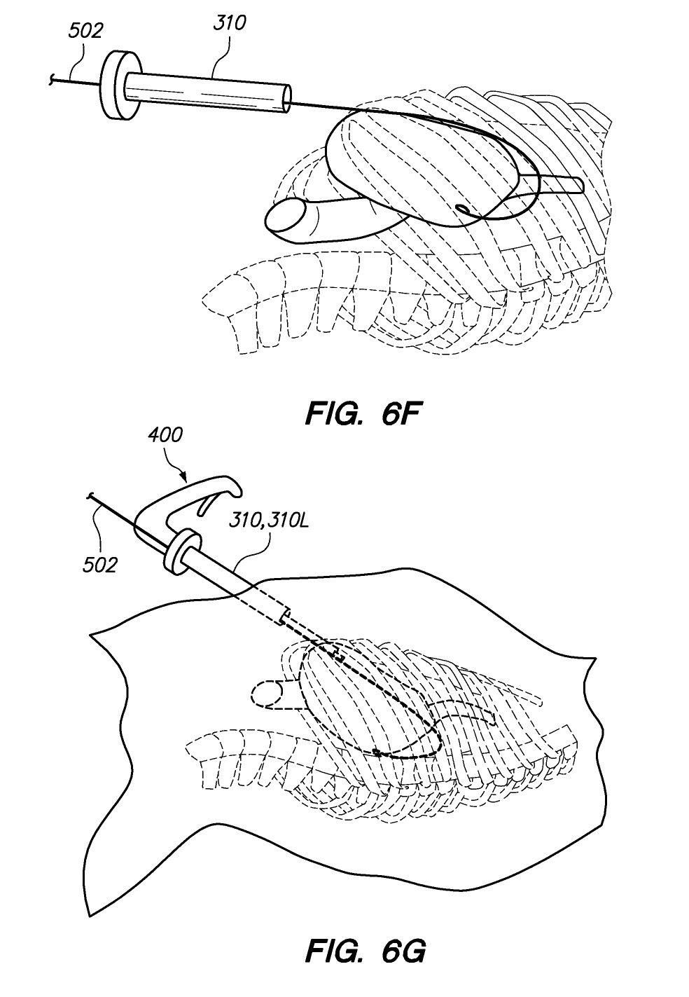

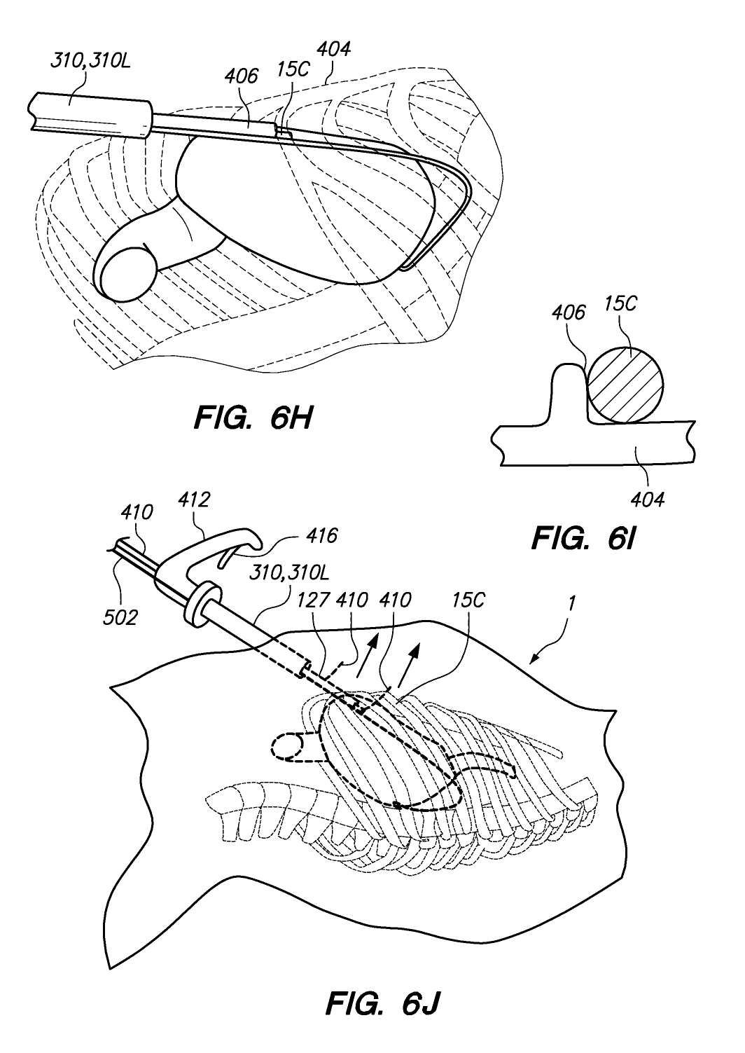

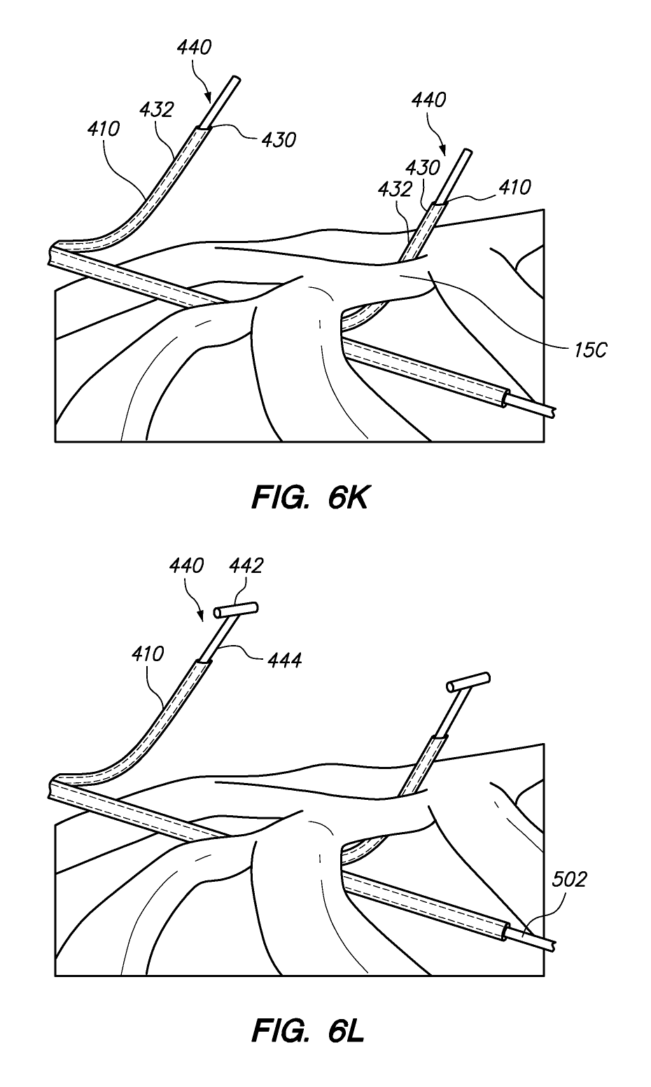

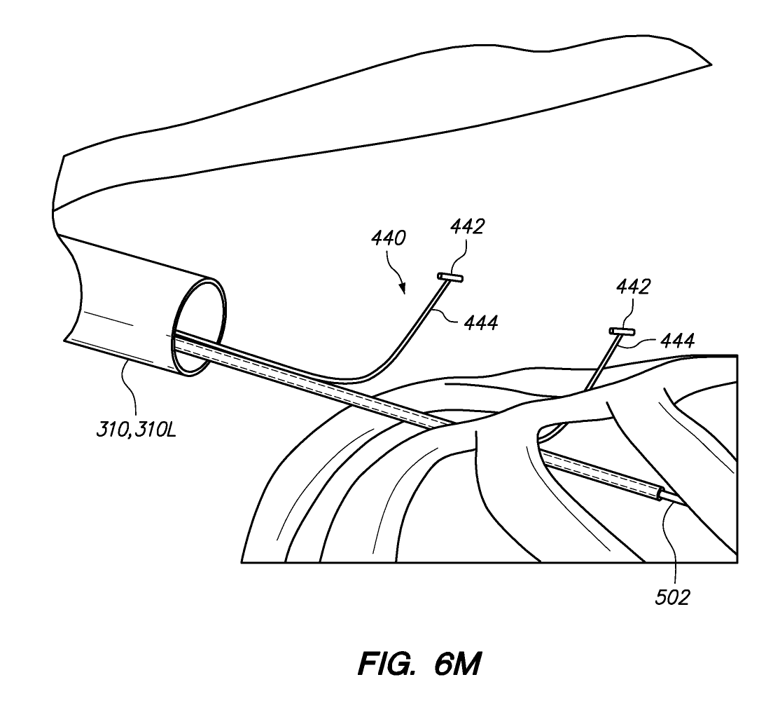

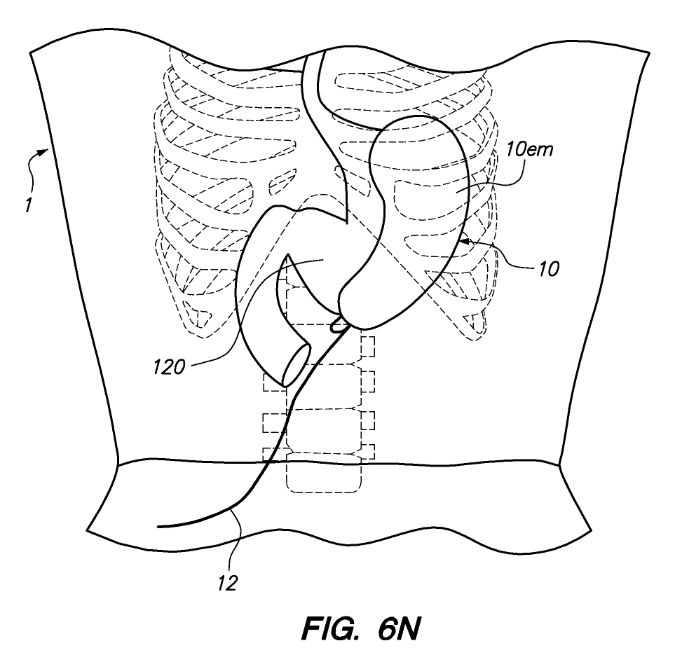

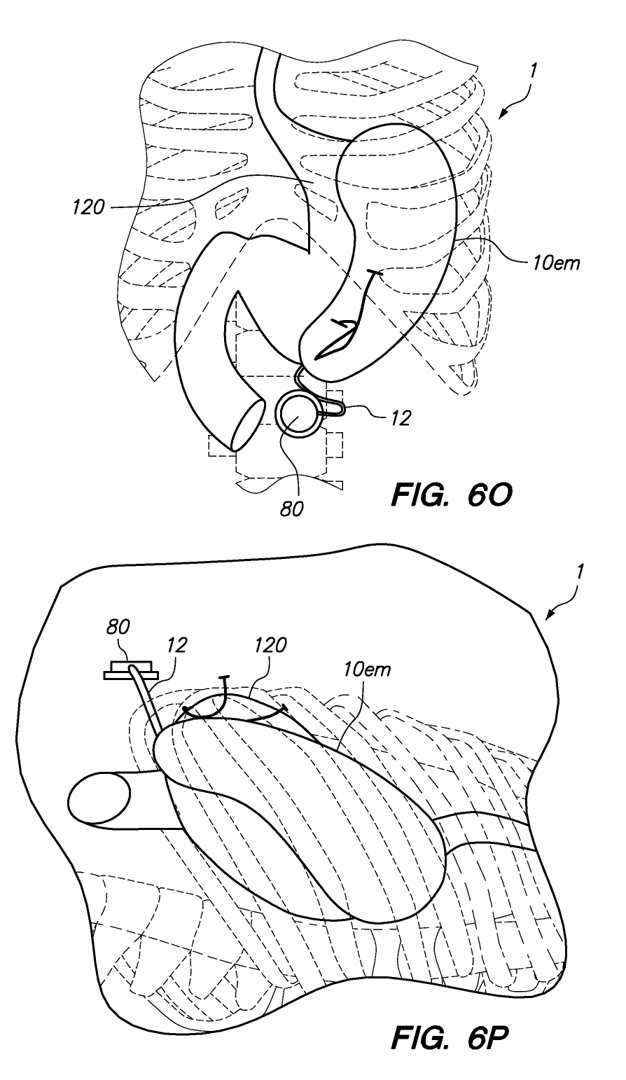

FIGS. 6A-6P illustrate various parts of other embodiments of a procedure that may be carried out during a single incision access procedure for percutaneously implanting a fillable extra-gastric device 10 according to an embodiment of the present invention. Prior to making an incision, the local area (the area of the skin in and surrounding the location where the incision is to be made) may be prepared by disinfecting with alcohol and/or betadine. Additionally, the patient may be given a mild sedative, or may be on conscious sedation, but no general anesthesia may be required to the extent that the patient becomes unconscious. However, the surgeon may use general anesthesia at the surgeon's discretion, as there is nothing about the methods described herein that cannot be carried out under general anesthesia. However, the present methods are designed to preferably be carried out without the use of general anesthesia. Next a powerful local anesthetic such as marcaine (bupivicaine) or other powerful anesthetic, optionally mixed with an epinephrine or other vasoconstrictor to reduce any bleeding that might result from mild trauma, is injected into the local area through the skin 125 of the patient 1 down to the muscular layer and to infiltrate the fat layer and entire local area. Injection may be performed using a syringe or other injection tool. After allowing time for the injected anesthesia to take effect a small incision 223 (e.g., no greater than about seven cm or no greater than about five cm, typically less than 2 cm, even more typically about 1 cm or less]) is made in the skin 125 of the patient 1, with a scalpel or other surgical cutting tool, in the local area over the surgical target area where device 10 is to be implanted. In the example shown, the incision 223 is made along the median axis 110, at a distance 223d about 6 cm to about 10 cm inferior of the xiphoid process 15x. However, the location of the incision may be varied. For example, the incision 223 may be made to the left of the median axis 110 as shown in FIG. 6A (right side of patient 1). Likewise, the distance 223d from the lower rib line 114/xiphoid process 14x may also vary.

A delivery tract is then formed by inserting a trocar cannula system 310 into incision 223 as illustrated in FIG. 6B. Because the procedure does not include the use of insufflation, there is no gas pocket created in the abdominal cavity between the abdominal wall/fascia and the bowels and other internal organs in the abdominal cavity like there is during a laparoscopic procedure. Accordingly, care must be taken when forming the delivery tract to avoid trauma to internal organs, particularly the bowel 109. For this reason, trocar cannula system 310 is provided with a clear-lipped trocar 320 that is inserted through cannula 310 to form the opening through the abdominal wall 127, thereby establishing the delivery tract. Additionally, trocar 320 includes a central annulus that opens to the proximal end of the trocar 320 and is dimensioned and configured to receive the distal tip and shaft of an endoscope 330 therein. The clear distal tip of trocar 320 is plastic and forms a semi-sharp tip, such as by including molded plastic edges or blades that allow the tip to be rotated to drill through tissue as pressure is also applied to the tissue via the tip. This, together with the visualization through the tip provided by the endoscope 330 received therein allows a controlled, deliberate manner of drilling that can be visualized via the endoscope. Accordingly, the drilling shows a generally yellow visualization during drilling through the fat layer 131. When the tip of the trocar 320 abuts the fascia overlying the abdominal muscle 127, the visual field changes to a white color, thereby alerting the surgeon to the fact that the drilling is now proceeding through the fascia and abdominal muscle. Once the opening reaches the abdominal cavity (i.e., external fascia, abdominal muscle and internal fascia have been drilled through, or, in the case of midline drilling, it may be mainly fascia and little to no abdominal muscle), the white field spreads out as the trocar passes through the fascia and the visual field typically returns to yellow indicating visualization of the fat within the abdominal cavity.

Once the delivery tract has been opened into the abdominal cavity, as visually confirmed via the endoscope 330, the endoscope 330 and trocar 320 are removed from the cannula 310, while leaving the cannula 310 in place through the delivery tract and extending into the abdominal cavity. The cannula 310 is then tilted, as illustrated in FIG. 6D, so as to point toward the location where the device is to be implanted. Cannula 310 is also angled at a shallow angle (e.g., about 60 to about 90 degrees or more from the initial perpendicular orientation), so that tools or devices inserted through the cannula 310 are directed under the costal cartilage 15c locations and up and around a pathway generally following the diaphragm 116.

Once the orientation of the cannula 310 is established as described above, a guidewire 502 is inserted through cannula 310 which directs the trajectory of the distal end portion of the guidewire up and underneath the costal cartilages 15c to ride around the curvature of the diaphragm 116. Note that in FIG. 6E, the resulting curvature of the distal end portion of guidewire 502 is shown as it rides along the inferior surface of the diaphragm, but, for simplicity of illustration and explanation, the diaphragm 116 is not shown in FIG. 6E. It can be helpful to use 0.5 liters or less of carbon dioxide insufflation to create a small passage for the guidewire (or any of the other guide members described below) to be advanced in. This small amount of insufflation allows the procedure to still be done under local anesthesia, but helps confirm to the physician that he/she has the guidewire or other guide device along the surface of the fascia and above the bowel.

The trajectory of the distal end portion of the guidewire 502 can be visually followed using fluoroscopy. Guidewire 502 will typically have one or more radiopaque markings that will facilitate visual tracking by fluoroscopy, and or, the metallic portions of the guidewire itself can generally be visualized under fluoroscopy.

FIGS. 6E and 6F illustrate two different viewing angles that are useful in tracking the guidewire 502 as it moves around the curvature of the diaphragm, FIG. 6E being viewed from a location anterior of the patient 1 and FIG. 6F being viewed from a location along the left side of the patient 1. This viewing by fluoroscopy can confirm to the surgeon that the guidewire has correctly traveled near and around the stomach 120 and along the curvature of the diaphragm, and the viewed placement of the guidewire 502 shows where the device 10 will be placed, so the surgeon can determine by viewing the placement of guidewire 502 whether the device will be placed as desired. As placed, guidewire 502 acts as a kind of track or rail over which the device 10 can be delivered and properly placed. Further, by viewing the intersection 15i of the guidewire 502 with the lowermost costal cartilage 15c, this provides a good indication of where the device 10 can be anchored to the costal cartilage. An additional anchor location 127i (e.g., through the fascia and abdominal wall) can be located by following the trajectory of the guidewire 502 from 15i to a location slightly inferior of 15i, by the distance between anchor points on an attachment feature of the device 10, which will be described in more detail below.

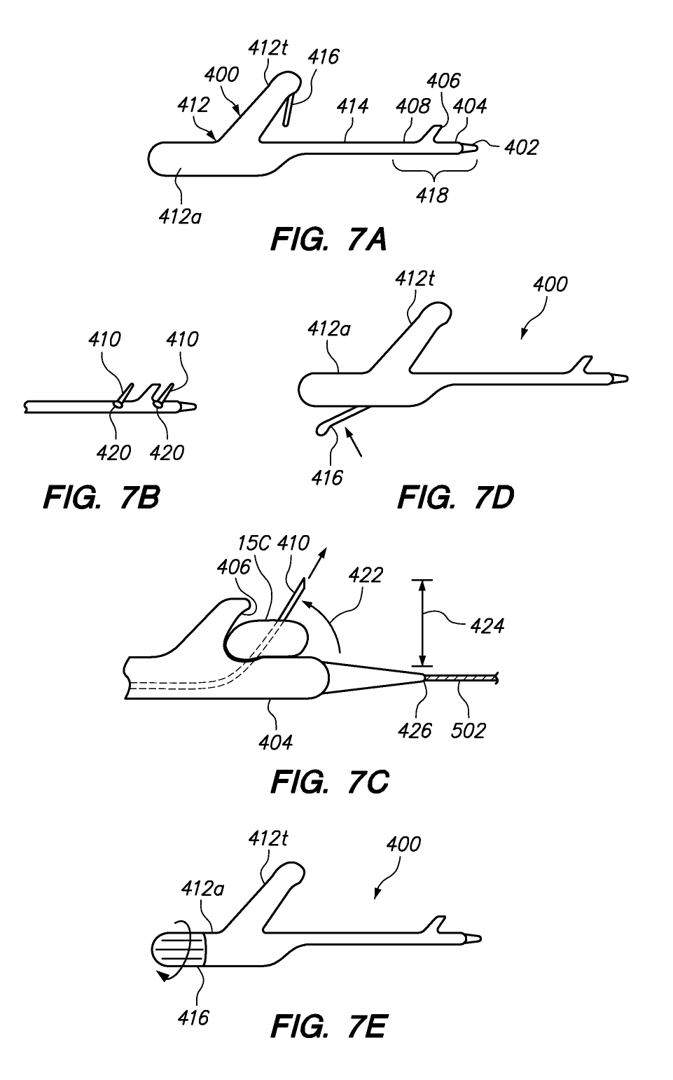

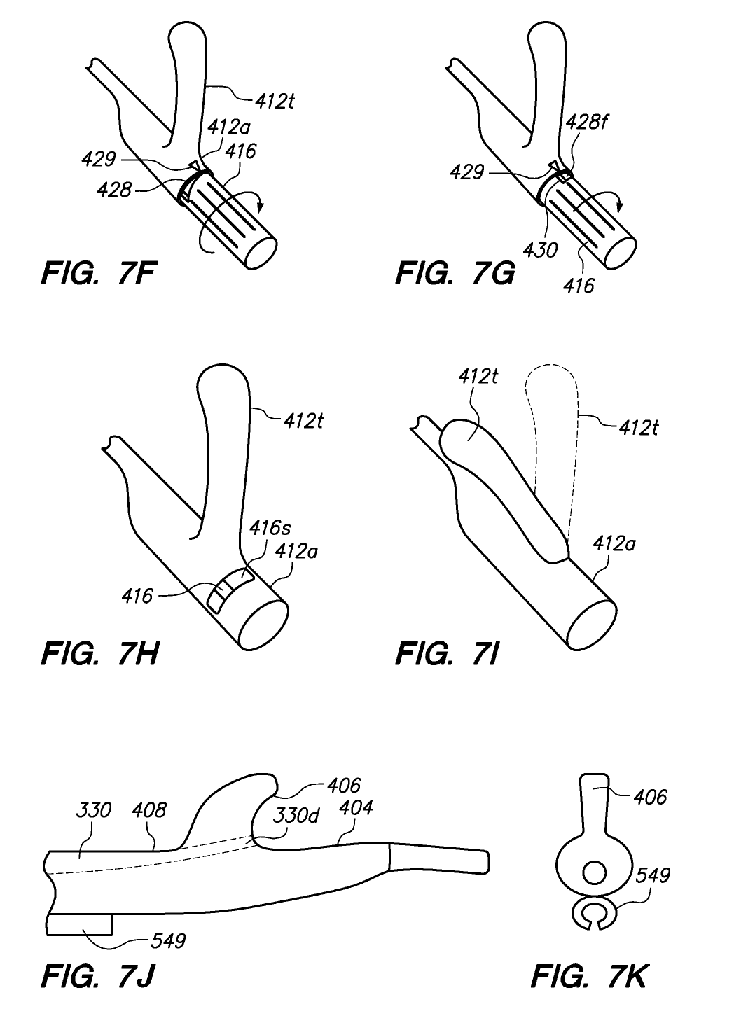

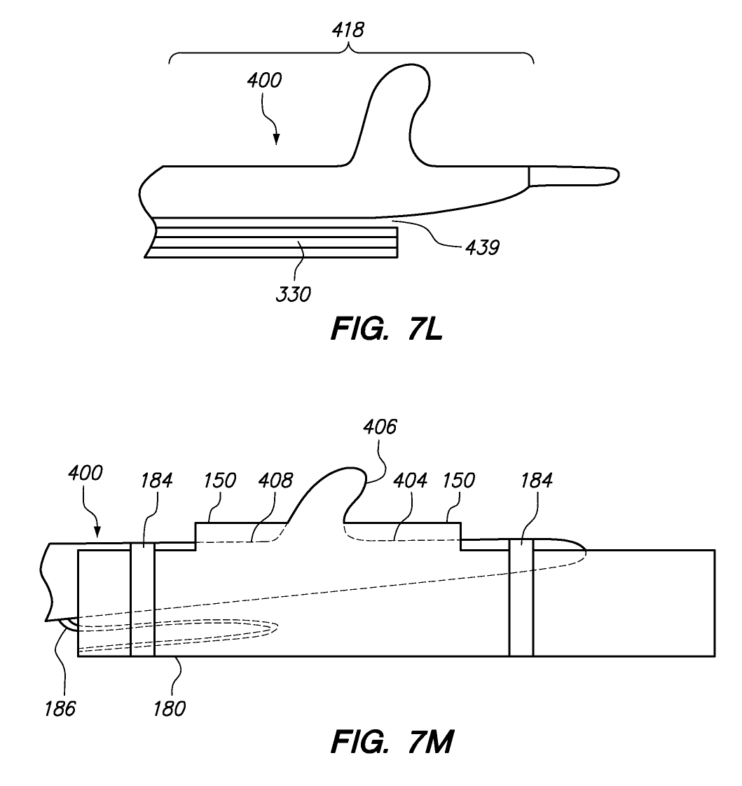

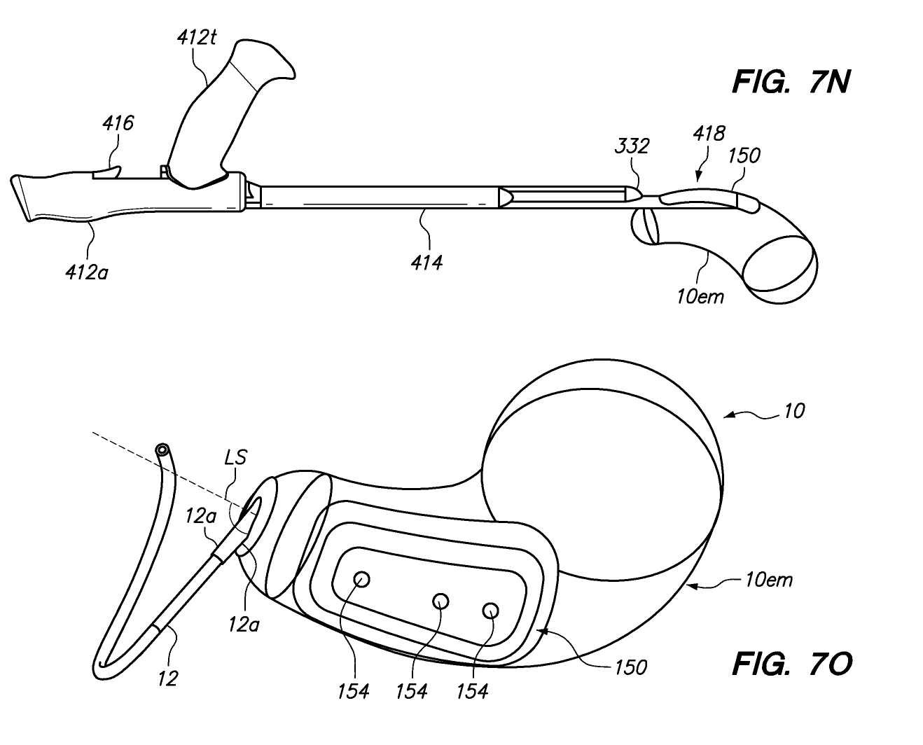

Once the guidewire 502 is oriented as desired, an anchor delivery tool 400 is inserted through cannula 310 as illustrated in FIG. 6G. Tool 400 includes a handle 412, a rigid, hollow shaft 414, a distal end portion 418, and an actuator 416 operable to drive anchor drivers 410 out of openings 420 (FIGS. 7A-7B) and through tissues to be anchored to. The distal end portion 418 of tool 400 includes a flexible distal tip 402 (e.g., see FIG. 7A) to prevent traumatic contact with or damage to the tissues or organs in the abdominal cavity as tool 400 is being manipulated operated. A rigid ledge portion 404 of the distal end portion is located just proximal of the flexible distal tip 402. Rigid ledge portion 404 is configured to be passed under the costal cartilage 15c and slide thereagainst until stop member 406 abuts against the costal cartilage 15c and prevents tool 400 from being further advanced. FIG. 6H illustrates stop member 406 abutting against costal cartilage 15c with rigid ledge portion 404 in underlying contact with the costal cartilage 15c. FIG. 6I is an enlarged illustration of the stop member 406 in contact with the costal cartilage 15c (illustrated in cross-section) with rigid ledge 404 in underlying contact with the costal cartilage 15c.

Tool 400 further includes a proximal drive portion 408 just proximal of stop member 406. Both proximal dive portion 408 and rigid ledge portion 404 are provided with anchor drivers 410 that are driven out of openings 420 when actuator 416 is actuated by an operator. Stop member 406 may extend up from the contact surface of rigid ledge portion 404 by a height of about 0.75 cm to about 3 cm, and the distance between openings 420 may be in the range of about 3 cm to about 7 cm, for example. The opening 420 in the rigid ledge portion 404 is located so that when the distal end portion 418 is abutted against the costal cartilage 15c (typically the most inferior costal cartilage 15c) as described above and shown in FIG. 6I, anchor driver 410, upon actuation by actuator 416, is driven through the costal cartilage 15c. The opening 420 in the proximal drive portion 408 is spaced from the opening 420 in the rigid ledge portion 404 by a distance that corresponds to a distance between two anchor points on the device 10 (or attachment tab 150 of device 10) as described in further detail below.

FIG. 7C illustrates the anchor driver 410 having been driven through the costal cartilage 15c. In order to facilitate the delivery of the drivers 410 out of the tool 400, the drivers 410 need not be driven out at a perpendicular orientation to the tool 400, as an angle 422 of about 45 degrees to about 60 degrees is generally sufficient. Also, the perpendicular distance or height 424 that the distal tips of the anchor drivers 410 extend from the surface of the tool 400 is typically in the range of about 30 mm to about 40 mm. Of course this distance can be varied as needed, depending upon the measurements of the particular patient being treated, so as to ensure that anchors are delivered to a required distance above the surface of the tool 400. It should be further noted here that tool 400, when inserted through cannula 310, may be inserted adjacent to guidewire 502 as illustrated in FIG. 6G above, since tool 400 is substantially rigid and therefore guided to the target location by cannula 310, but alternatively, and preferably is provided with a lumen 426 that allows tool to be passed over guidewire 502 (see FIG. 7C) when tool 400 is inserted into cannula 310.

FIG. 6J schematically illustrates the driving of anchor drivers 410 through the costal cartilage 15c and fascia/abdominal muscle 127, respectively. Handle 412 is used to maintain leverage on tool 400 to keep the rigid ledge portion 404 and stop 406 in contact with the costal cartilage 15c while actuating the actuator 416. Upon such actuation, anchor drivers 410 are driven out into the respective tissues as described above and as illustrated in FIG. 6J.

In at least one embodiment, the anchor divers 410 comprise hollow needles each having a lumen 430 passing therethrough along the direction of the longitudinal axis thereof, as illustrated in the enlarged, partial schematic view of FIG. 6K. Anchors 440 are inserted through the lumens 430 of anchor drivers 410. After piercing though the respective tissues by driving the anchor drivers 410 as described and illustrated in FIG. 6J, anchors 440 are deployed out of the distal openings of anchor drivers 410 as illustrated in FIG. 6K, such as by pushing them out with a pusher rod 432. Alternatively, in other embodiments, anchors 440 may be passively deployed as they catch against tissue to maintain their relative positions while the anchor drivers 410 are retracted. It is noted that handle 412, shaft 414, proximal driver portion 408, stop 406, rigid ledge portion 404 and flexible distal tip 402 of tool 400 are not shown in FIGS. 6K-6N, for clarity and simplicity of showing the functions of the anchor drivers 410 and anchors 440, but that the tool 400 would still be in place as shown in FIGS. 6H and 6J during the procedures of FIGS. 6K-6L. Anchors 440 may be simultaneously deployed, or deployed one after the other. Similarly, the driving of the anchor drivers through the tissues as described with reference to FIG. 6J can be performed simultaneously, or one anchor driver 410 can be driven prior to driving the second anchor driver 410. Still further, although this embodiment refers to two drivers 410 and two anchors 440, it should be noted that the tool 400 and procedure could be modified to use only one driver 410 and one anchor 440, or more than two drivers 410 and more than two anchors 440 without departing from the scope of the present invention.

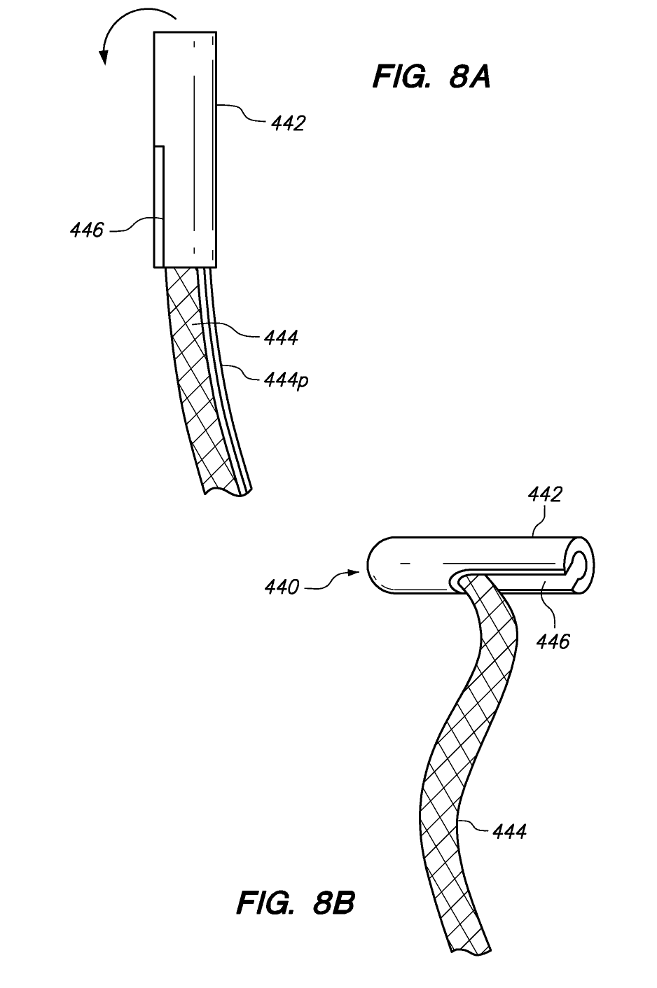

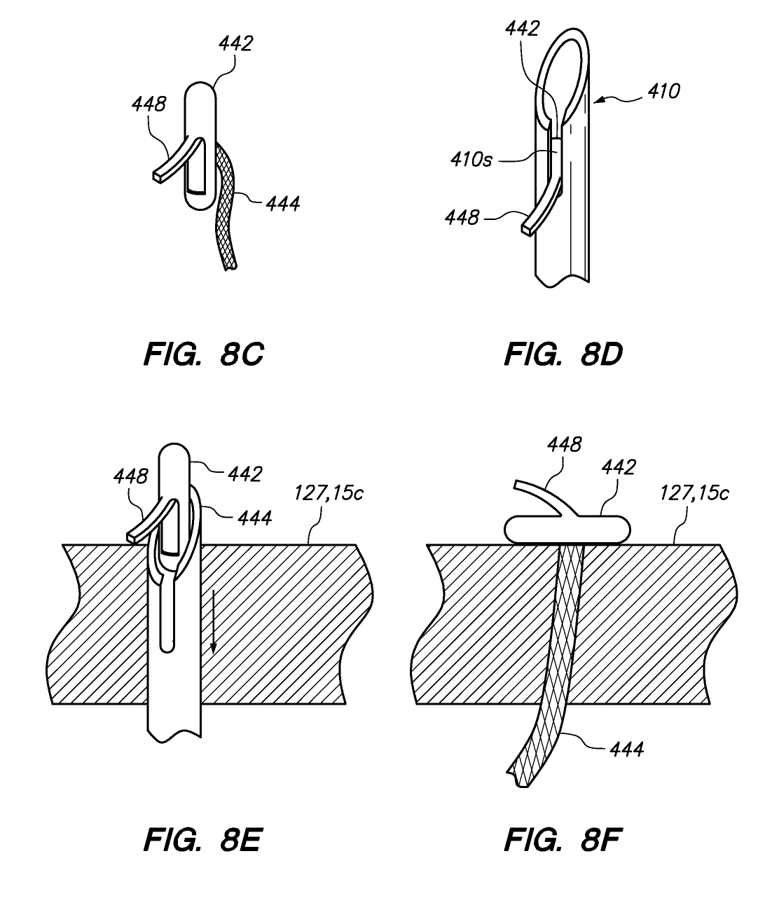

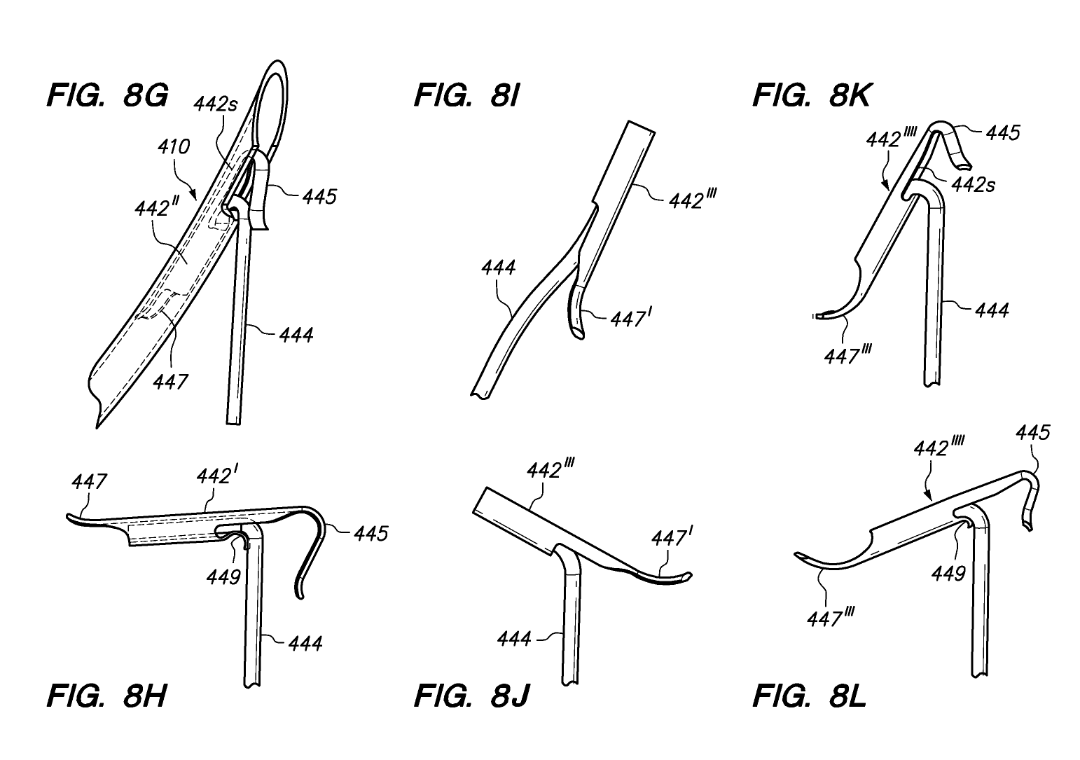

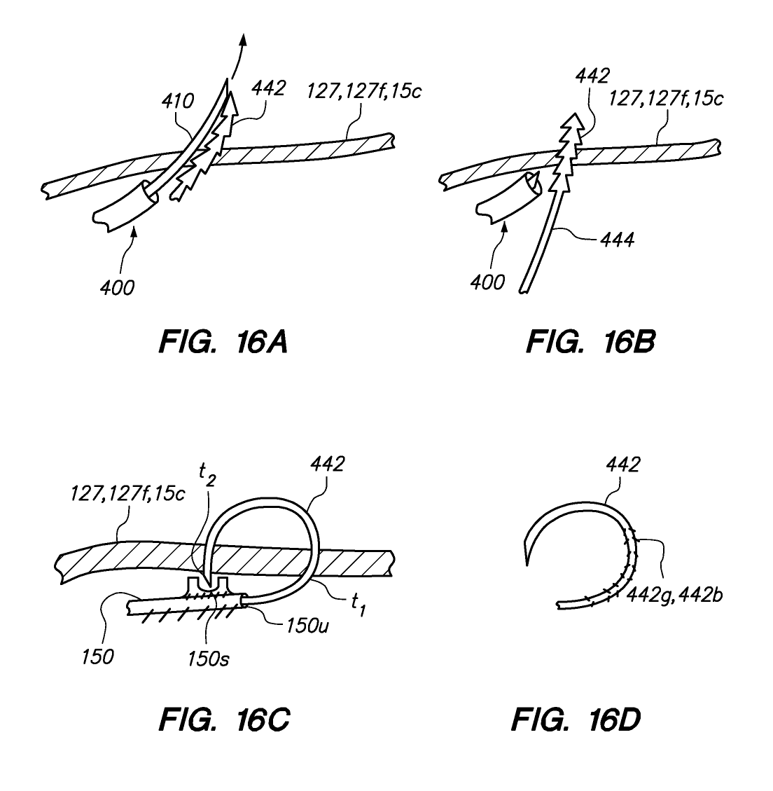

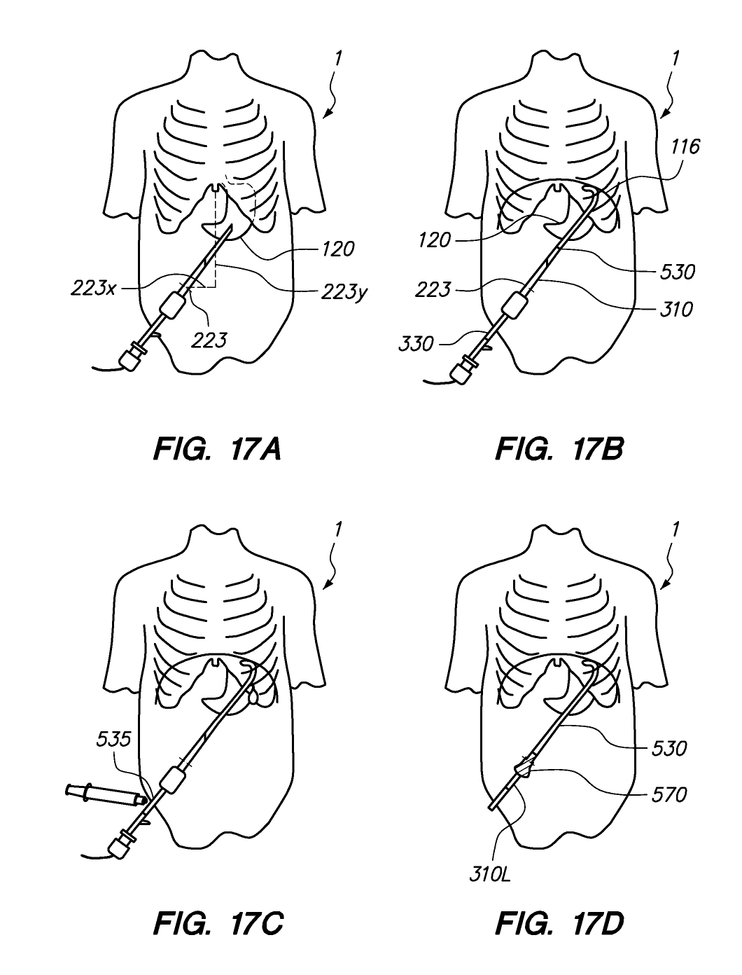

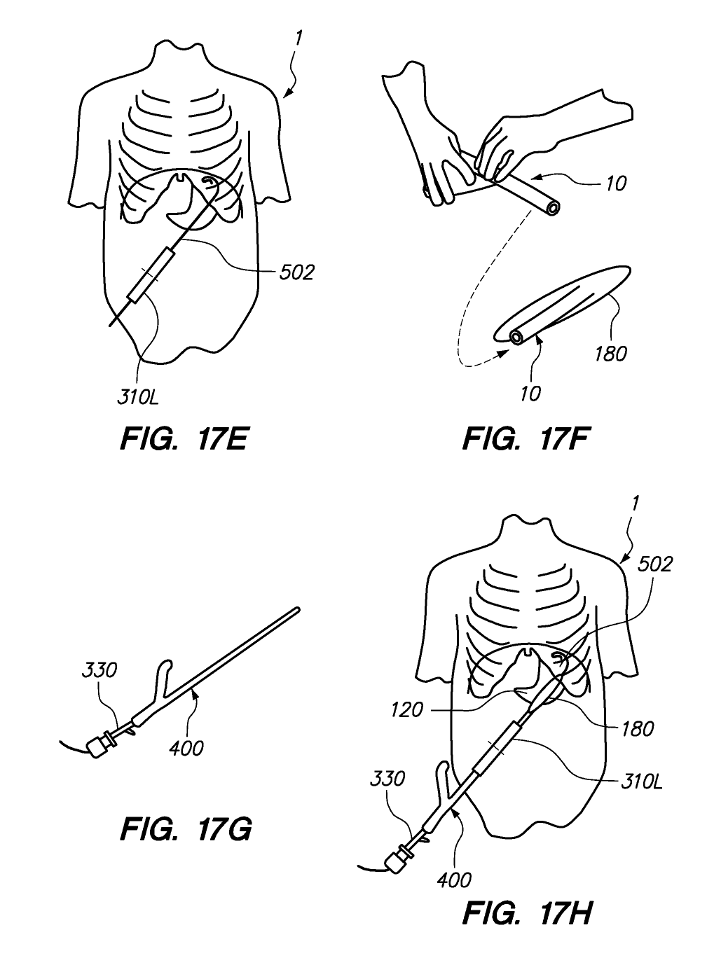

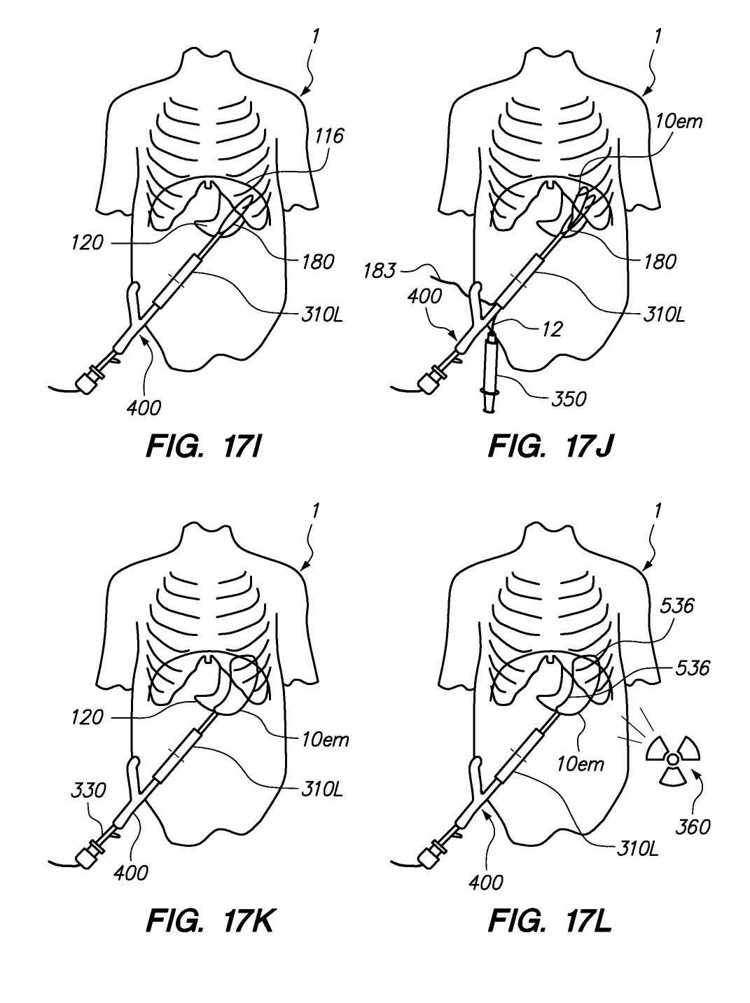

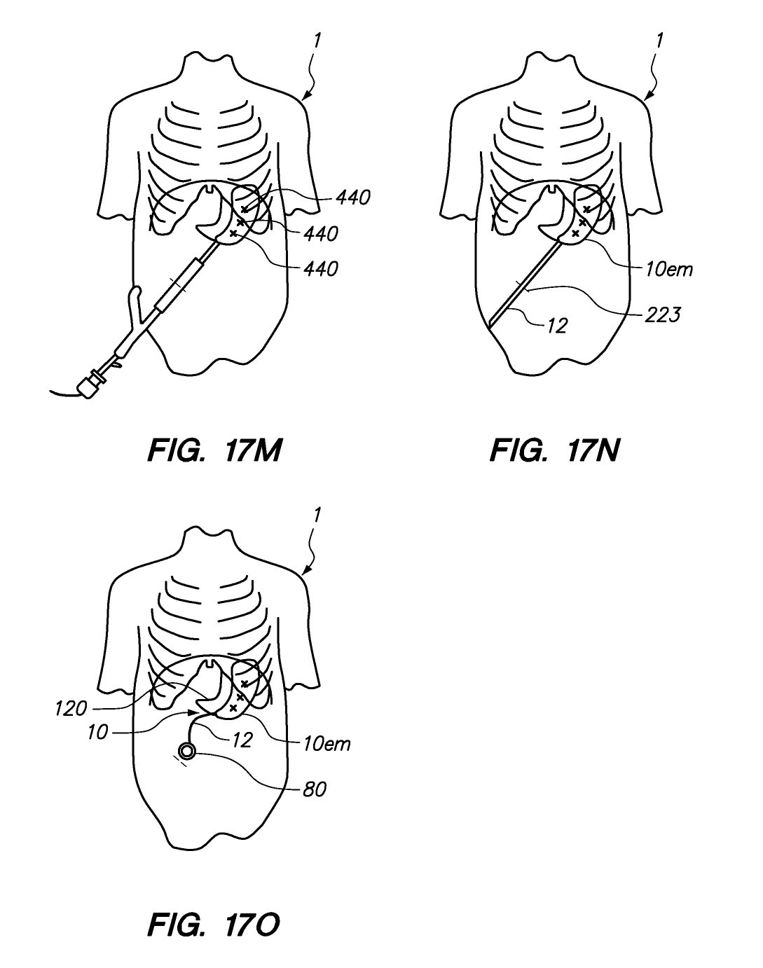

In the embodiment shown in FIG. 6K, anchor 440 comprises a T-bar 442 connected to a suture or ribbon 444 that extends proximally therefrom through the lumen 430 and out of the proximal end of the driver 410 and tool 400. However, other forms of anchor 442 may be alternatively employed, some examples of which are described below with regard to FIG. 16A-16D. FIGS. 8A and 8B illustrate one embodiment of a T-bar that is made from a rigid tube (metal or rigid plastic) and has a slot 446 that opens to one open end of the tube. Thus, when anchor 440 is inside of the anchor driver 410, the longitudinal axis of the T-bar 442 is aligned with the longitudinal axis of the suture or ribbon 444, as illustrated in FIG. 8B. After deployment of the anchors 440 as shown in FIG. 6K, when the T-bars 442 have passed through the costal cartilage 15c and fascia 127, respectively, the operator can pull back slightly on the sutures or ribbons 444, which causes the T-bars 444 to rotate as the suture or ribbon 444 of each T-bar 442 pull through the slot 446 so that an orientation such as that shown in FIGS. 8B and 6L is achieved.

The anchor drivers 410 can next be retracted back within the shaft of tool 400 to a configuration like that shown in FIG. 7A, while anchors 440 remain in place. Tool 400 is then removed from cannula 310 and at the same time removed off of sutures/ribbons 444 and guidewire 502 (in embodiments where tool 400 had been passed over guidewire 502), leaving the anchors 440 in place, as illustrated in FIG. 6M. Once tool 400 has been removed, sutures/ribbons 444 are drawn proximally by the operator by pulling on proximal portions of the sutures/ribbons 444 that extend out of the patient, to draw the T-bars 442 (or other anchor features) into engagement (contact) with the costal cartilage 15c and fascia abdominal muscle 127, respectively.

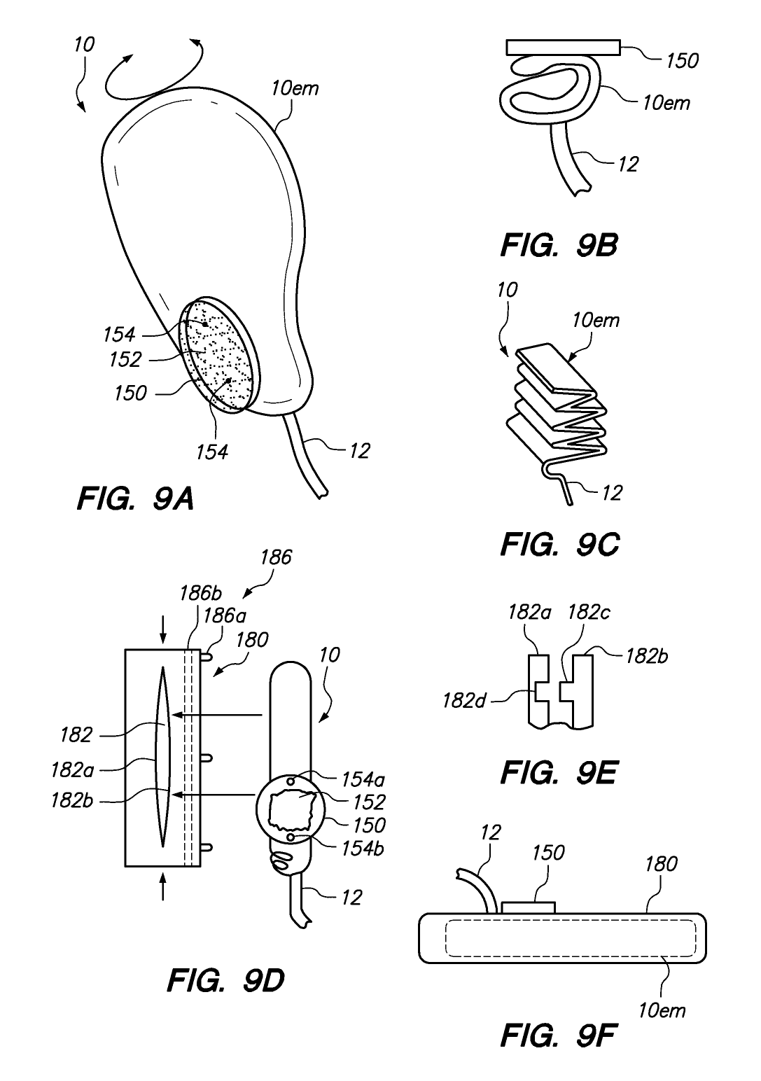

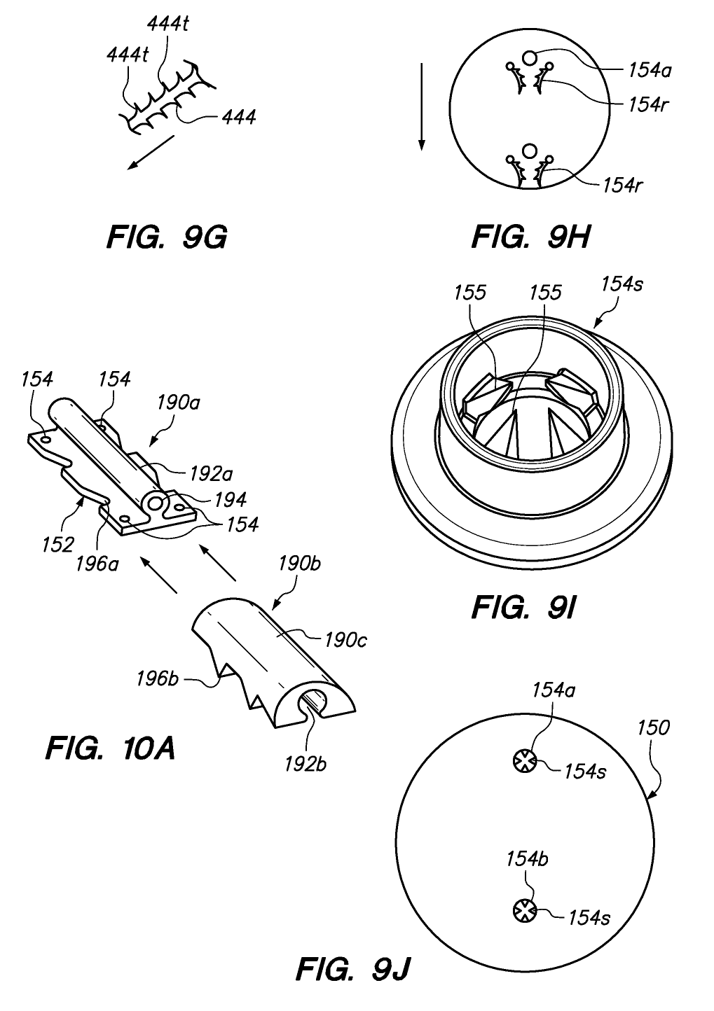

At this stage, device 10 is next inserted. Device 10 is provided in a compact configuration. For example, device 10 may be rolled (FIG. 9B) about its longitudinal axis or folded (FIG. 9C) and installed in a pod or “cocoon” 180 that maintains it in the compact configuration during delivery. Pod 180 includes a longitudinal opening 182 formed therein having sufficient length to receive device 10 in the compact configuration. The lips 182a, 182b of opening 182 may be releasably connectable, to close the opening 182 for maintaining device in the compact configuration within sheath 180, while being releasable to allow insertion of the compact device 10 into sheath 180, as well as to allow device 10 to exit sheath 180 as will be described in more detail below. Alternatively, lips 182a, 182b may be biased toward the closed configuration by the elastic properties thereof. Releasably connectable features 182a, 182b may take on various configurations. In one embodiment features 182a, 182b function like a zip lock on a sandwich bag, with male 182c and female 182d interlocking (zipping) parts, as shown in the schematic illustration of FIG. 9E. Pod 180 is made of a soft, biocompatible polymer, such as silicone, material comprising silicone, or other soft biocompatible polymer. FIG. 9D illustrates insertion of a device 10 in a compact configuration into pod 180. The device has been compacted from an embodiment of device 10 shown in FIG. 9A. Attachment tab 150 is not compacted, so that when the releasably closable features are secured, attachment tab 150 extends out of pod 180 through slot 182 as illustrated in the side view of FIG. 9F. Alternatively, the attachment tab 150 may be retained inside the pod 180 as well, with a distal end portion of the tool inserted through an opening in the proximal end portion of the pod and attaching to the attachment tab, and with the pod 180 forming a zip-lock like enclosure around this arrangement. Fill tube 12 extends out from slot 182 and the releasable closure features (e.g., zip-lock like features) close around it. Attachment tab 150 may be provided with one or more patches 152 of tissue ingrowth enhancing material, such as an expanded polytetrafluoroethylene, polytetrafluoroethylene, polyester, etc, in felt or velour or mesh configuration, or polypropylene mesh or silicone mesh, for example, so that when placed in contact with tissue, tissue is encouraged to grow into the patches.

The sutures or ribbons 444 extending from anchors 442 are threaded through the openings 154 in attachment tab 150. In the embodiment shown in FIG. 9D and FIGS. 6M-6N, the suture 444 extending from the T-bar 442 that was passed through the costal cartilage 15c is threaded through opening 154a and the other suture 444 is threaded though opening 154b. However, it is again noted here that the present invention is not limited to the use of only two anchors 444, tow sutures or ribbons 442 and two openings 154, as more or fewer could be used. The sutures or ribbons 444 and openings 154 are configured so that the sutures or ribbons 444 can only pass in one direction through the openings 154, i.e. in a proximal direction relative to openings 154. In one embodiment, sutures 444 are provided with ratchet teeth 444t (see FIG. 9G) configured to pass through opening 154 when suture 444 is drawn in the proximal direction illustrated by the arrow in FIG. 9G. The enlarged portions of the teeth 444t compress somewhat to pass through the opening 154, but then expand back to the original dimensions after passing therethrough. Thus, the enlarged distal portions of the tooth 444t abuts against the perimeter of the opening 154 and does not allow the suture to move distally through opening 154. As another example, attachment tab 150 may be provided with a ratchet mechanism 154r in connection with each opening 154 as illustrated in the underside view of attachment tab 150 (side facing fillable member 10em) FIG. 9H that allows a suture or ribbon or other tether material like an ingrowth mesh or velour 444 to pass proximally relative to the ratchet mechanism 154r, in the direction of the arrow shown, but wherein the pivoting arms for the ratcheting mechanism pivot against the suture and the teeth of the ratcheting mechanism 154r bite into the suture or ribbon 444 preventing it from moving in a distal direction relative to the ratchet mechanism 154r. In still another embodiment a speed nut 154s such as shown in FIG. 9I (showing the proximal surface of speed nut 154s) may be snapped into or press fit into each of openings 154. Alternatively, speed nuts 154s may be integrally formed in openings 154 as illustrated in FIG. 9J. Speed nut 154s is provided with multiple barbs 155. Whether speed nut 154s is made of metal or polymer or some combination thereof, barbs 115 are angled toward the distal opening of the speed nut 154s. This allows the suture or ribbon 444 to slide distally relative to speed nut 154s. However, if suture or ribbon 444 attempts to slide proximally relative to speed nut 154s, barbs 155 pierce into the suture or ribbon 444 preventing such movement. Alternatively, the speednut features could take the embodiment of a metal hypotube with teeth in the wall thickness that are bent inwards, into the annulus of the tube. These teeth allow the suture to pass through the hypotube in the direction that the teeth point towards. However, if the suture reverses direction, the teeth dig into the suture braid and halt the reverse motion of the suture.

Pod 180 is provided with a guide feature 186 for guiding pod 180 over the guidewire 502. For example, pod 180 may include external eyelets 186a that can be threaded over guidewire 502, or a lumen 186b that allows guidewire 502 to be passed therethrough. In any case, sutures or ribbons 444 are passed through the openings 154 as described above, and pod 180 (containing device 10 in a compressed configuration as described above) is passed over guidewire 502.

Alternatively, device pod 180 containing device 10 can be mounted on tool 400 and delivered thereby similar to the manner described with the procedure described in FIGS. 17A-17O below. In either case, sutures or ribbons 444 and guidewire 502 extend proximally out of the patient 1 even after the device 10 has been delivered to the target location. Upon delivering the device 10 to the target location, the fillable member is filled with liquid to an extent to cause it to break out of pod 180, as in the manner shown in FIG. 17J. When it has been visually confirmed that the device 10 is placed in the desired orientation and location, tool 400 is actuated to drive drivers 410 through the attachment tab 150, costal cartilage 15c and fascia 127f and the attachment tab 150 is anchored to the costal cartilage 15c and fascia 127f by fixing the sutures 444 against the attachment tab 150 so as to hold it against the fascia 127f and costal cartilage 15c. Fixation of the sutures may be by knots, speed nuts or other fixation mechanism. Excess suture material extending proximally of the fixation points is then cut off and removed from the patient, tool 400 is detached from device 10/attachment tab 150 and removed from the patient and guidewire 502 is also removed from the patient.

As another alternative, self-locking features of the sutures or ribbons 444, together with openings 154, that prevent distal sliding of the sutures or ribbons 444 relative to openings 154 may be used together with the anchors (e.g., T-bars) 442 to anchor the attachment tab 150 to the target location. The portions of sutures or ribbons 444 that extend proximally of attachment tab 150 can be severed and removed from the patient.

Pod 180 may be tethered to tool 400 to allow removal thereof by withdrawing tool 400. Visual inspection via an endoscope inserted through cannula 310 or fluoroscopy, or both, can be used to verify the correct location and anchoring of device 10. When it is considered that device has been satisfactorily anchored, guidewire 502 can also be removed.

Fillable member 10em of device 10 can next be further filled, if needed, to visualize the positioning and orientation of device 10. Fillable member 10em is filled by attaching a source of pressurized fluid to the open proximal end of fill tube 12 that extends out of the patient 1. In at least one embodiment fillable member 10em is filled to an initial volume at this stage. Viewing can be performed endoscopically and/or fluoroscopically. Once there is satisfaction that device 10 has been properly placed and attached, such as by visual verification, an access member 80 can be installed according to any of the techniques described herein or in any of the co-pending application Ser. Nos. 11/407,701; 11/881,144; 11/716,985; 1/716,986 and 11/974,444 incorporated herein. Access member can be installed through the same minimally invasive opening 223 and delivery tract through which the previous procedures were performed. Alternatively, the opening through the fascia along the delivery tract may be closed down around the fill tube 12, and access member 80 can be fixed to the fascia 127f or abdominal muscle 127 in a location adjacent to or removed from the closed opening. FIG. 6O shows a frontal view of the access member 80 fixed to the fascia abdominal muscle 127 of the patient 1. The fat 131 and skin 125 can then be closed over the access member to complete the procedure. The side view of FIG. 6P illustrates the tissues that overlie the access member 80. In this example, the location of access member 80 on the midline axis 110 of the patient 1 makes it easier to locate after it is implanted, should access member 80 need to be accessed to increase or decrease the volume of the device 10 by inputting or drawing out a volume of fluid via access member 80.

Referring again to FIG. 7A, this embodiment of anchor delivery tool 400 includes an axially aligned (horizontal) handle 412a and a transverse (vertical) handle 412t. By pulling (proximally) back on the transverse handle 412t while holding handle 412a and using it to provide stabilization and maintain the position of tool 400 relative to the body of the patient in the proximal-distal axial direction, this provides excellent leverage to maintain good contact between the portions 404, 406 of tool 400 with costal cartilage 15c as members 410 are deployed through the costal cartilage 15c and fascia/abdominal muscle. In this embodiment, actuator 416 is provided on transverse handle 416 to allow the operator to actuate actuator 416 with the same hand/fingers used to manipulate handle 412t. FIG. 7D shows an alternative embodiment of tool 400 that differs from the embodiment of FIG. 7A only in that actuator 416 is provided to extend from axial handle 412a rather than transverse handle 412t. Since more force/control may need to be provided through handle 412t relative to that provided through handle 412a, the provision of actuator 416 so as to be actuated using the hand that operates handle 412a, allows the hand operating handle 412t to focus all of its strength and control on the handle manipulation function. FIG. 7E shows an alternative embodiment of tool 400 that differs from the embodiment of FIG. 7D only in that actuator 416 is provided as an axially aligned twist grip rather than a trigger. By providing the rotating actuator 416 of FIG. 7E, there is no risk of the actuator extending up against the patient's body. Like the embodiment of FIG. 7D this allows the hand operating the transverse handle 412t to be dedicated to stabilization/control functions. The axial handle 412a can also be used to provide stabilization at the same time that actuator 416 is operated, similar to the way that a motorcycle rider can operate the twist grip throttle while also steering/stabilizing the motorcycle. The rotational actuation also allows good positive control over both advancing and retracting the actuation of components 410 as needed.

FIG. 7F illustrates a visual indicator 428 that identifies to the user at least the relative amount of deployment of the anchor drivers 410. In FIG. 7F, actuator indicator includes a graphical scale with a gradually increasing thickness that graphically illustrates the relative amount of deployment of drivers 410, with the thinner end indicating no deployment and the thicker end of the graphical bar illustrating full deployment when aligned with an indication arrow on the non-rotating part of handle 412a. Alternatively, the scale may include numbers, e.g., fractions to show the relative amount of deployment or numbers to show the actual deployment distances. Tools 400 having trigger-style actuators 416 may also be configured with a visual indicator to show the actual or relative amount of deployment of drivers 410 by actuator 416. By reverse motion of actuator 416, drivers 410 can be driven in the reverse direction, i.e., proximally. Accordingly, drivers 410 can be retracted to reposition tool 400 if needed. Also, drivers 410 can be fully retracted prior to removing tool 400 after the delivery of the anchors has been successfully completed.

Once the anchors (such as T-bars) 442 are driven through the tissues that device 10 is to be anchored to and released, the anchor drivers (such as needles) 410 no longer need to be deployed. The user can then manually retract the drivers 410 as described above. Alternatively, drivers 410 may be configured to automatically retract (such as with a coil driven action) after the anchors have been deployed and the user releases actuator 416. This could be accomplished, for example, by spring-biasing the actuator (whether twist grip style, trigger style or other embodiment) towards the undeployed configuration.

Alternatively, actuator 416 may be configured to automatically release from driving the anchor drivers 410 when actuator is driven past the full deployment position. FIG. 7G shows such an arrangement where on the visual indicator 428, an automatic retraction indicator 430 is indicated after the actuator 416 fully deploys the drivers (indicated at 428f) and then actuator 416 is rotated past the full deployment orientation to release from the drivers 410 as indicated by alignment of the pointer 429 with indicator 430. Drivers 410 in this case are spring-biased to retract back into tool 400 when no driving forces are applied thereto by actuator 416. It is preferred that full deployment of drivers 410 is achieved by less than a full 360 degree rotation of actuator 416 relative to handle 412a. The drivers 410 and portions of the tool around openings 420 are preferably radiopaque for visualization under fluoroscopy. Portion 404 should also be radiopaque.

FIG. 7H shows still another alternative embodiment of tool 400 that differs from the previous embodiments only in that actuator 416 is provided as a switch or slider 416 that can be slid relative to slot 416s in handle 412a in order to deploy or retract drivers 410. Any or all of the other optional features (e.g., visual indicator 428, automatic retraction of drivers, etc.) can also be provided with this embodiment.

FIG. 7I illustrates another embodiment of tool 400, which may employ any of the different configurations of actuator 416 described above, as well as any or all of the other optional features described above. In this embodiment transverse handle 412t is movable to a stowed configuration, where handle 412t is substantially aligned with axial handle 412a as shown in FIG. 7I. This facilitates ease of manipulation of tool 400 as it is manipulated into position to engage the distal end portion 418 with the costal cartilage 15c as described above, so that handle 412t does not form an obstruction during any of these maneuvers.

FIG. 8C illustrates an alternative embodiment of T-bar 442 to that described above with regard to FIGS. 8A-8B. In the embodiment of FIGS. 8A-8B, T-bar 442 is ejected from the distal end of needle 410 by pushing on the proximal end of T-bar 442 with a pusher 444p inside the driver 410. This is referred to as active deployment of an anchor. The embodiment of FIG. 8C is passively deployable. T-bar 442 in FIG. 8C includes a tang 448 that angles out away from the main body portion of T-bar 442 in a direction from the distal end of the T-Bar 442 to the proximal end of the T-bar, as oriented in the driver 410. Driver 410, such as a needle with a lumen therethrough is provided with a distal slot 410s so that when T-bar 442 is inserted into needle 410, tang 448 extends from slot 410s, as illustrated in FIG. 8D. Accordingly, when drivers 410 are driven through the tissues to which device 10 is to be attached, such as the fascia abdominal muscle and the costal cartilage 15c (FIG. 7C) or the like, and then drivers 10 are either manually or automatically retracted, as described above, as the driver/needle retracts, tang 448 catches against the tissues that the needle/driver is retracting back though, as illustrated in FIG. 8E. Once driver 410 has been completely retracted from the tissue 127 or 15c, proximal retraction on suture 444 causes T-bar 442 to assume an orientation transverse, perpendicular or nearly perpendicular to suture 444, as illustrated in FIG. 8F, thereby providing the most anchoring leverage against the tissue 127 or 15c, etc.

FIGS. 8G-8H illustrate another embodiment of a passively deployable anchor arrangement 440 including T-bar 442′ and suture 444. FIG. 8G shows T-bar 442′ mounted or received in the distal end portion of an anchor drive 410, in this case, a slotted needle 410. Extending from the distal end of the main body 442′ is a hook 445 that also extends out of the slot 410s of driver 410 when the main body is mounted in the lumen of the driver/needle 410. In this embodiment, suture 444 also extends out of the slot 410s of driver 410, at a location proximal to the location where hook 445 extends out of slot 410s. A radiused suture support 449 is provided for an atraumatic surface where the suture 444 bends around and contacts T-bar 442′, thereby preventing cutting of the suture 444 as tension is applied to reorient the T-bar 442′. A slot 442s is provided in T-bar 442′ where it transitions from the main body to the hook 445. Slot 442s guides the suture 444 into the main body of the T-bar 442′ and also helps maintain alignment of the suture 444 against the support 449.

Neither tool 400 nor driver 410 needs to be withdrawn over the suture after driving the T-bar 442′ through the target tissue. Rather, upon retraction of the driver 410 out of the tissue, hook 445 catches on the tissue, thereby maintaining the T-bar 442′ and suture 444 relatively stationary as the driver 410 is withdrawn. This completely frees the anchor 440, both T-bar 442′ and suture 444 from the driver 410. Accordingly, neither tool 440 nor driver 410 needs to be completely removed from the patient, as the suture is not threaded through either of these components. After passive deployment of the anchor 440, tension on suture 444 assists in reorienting the T-bar 442′ to a more transverse orientation relative to suture 444. An example of such an orientation is shown in FIG. 8H. However, a “transverse orientation” is not limited to ninety degrees relative to the suture 444, but may be a smaller or larger angle, e.g., about 45 degrees to about 135 degrees, or about 60 degrees to about 120 degrees.

A tail portion 447 extends proximally from the main body portion of T-bar 442′. Although the proximal tip of tail 447 may taper slightly radially outward from the radius of the cylindrical formation of the main body of T-bar 442′, this is for purposes of making the tip more atraumatic, and is optional. Thus, tail 447 can extend proximally without extending radially outwardly past the outer surface of the main body of T-bar 442′. In either case, tail 447 is configured to be received within the lumen of the driver/needle 410, as shown in FIG. 8G. Therefore, the radial extent of the proximal tip must be such that a distance from the proximal tip, to a diametrically opposed location that is an axial extension of the main body of T-bar 442′, in a plane perpendicular to the longitudinal axis of the main body of T-bar 442′, is less that the inside diameter (lumen diameter) of needle 410. Upon reorientation of T-bar 442′ as shown in FIG. 8H for example, tail 447 provides additional leverage/resistance to improve the anchoring function of the T-bar 442′. Alternatively, the tail 447 can be manufactured so that it extends radially outwards beyond the outer diameter of the body 442′. In this embodiment, the tail would be deformed back to the diameter of the body when the T-bar 442′ is assembled into the driver 410. This deformation could be completely or partially an elastic deformation such that when the T-bar 442′ is released from the driver 410, the tail 447 springs radially outwards and takes a geometry that further encourages rotation of the T-bar 442′.

FIGS. 8I-8J illustrate another embodiment of a passively deployable anchor arrangement 440 including T-bar 442′″ and suture 444. FIG. 8I shows T-bar 442′″ in the orientation that it assumes when received in a slotted needle 410. In this embodiment suture 444 is threaded outside and alongside of the needle 410, and tail 447′ extends outwardly and proximally of slot 410s of needle 410. Tail 447′ may be curved radially outwardly at least a proximal end portion thereof to enhance the ability of the tail 447′ to catch against tissue as needle 410′ is withdrawn, thereby passively deploying anchor 44. Once needle 410 has been removed, tension on suture 444 and the abutment or catching to tail 447′ against tissue act together to reorient T-bar 442′″ in a transverse orientation relative to suture 444 as illustrated in FIG. 8J.

A slot is provided in T-bar 442′″ where it transitions from the main body to the tail 447′. The slot guides the suture 444 into the main body of the T-bar 442′″ and helps to maintain it in alignment with the tail when anchor 440 is received in driver 410.

FIGS. 8K-8L illustrate another embodiment of a passively deployable anchor arrangement 440 including T-bar 442″″ and suture 444. The embodiment of FIGS. 8K-8L is similar to the embodiment of FIGS. 8G-8H in that it has a hook 445 extending from the distal end of the main body 442″″ that also extends out of the slot 410s of driver 410 when the main body is mounted in the lumen of the diver/needle 410. Also similarly, suture 444 also extends out of the slot 410s of driver 410, at a location proximal to the location where hook 445 extends out of slot 410s. A radiused suture support 449 is also provided as an atraumatic surface where the suture 444 bends around and contacts T-bar 442″″, thereby preventing cutting of the suture 444 as tension is applied to reorient the T-bar 442″″. A slot 442s is provided in T-bar 442″″ where it transitions from the main body to the hook 445. Slot 442s guides the suture 444 into the main body of the T-bar 442″″ and also helps maintain alignment of the suture 444 against the support 449.

However, the embodiment of FIGS. 8K-8L differs from the embodiment of FIGS. 8G-8H by the proximal tail portion 447′″ that is provided. Whereas tail portion 447 of T-bar 442′ is provided along the same side of the main body 442′ from which hook 445 extends, tail portion 447′″ is provided to extend from a side of the main body 442′″ that is diametrically opposite of the side from which hook 445 extends. Further, tail 447′″ does not extend radially outwardly from the side where it originates, but rather extends in a direction that is radially inward from the side where it originates. The proximal tip of tail 447′″ may optionally extend to a radial extent such that a distance from the proximal tip, to a diametrically opposed location that is an axial extension of the main body of T-bar 442″″ on the side that the tail 47′″ originates from, in a plane perpendicular to the longitudinal axis of the main body of T-bar 442″″, is less that the inside diameter (lumen diameter) of needle 410. Upon reorientation of T-bar 442″″ as shown in FIG. 8L for example, tail 447′″ provides additional leverage/resistance to improve the anchoring function of the T-bar 442″″. Alternatively, the tail 447′″ can be manufactured so that it extends radially outwards beyond the outer diameter of the body 442″″. In this embodiment the tail would be deformed back to the diameter of the body when the T-bar 442″″ is assembled into the driver 410. This deformation could be completely or partially an elastic deformation, such that when the T-bar 442″″ is released from the driver 410, the tail 447′″ springs radially outwards and takes a geometry that further encourages rotation of the T-bar 442″″.

Neither tool 400 nor driver 410 needs to be withdrawn over the suture after driving the T-bar 442″″ through the target tissue. Rather, upon retraction of the driver 410 out of the tissue, hook 445 catches on the tissue, thereby maintaining the T-bar 442″″ and suture 444 relatively stationary as the driver 410 is withdrawn. This completely frees the anchor 440, both T-bar 442″″ and suture 444 from the driver 410. Accordingly, neither tool 440 nor driver 410 needs to be completely removed from the patient, as the suture is not threaded through either of these components. After passive deployment of the anchor 440, tension on suture 444 assists in reorienting the T-bar 442″″ to a more transverse orientation relative to suture 444. An example of such an orientation is shown in FIG. 8L.

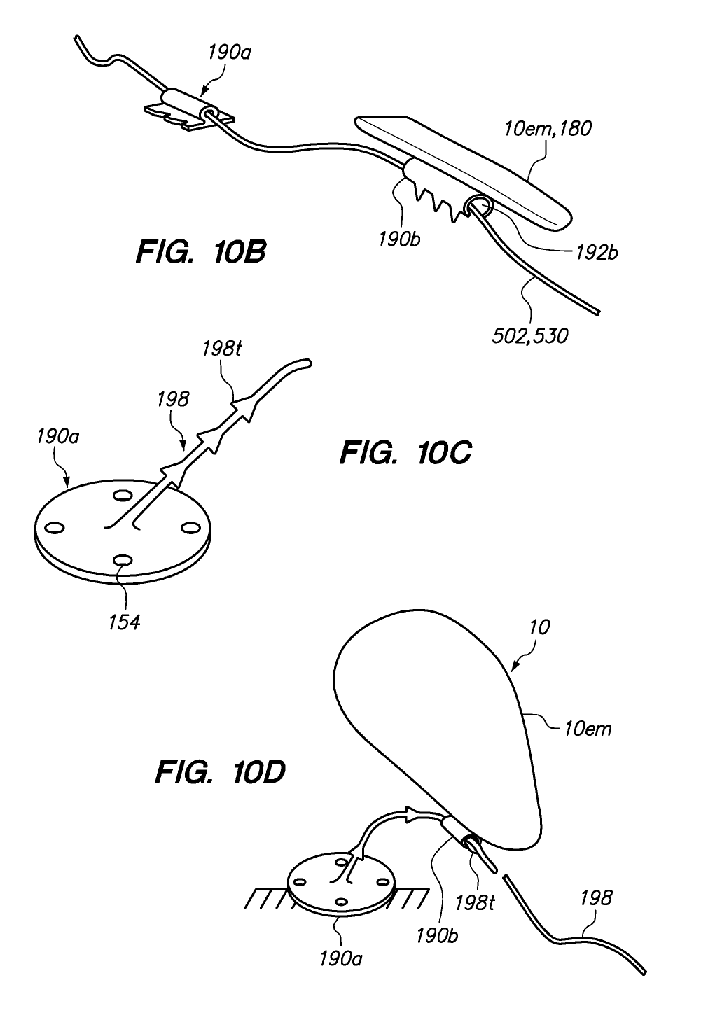

FIG. 10A illustrates an alternative mechanism for anchoring device 10 in the abdominal cavity. In this embodiment, an anchor docking mechanism 190a, 190b is provided to replace the attachment tab 150 described above. Accordingly, after fixing the anchors 442 in the desired locations (FIG. 6M), in this embodiment anchor docking member 190a (such as an anchoring rail or other anchor docking member having docking features configured to mate with and interlock with interlocking docking features provided on anchor docking member 190b) is first delivered to the target site and anchored there. To accomplish this, sutures or ribbons 444 can be threaded through openings 154, in member 190a in the same manner as described above with regard to attachment tab 150, or other anchoring or attachment members can be delivered through openings 154 to anchor it to an internal body structure. Anchor docking member 190a may further be provided with a lumen 194 through which guidewire 502 or guide 530 can be passed, so that anchor docking member 190a can be delivered over guidewire 502 or guide 530 to the target site to be anchored to. Tool 400 may be used to perform this procedure, or, alternatively, anchor docking member 190a can be pushed over the guidewire 502 or guide 530 using graspers or other endoscopic tool, for example, and once member 190a is in the desired anchoring position, sutures or ribbons 444 can be cut with a cutting mechanism on tool 400 if used, or with endoscopic scissors, or the like. Alternative anchoring features described herein may be used in place of the use of T-bars 442 and sutures or ribbons 444. Anchor docking member 190a may be provided with a tissue ingrowth enhancing material 152 on the surface configured to contact the internal body structures during anchoring, so that when placed in contact with tissue, tissue is encouraged to grow into the material 152.

Once anchor docking member has been anchored against one or more internal body structures (e.g., costal cartilage 15c and fascia/abdominal wall 127), tool 400, if used is next removed. In either case, device 10 is next provided in a compact configuration, such as described above and shown in FIG. 10B, and may optionally be retained in a pod 180 (described in more detail below). The anchor docking member 190b shown in FIG. 10B is provided as a fixed component of device 10 that replaces attachment tab 150. Alternatively, a design could be used that has an attachment tab bonded directly to the expandable member 10em. This would allow the docking mechanism to be used only as a short term means of fixation. Tissue ingrowth into the attachment tab would provide long term fixation of the expandable member 10em. The surface 190c faces fillable member 10em, and the opposite surface of member 190b interfaces and locks with anchor docking member 190a. Member 190b is provided with a mating guide member 192b that mates with guide member 192a as member 190b is slid relative to member 190a. In the example shown, member 192a is a rail and member 192b is a mating channel. However, these could be reversed so that member 190a is provided with a channel and member 190b is provided with a rail. Further alternatively, other arrangements of slidably matable members 192a, 192b could be substituted as would be apparent to those of ordinary skill in the art.

The mechanism 190a, 190b is further provided with mating, interlocking features 196a, 196b that allow member 190b to be advanced in a distal direction over member 190a, but prevent movement of member 190b relative to member 190a in a proximal direction. In the example shown, the members are provided with a series of cams or ratchet teeth 196a, 196b configured to allow movement of 190b in the distal direction but prevent movement of 190b in the proximal direction, relative to 190a.

Using this alternative arrangement device 10 is provided in a compact configuration, as noted, and guidewire 502 or guide 530 is threaded through channel 192b. Tool 400 may be used to at least partially insert the compacted device 10 therein for delivery as described above. Alternatively, the compacted device can be inserted into the cannula 310. In either case, device 10 is guided over guidewire 502 or guide 530 which aligns components 192a, 192b. Continued distal advancement of device 10 drives member 190b into engagement with member 190a as the guide components 192a, 192b slidably interact. Locking features 196a, 196b may be configured to allow positioning of device 10 at more than one location along the proximal-distal axis defining the anchor docking mechanism 190a, 190b. Once device 10 has been fixed at the desired location, there is no need to sever sutures or ribbons 444 in this embodiment at this stage, as this has already been performed. The procedure then can continue in the same manner as described above with regard to FIGS. 6A-6P.

FIGS. 10C-10D illustrate another alternative mechanism for anchoring device 10 in the abdominal cavity. In this embodiment, an anchor docking mechanism 190a, 190b is provided to replace the attachment tab 150 described above. In this embodiment, anchor member 190a includes a tether 198 having multiple ratchet features or teeth 198t that function like teeth or ratchet features 444t described above and are arranged along the tether 198, as shown in FIG. 10C. The anchor member 190a can be delivered by tool 400 or otherwise placed, with T-bars 442 and sutures/ribbons 444 or other anchoring features being applied through holes 154 to anchor member 190a to at least one internal body structure. Accordingly, after anchoring member 190a in the desired location, tether 198 has sufficient length to extend proximally out of the body of the patient through incision 223. Alternatively, the distal portion containing teeth 198t can be connected to a proximal tether portion 198 that does not include ratchet features/teeth 198t and is separable from the distal portion after the implant 10 is locked into position as desired. Docking member 190b is provided on device 10 in the same way as described above with regard to FIGS. 10A-10B. However, in this case, docking member 190b may be cylindrical, with an annulus dimensioned to allow ratchet features 198t to pass into a distal end of docking member 190b and out of the proximal end thereof, but which prevents ratchet features from passing back into the proximal end of docking mechanism 190b, once they have been drawn out of the proximal end. This allows docking member 190b to be slid distally over the tether 198 and ratchet features 198t, but prevents backsliding of docking member 190b by preventing proximal movement of docking member 190b relative to tether 198.

Device 10 can therefore be delivered in a compact configuration into the patient's body through the delivery tract formed beginning where incision 223 was made. Device may optionally be contained in pod 180 during delivery. By sliding docking member 190b distally over tether 198, docking member 190b and thus device 10 can be placed in the intended location in the abdominal cavity. Advantageously, this arrangement allows a non-rigid attachment of device 10 relative to at least one internal body structure, as tether 198 is flexible, and docking member 190b need not be drawn all the way into contact with anchoring member 190a, although it may optionally be fixed in this way. FIG. 10D illustrates an example of a non-rigid anchoring of device 10 where a length of tether 198 remains between members 190a and 190b. Note that this shows the location where device has been decided to be placed and fillable member 10em has already been filled. An excessive length of tether 198 that extends proximally of docking member 190b after completing the anchoring of device 10 at the desired location can be removed by severing.

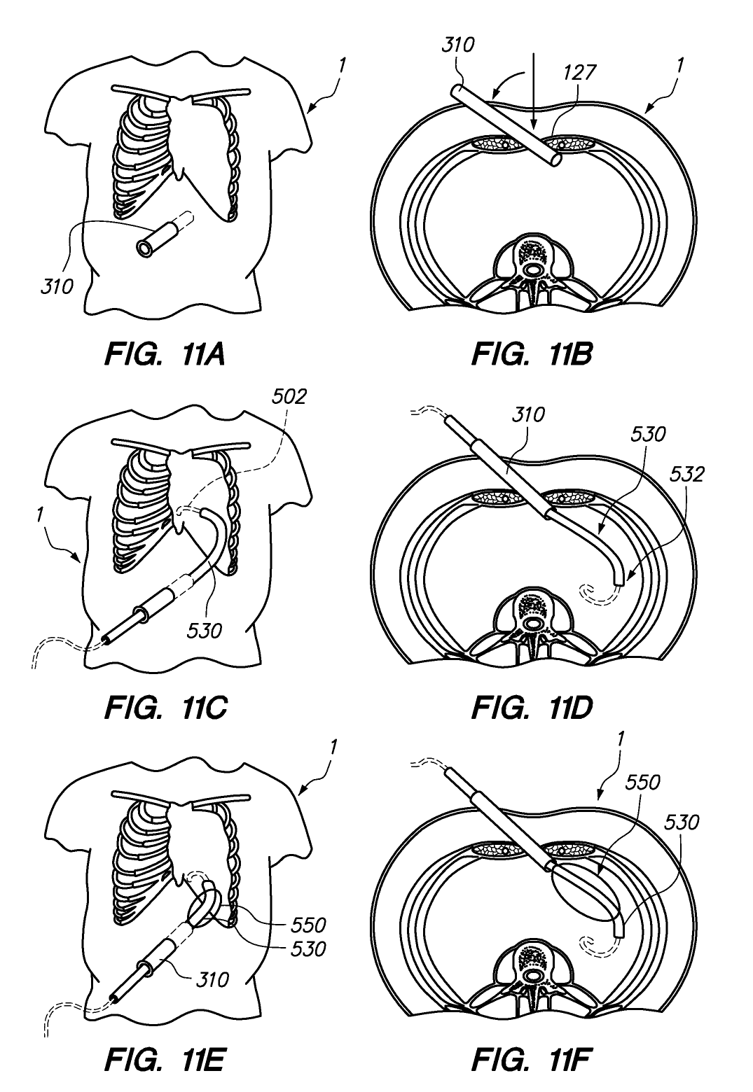

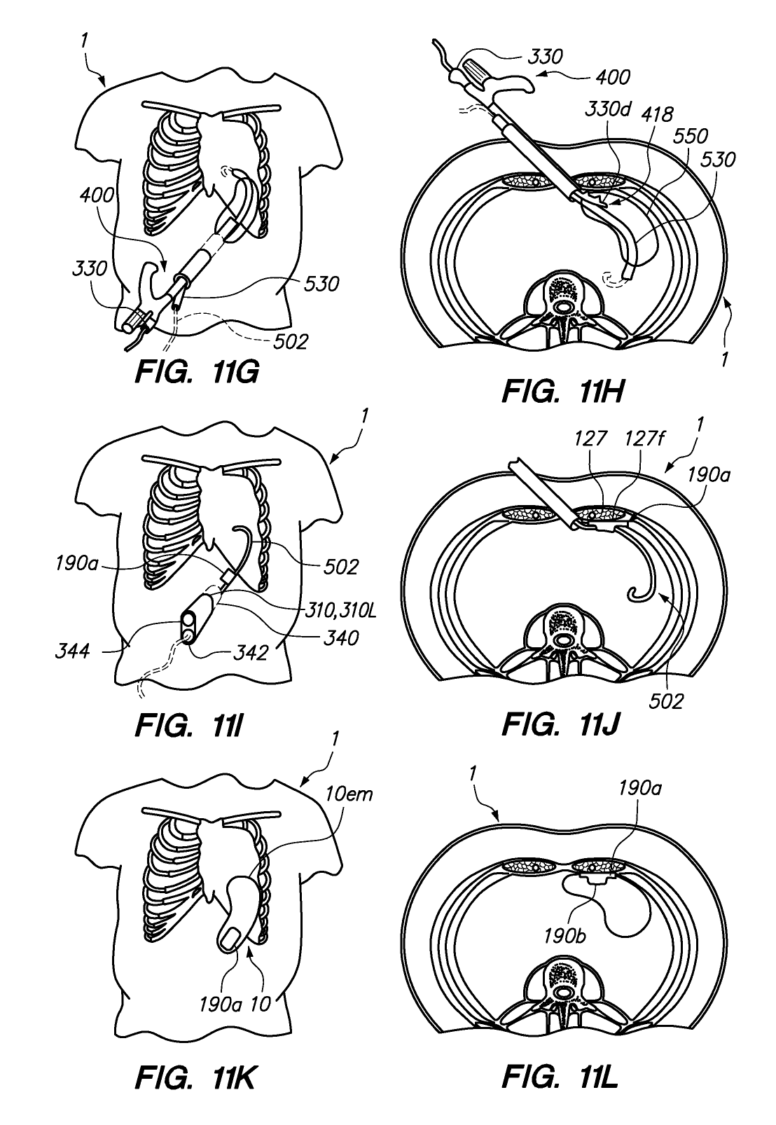

FIGS. 11A-11L illustrate various parts of other embodiments of a procedure for percutaneously implanting a fillable extra-gastric device 10 according to alternative embodiments of the present invention. After preparing the patient 1 in a manner as described above with regard to the procedure described regarding FIGS. 6A-6P, an incision 223 is made and cannula 310 is inserted into the incision and advanced in the same manner, and using the same tools as described above with regard to FIGS. 6B-6C. A delivery tract is thus formed as described above.

Once the delivery tract has been opened into the abdominal cavity, as visually confirmed via the endoscope 330, the endoscope 330 and trocar 320 are removed from the cannula 310, while leaving the cannula 310 in place through the delivery tract and extending into the abdominal cavity. The cannula 310 is then tilted, as shown in the sectional illustration of FIG. 11B, so as to point toward the location where the device 10 is to be implanted. Cannula 310 is also angled at a shallow angle relative to the skin 125 of the patient so that tools or devices inserted through the cannula 310 are directed under the costal cartilage 15c locations and up and around a pathway generally following the diaphragm 116.

Once the orientation of the cannula 310 is established as described above, a guide member 530 is inserted through cannula 310 which directs the trajectory of the distal end portion of the guide 530 up and underneath the costal cartilages 15c to ride around the curvature of the diaphragm 116. Optionally, a guidewire 502 (shown in phantom FIG. 11C) may first be inserted and directed up and underneath the costal cartilages 15c, as in the procedure described with regard to FIG. 6E, after which guide 530 can be delivered over the guidewire 502 and into position. It can be helpful to use 0.5 liters or less of carbon dioxide insufflation to create a small passage for the guide member 530 to be advanced in, as described previously. However, it is preferred not to use the guide wire 502, as guide 530 can perform all of the functions of the guidewire 502, and perform them better in some instances. For example, it is sometimes difficult to extend guidewire 502 through fatty tissues as the guidewire 502 is sometimes too flexible or “floppy” to maintain its course against the resistance of the fat. In order to firm up the resistance of the guidewire 502, a somewhat less flexible tube can be delivered over the guidewire 502 to stiffen up the guidewire by its support. Although this helps maintain the guidewire 502 on its course through the fatty tissues, the tip of the guidewire 502 is quite small and, when guidewire 502 is rigidified by the additional tube passed thereover, there is some risk of the tip of the guidewire 502 becoming supported rigidly enough to cause some trauma to internal abdominal structures.

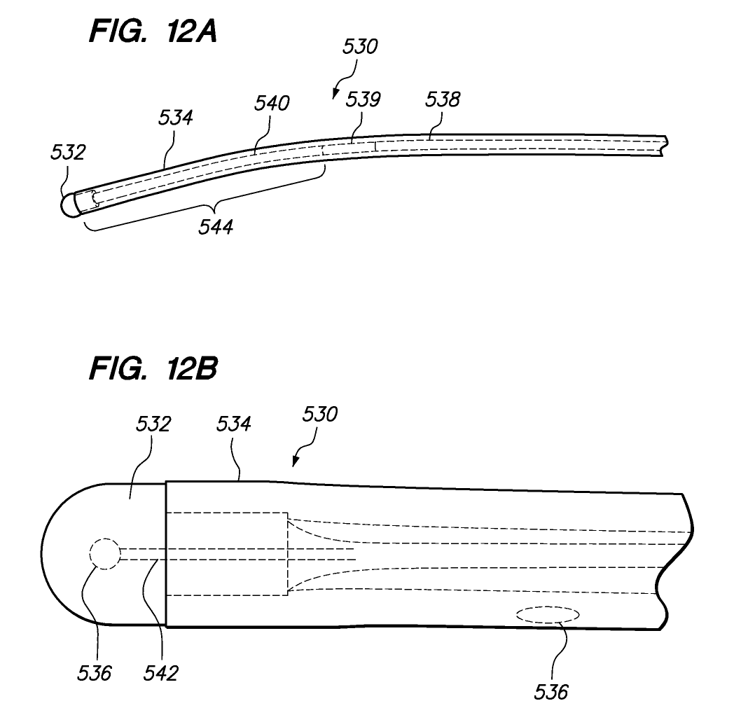

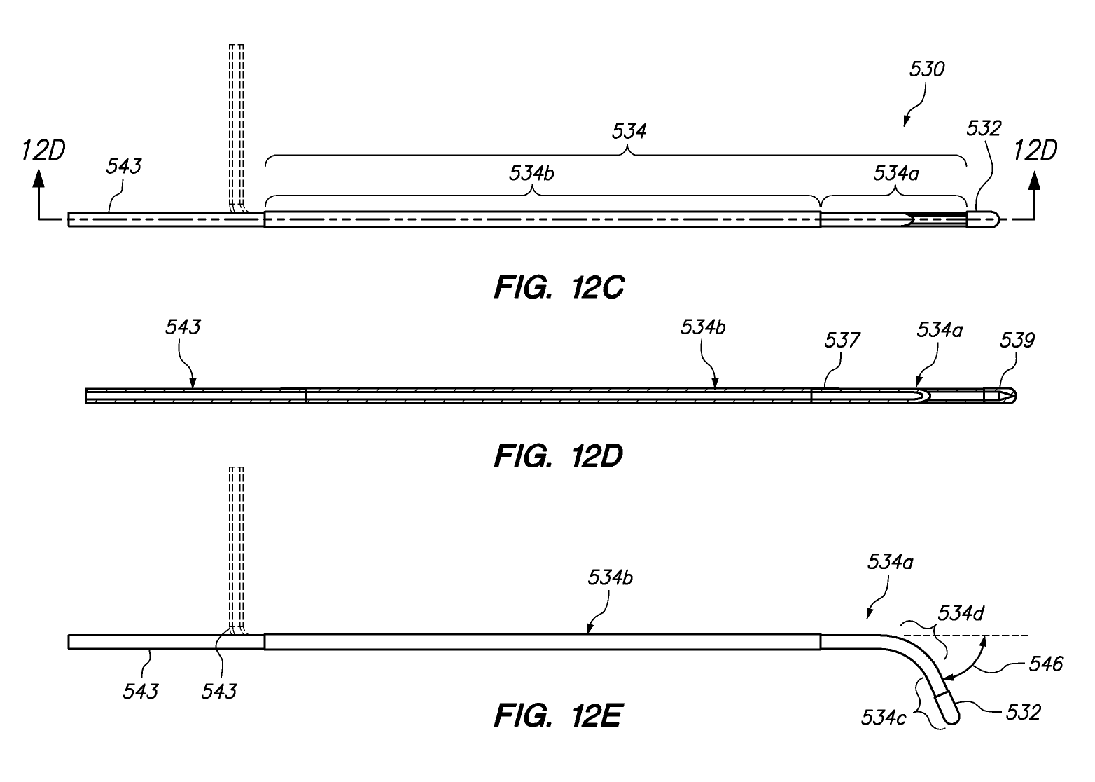

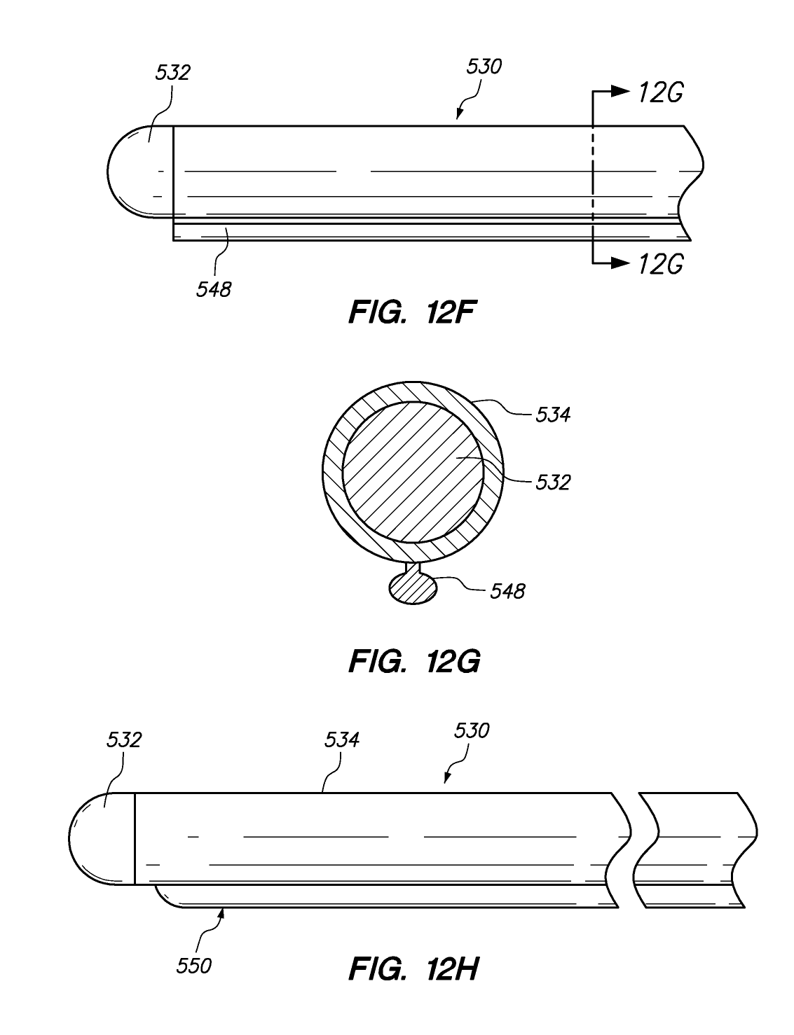

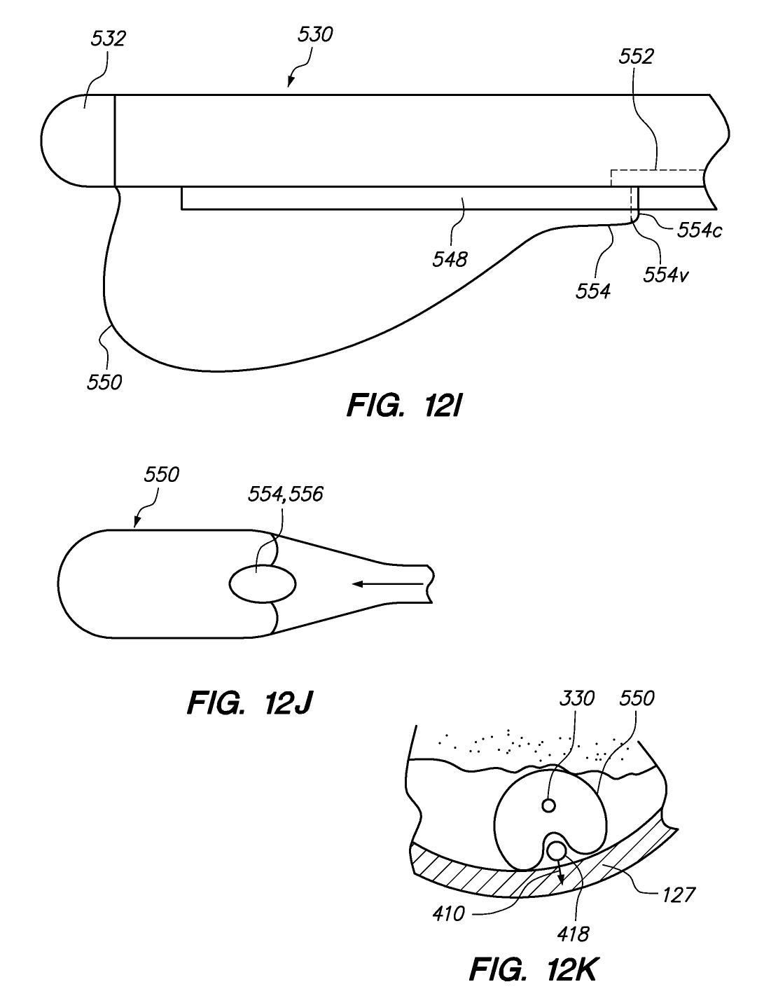

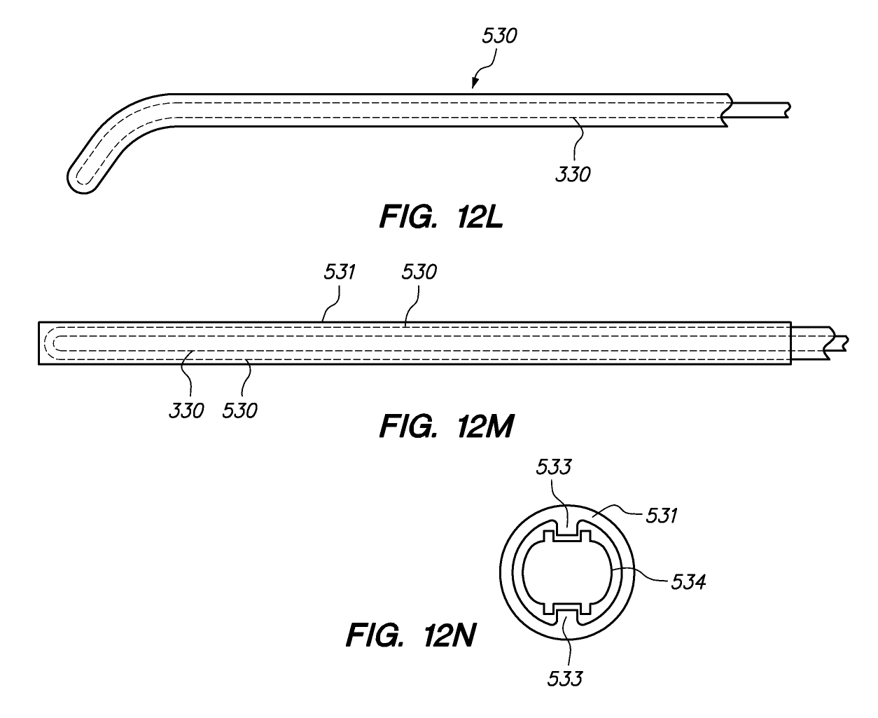

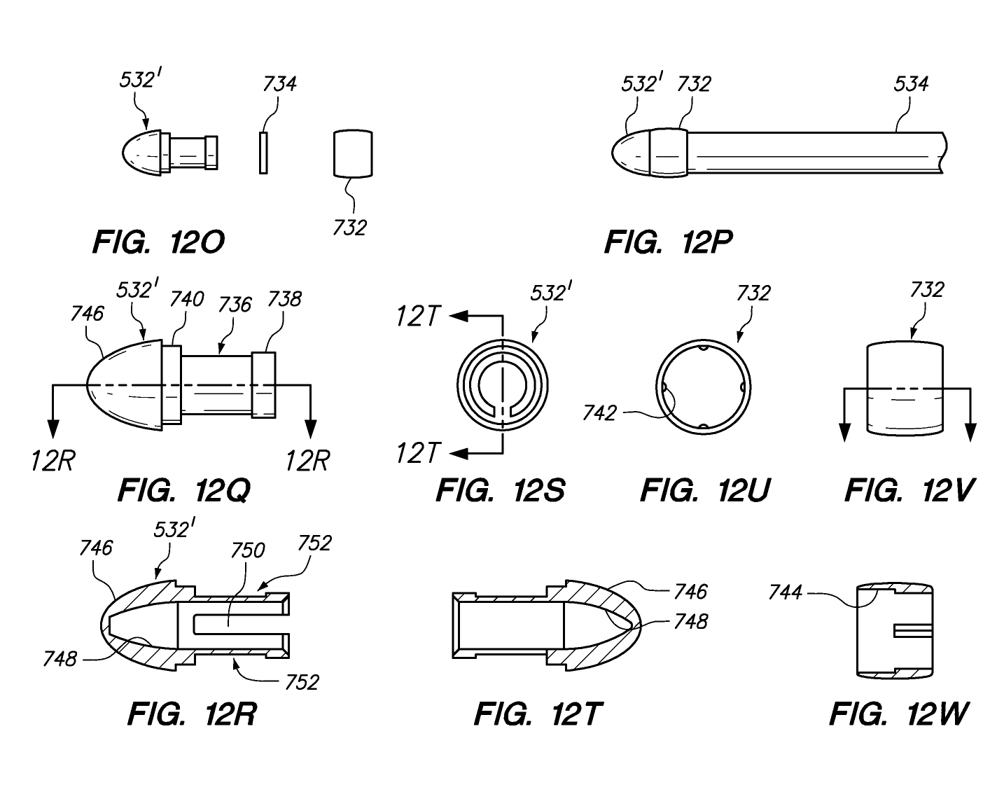

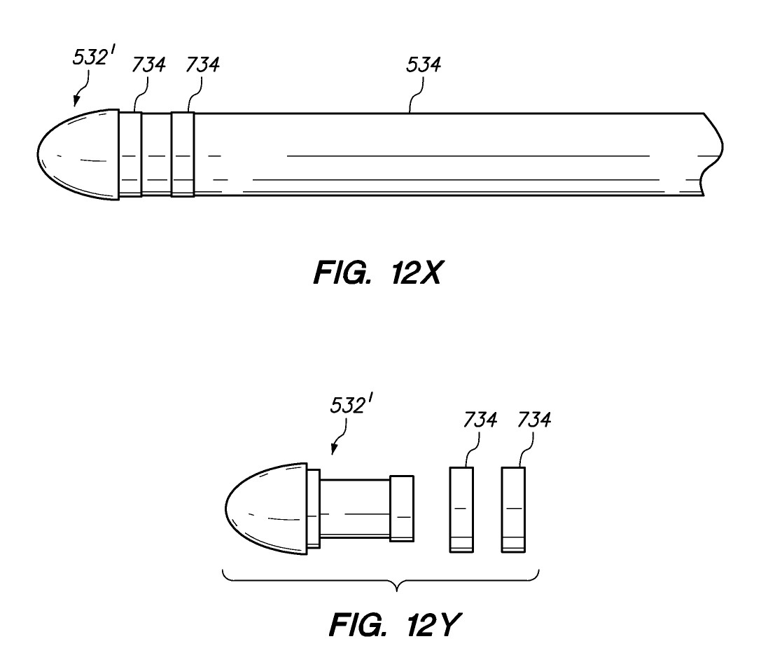

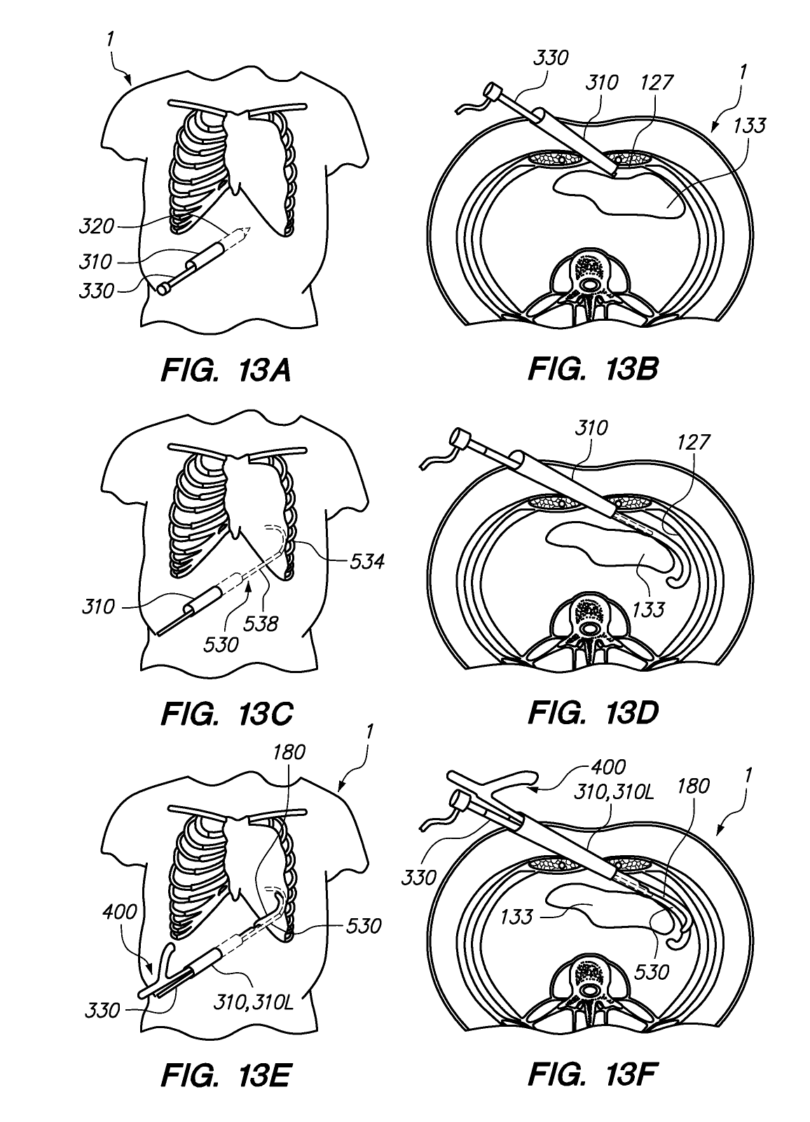

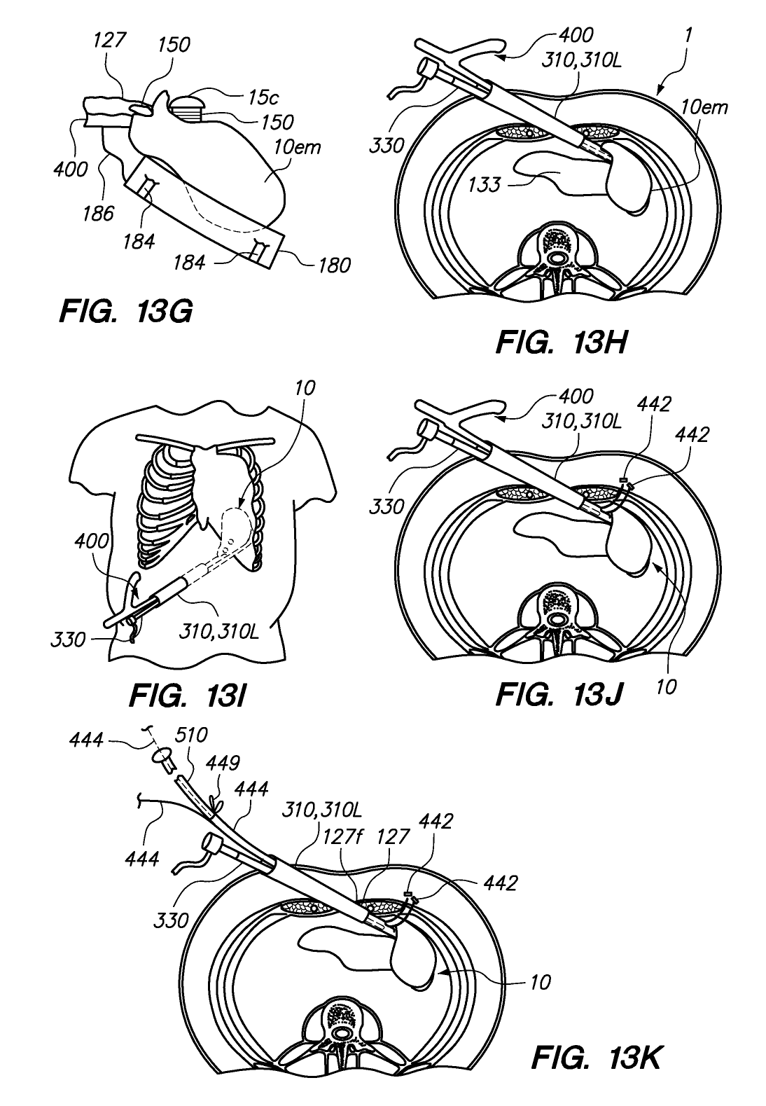

In contrast guide 530 is provided with a blunt atraumatic distal tip 532, as shown in FIGS. 12A and 12B. Guide 530 includes an elongated, flexible tube 534 that has a floppy action at least its distal end portion (excluding distal tip 532) when in an unreinforced configuration, as illustrated in FIG. 12A. Tube 534 may be formed of polyvinyl chloride (PVC) to ensure that the tube is transparent for maximizing visualization via an endoscope 330 inserted therein. Alternatively, polyethylene, polyurethane, PEBAX or MILIFLEX® (thermoplastic elastomer, thermoplastic olefin, Melitek A/s) may be used. Tube 534 typically has a length of about eighteen inches to about twenty-six inches, typically about twenty inches to about twenty-four inches. In one particular example, tube 534 had a length of about 22.5″ and is a single flexible tube, wherein a stylet or rigid endoscope can be slid coaxially within the tube to rigidify it during use, when needed. In another embodiment guide 530 has a length of about 41.75″±0.125″, including measurement of tube 534 and tip 532. In another embodiment, a distal end portion (e.g., distal most length of about three inches to about eight inches, typically about four inches to about seven inches, in one particular embodiment about five and a half inches) may be flexible while the remaining proximal portion is stiff or relatively rigid so that it does not bend under use and therefore does not require the use of a stylet or rigid endoscope 330 to rigidify it. One advantage of this embodiment is that a flexible endoscope 330 can be inserted into guide 530 without the need for a stylet. This arrangement can be advanced without a stylet due to the stiffness of the stiff proximal tube portion of guide 530. Flexible endoscope 330 can be advanced up into the flexible distal portion of guide 530 to provide view along the curved pathway along the diaphragm, for example. FIGS. 12A-12B illustrate an embodiment of guide 530 in which the entire length of tube 534 is flexible and of the same material and construction.

FIGS. 12C-12E illustrate an embodiment of guide 530 in which a distal end portion 534a of tube 534 is flexible, while the proximal end portion 534b of tube 534 is rigid. The tube portions 534a and 534b may be made of the same material composition, but where the hardness of the material composition used to make portion 534b is greater than the hardness of the material composition used to make portion 534a. In one particular embodiment, portion 534b was made from PVC (polyvinylchloride) have a Shore hardness of 100 A, while portion 534a was made from PVC having a Shore hardness of 80 A. The clear tip 532 was also formed of PVC. In another embodiment, portion 534b was made of polycarbonate and tip 532 was injection molded out of polycarbonate. In the embodiment of FIGS. 12C-12E, tip 532 does not have a lumen or opening to allow a guide wire 502 to pass through it but is closed off, thereby preventing inflow of fluids or tissues into the tube 534. Alternatively, this configuration may be provided with a lumen 534 that passes through the distal tip 532 to allow guide 530 to be passed over a guidewire 502. Likewise, embodiments of guide 530 comprising a tube 534 that is flexible over its entire length need not be provided with an opening through tip 532 or at any location of the distal end portion, but may be closed off to prevent fluid inflow, alternative to the embodiment shown in FIGS. 12A-12B. Although not shown, embodiments of guide of the type shown in FIGS. 12C-12E may include one or more radiopaque markers along any locations thereof to facilitate tracking of the guide under fluoroscopy.

The longitudinal sectional view of FIG. 12D illustrates the interconnection of the tube portions 534a and 534b at joint 537. Joint 537 may be a lap joint, a sleeve joint or other known mechanical configuration and/or joined with adhesive, ultrasonic welding, heat welding, etc. Tip 532 is joined to the distal end of tube 534 at joint 539 which may be any of the same types and/or methods of joining described with regard to joint 537. Rigid portion 534b, in one embodiment, had an outside diameter of about 0.5 inches and an inside diameter (formed by the lumen passing therethrough) of about 0.225 to about 0.25 inches.

Optionally, any embodiment of guide 530 described herein may be provided with an extension tube 543 like that illustrated in FIGS. 12C-12E. Extension tube may have a length of about four inches to about ten inches, typically about five to about eight inches and may be rigid or flexible. Extension tube 543 is configured to be maintained outside of the patient's body at all time, but provide an additional length for grasping by the user in instances where nearly all of the tube 534 is inserted into the body. Extension 543 further facilitates introducing a tool or implant/device over the guide 530, particularly when there is not much length of the tube 534 extending out of the patient's body. Optionally, extension tube 543 may be provided to be easily removable, such as by a screw threaded joint with the proximal end of tube 534, for example, to allow installation or removal during use of the guide 530. In instances where extension tube 543 is flexible, it may be bent transversely to the longitudinal axis of the guide 530, as illustrated in phantom lines in FIGS. 12C and 12E. This may be desirable for example for use as an endoscope port, particularly when a flexible endoscope is used. In the particular example shown, extension tube has a length of about six inches, is flexible, and is made of PVC having a Shore hardness of 80 A.

In another embodiment where tube 534 is a single, flexible, transparent tube (FIG. 12L), an outer sleeve 531 (FIG. 12M) is provided that is rigid, thin-walled and fits closely over tube 534 while still allowing tube 534 to freely slide relative to sleeve 531. In this way, sleeve 531 can be slid over tube 534 (whether or not a flexible endoscope 330 has been inserted into the guide 530, see FIG. 12M) to function like the stylet or rigid endoscope described in the embodiment above. Sleeve 531 can be translucent or opaque, but is preferably transparent, and, for example, can be made of PVC or, more preferably, polycarbonate or some other clear material harder than 100 A Shore hardness. Sleeve 531 may be keyed to tube 534 via one or more keys 533 as illustrated in the end view of tube 534 inserted into sleeve 531 shown in FIG. 12N. This keying 533 allows torque to be transferred to guide 530 by the user torquing on sleeve 531, which is useful for steering guide 530 as well as applying other rotational forces for repositioning and/or controlling movements of guide 530.

The outside diameters of tube 534 and tip 532 are typically in the range of about 0.35 inches to about 0.7 inches, typically about 0.5 inches. In one example, tube 534 and tip 532 each have an outside diameter of about 0.4 inches. Tip 532 is blunt and formed of a polymer, such as PVC or acrylic polymer, to ensure that guide 530/tip 532 will not penetrate tissues such as bowel or other internal body structure not intended to be penetrated, and will not cause trauma to any of these tissue or structures. Alternatively, tip 532 can be made with a barium filled polymer and/or stainless steel for radiopacity. Tips may from PVC or acrylic polymer may be provided with a radiopaque band or other type of radiopaque marker that does not obstruct visibility through the portion of the tip 532 designed to be viewed through. Tip 532 and/or tube 534 may optionally be provided with one or more radiopaque markers 536 at any location(s) therealong, to aid fluoroscopic visualization. Rod 538 will typically be made of a material that is visualizable under fluoroscopy and thus will not require a radiopaque marker since it can be visualized without the need for one.

A stiffening rod stylet 538 is provided that is slidable through lumen 540 of tube 534 for the embodiment of FIG. 12A. Stylet 538 may be a solid rod, or alternatively may be tubular and configured to be slid over a coaxial guidewire lumen and/or a guidewire. Accordingly, when the distal end portion of tube 534 does not contain rod 538, it is flexible and floppy and functions similar to the guidewire 502, albeit with a less traumatic tip 532. However, in situations such as when there is too much resistance from fatty tissues or other tissues to allow tube 534 to be pushed along the intended track, rod stylet 538 (or a rigid endoscope 330 or sleeve 531) can be slidably advanced into (or over, e.g., when sleeve 531 is used in the embodiment of FIG. 12M) the distal end portion to increase the stiffness of the distal end portion. Rod/stylet 538 is continuously positional so that the distal tip 539 thereof can be located anywhere along tube 534 with lumen 540. Likewise, sleeve 531 is continuously positionable. Accordingly, the amount of stiffness of the distal end portion of tube 534 is also continuously variably adjustable. In one embodiment rod 538 is formed of aluminum. Alternatively, rod stylet 538 may be formed of any other rigid, biocompatible metal, alloy, polymer and/or ceramic/composite; or the rod 538 can be a rigid endoscope, for example, a glass scope with a steel sleeve for rigidity. Rod 538 can be advanced within tube 534 as described, and this runs no risk of damaging any tissues, since rod 538 is contained entirely within tube 534 and tip 532. Also, the blunt configuration of tip 532 ensures that no tissues such as bowels, diaphragm, etc. will be penetrated or traumatized even when rod 538 has been inserted all the way of the distal tip 532, where guide 530 is in its stiffest configuration. Further since blunt tip 532 is transparent, viewing through it via endoscope 330 is also possible. Accordingly, guide 530 also functions as a blunt introducer, and further provides visualization capabilities.

Tube 534 may optionally be provided with a lumen 542 that runs alongside the main lumen of tube 534 to facilitate delivering guide 530 over a guide wire 502 in an optional alternative procedure, or to deliver anesthetic or other fluids, as described above as well as in examples below. Alternatively, the lumen 542 can allow for an exchange with a guidewire 502. In this embodiment guide 530 would enable placement of a guidewire 502 in a desired location by first enabling the user to place the guide 530 in the desired location. The guidewire 502 would be pre-assembled in the lumen 542, or it could be inserted into the lumen by the user. The guidewire 502 would be pushed out the front (i.e., distal end) of the lumen, while the guide 530 is being retracted from the patient. This exchange would leave the guidewire 502 at the desired location, where it otherwise would not have been possible to place the guidewire without the assistance of the guide 530. The guidewire 502 could then be used to guide the placement of an implant.

FIGS. 12O-12W show an embodiment of guide 530 in which a preferred tip arrangement is provided. Tip 532′ is attached to tube 534 vi a compression fit using band 732. Optionally, adhesive may be used to enhance the connection of tip 532′ and/or to seal the connection. Tip 532 is provided with a blunt exterior curvature 746 that is symmetrical about 360 degrees, i.e., same curvature from the distal tip to the proximal end of the curve, regardless of the location about the circumference of the tip, as the line of curvature extends in the direction of the longitudinal axis of the tip 532′. The exterior curvature 746 is a curved, conical shape. Tip 532′ includes a recessed segment 736 proximal of the curved portion 746. Recessed segment 736 is bordered by shoulder 738 and 740, each of which has an outside diameter greater than the outside diameter of recessed segment 736. This arrangement is configured to receive band 732 over the location of recessed segment 736, where shoulders 738, 740 prevent axial migration of the band 732.

Additionally, a secondary retainer ring 734 may be provided to slide over tube 534 and reside over a portion of segment 736. This secondary retainer ring may also function as a radiopaque marker, and may thus be made of stainless steel or may be a polymer having embedded barium, for example. When secondary retainer ring 734 is used, band 732 is recessed 744 along its inside diameter at a distal portion thereof (see FIGS. 12V and 12W) to receive secondary retainer ring 734 and maintain contact therewith, as ring 734 contacts tube 534 to hold it in compression against segment 736. Likewise, the proximal portion of band 732, which is not internally recessed and therefore has a smaller inside diameter that portion 744, acts as a stop or shoulder against ring 734 and also maintains contact against tube 534, holding it in compressive contact against segment 736. The proximal portion of band 732 may further be provided with protrusions 742 (See FIG. 12U) which extend radially inwardly and further enhance the friction between band 732 and tube 534.

The proximal end portion of tip 532′ includes a slot 750 that separates at least shoulder 738 and segment 736 into at least two portions. Slot 750 is also provided to straddle a secondary lumen that is raised and runs alongside the primary lumen of tube 534. Slot 750 also reinforces the tip-tube joint as the retainer ring forces tip portions 736 and 738 to cinch/grip the raised secondary lumen. Slot 750 allows the portions separated by slot 750 to be flexed toward one another, thereby temporarily reducing the outside diameters of these portions. This facilitates the ease with which the proximal end portion of tip 532′ can be slid into the lumen of tube 534 at the distal end thereof. When tube 534 abuts shoulder 740 (or overlies the same, alternatively) release of compression on the portions of the proximal end portion of tip 532′ allow them to resiliently spring back to the configuration shown in FIGS. 12Q and 12R. Optionally, the proximal portion of tip 738 can be embedded/reinforced with a steel c-spring. Sliding ring 734 and band 732 over the tubing to reside in their respective locations over segment 736, completes the assembly of guide 530, as shown in FIG. 12P.

To improve the optics of the tip 532′, a secondary curvature 748 is provided on the internal surface of the tip. The secondary curvature 748 does not match the curvature 746 on the external surface of tip 532′. In one preferred embodiment, surface 748 is formed to be “duckbill-shaped”. That is, the curvature in one plane (see FIG. 12T, taken in the plane C-C of FIG. 12S, which is a proximal end view of tip 532) is bullet-shaped, which is similar to the curved conical shape of surface 746, but with a sharper angle of curvature, and where the distal tip of the curve is not atraumatic, but much sharper than the external distal tip. In a plane perpendicular to the aforementioned plane, the surface 746 does not come to a point at the internal distal tip, or even an atraumatic soft curve, but rather is flat, e.g., a straight line perpendicular to the longitudinal axis, see the sectional view of FIG. 12R taken along line A-A of the side view of tip 532 in FIG. 12Q. This arrangement reduces artifacts, such as the “halo effect” and other reflections that would otherwise be observed through the tip by an endoscope inserted therein.

FIGS. 12X-12Y shows an alternative connection arrangement for connecting a tip 532, 532′ to a tube 534. In this example, two rings 734 are applied directly over tube 534 to compress it against segment 736. Thus, band 732 is not employed in this arrangement. As in the previous arrangement, adhesive may be optionally be used to enhance the connection of tip 532′ to tube 534 and/or to seal the connection.

Referring now back to FIGS. 11B-11D, in procedures where guidewire 502 is not used, guide 530 can be inserted through cannula 310 in a rigid configuration, where rod 538 has been fully inserted though lumen 540 (or sleeve 531 has been slid over tube 534) to its distal most configuration relative to tube 534, or where a least part of the distal end portion 544 contains a portion of rod 538, or where the rigidity of the proximal tube portion 534b provides the rigidity. In this rigid configuration, guide 530 can be passed and guided through cannula 310, so that tip 532 is directed to the desired location of the diaphragm 116 along which it is desired to follow the curvature of. Upon approaching the diaphragm 116 (one to several centimeters before contacting diaphragm 116) rod 538 (or sleeve 531) is retracted, where it is slid proximally so that rod 538 is no longer contained within (or sleeve 531 is no longer positioned over) at least the distal end portion of tube 534, whereupon the distal end portion of tube 534 regains its floppy characteristic. Further advancement of guide 530 allows the floppy, flexible tube portion (guided by contact of tip 532 against the diaphragm 116) to follow along the curvature of the diaphragm 116, thereby locating guide 530 along a path where it is desired to deliver device 10 along and also locating a target location where device 10 is to be implanted/anchored. In embodiments where a sleeve 531 is used or the proximal end portion of tube is relatively rigid, a flexible endoscope 330 can be inserted into the flexible distal end portion of tube 534 and used to provide visualization as the distal end portion is advance around the diaphragm 116. It is noted that guide 530 can be made steerable by a tip portion thereof including distal tip 532 to be bent and oriented at a predetermined acute angle 546 relative to the longitudinal axis of the main portion of tube 534, as illustrated in FIG. 12E. This type of preset bend can be included in the distal end portion of any of the embodiments of guide 530 described herein and used for steering the guide 530 as it is advanced. In the particular embodiment shown in FIG. 12E, the distal most portion of the tube 534c (including the clear tip 532) is straight and has a length of about 1.75″. The radius of curvature of the bent portion 534d is about 1.5″ in this particular embodiment.

In embodiments where the distal tip portion 534d is angled to the longitudinal axis of guide 530 as a result of presetting a bend in bent portion 534d, as guide 530 is advanced, it is guided generally in the direction in which the tip 532 points. By rotating tube 534, the direction in which tip 532 points can be changed, thereby changing (steering) the direction is which guide 534 is advanced. Tools, instruments and devices can be delivered over guide 530 in the same way that they are delivered over guidewire 502, although the lumens or openings in the tool, instrument or device will, of course, need to be made larger to accommodate the passage of guide 530 therethrough. Alternatively, guide 530 may be provided with a key or rail structure 548 as illustrated in FIGS. 12F-12G, and tools, instruments and devices can be provided with a slotted lumen, slotted rings, or other mating component 549 to be guided over key or rail 548.

In at least one embodiment (such as the one used in FIGS. 11E-11F), guide 530 can be provided with a transparent elastic, inflatable balloon 550 as illustrated in FIG. 12H (deflated). FIG. 12I shows an embodiment of a guide 530 showing balloon 550 in an inflated state. A lumen 552 extending either within tube 534, or externally alongside tube 534 allows pressurized liquid and/or gas to be inputted from a location outside of the patient 1 to inflate balloon 550 when guide 530 has been placed at the target location. An opening 554 is provided in the proximal end of the inflated balloon, to allow tool 400 to be inserted therein. Extending proximally from opening 554 is a conduit 554c with a valve 554v that seals around the tool 400 after it has been inserted, in order to create a seal for inflating the balloon 550. In this embodiment, tool 400 is configured to slidably receive an endoscope 330 therein (FIG. 7J) or is configured with an integral endoscope 330.

Once guide 530 has been placed as desired (which can be confirmed by fluoroscopic visualization), balloon 550 is inflated as shown in the frontal and sectional illustrations of FIGS. 11E-11F. Balloon 550 is a thin-walled, elastic inflatable balloon that is clear when inflated to allow visualization therethrough. When inflated, balloon 550 may take on the general shape (and optionally, volume) of fillable member 10em when filled to at least the initial volume. In one embodiment, balloon 550 is made of silicone. Because of this, balloon 550 can be moved while visualizing under fluoroscopy (the space established by inflating the balloon 550 can be seen under fluoroscopy) to identify an ideal location for placement of the device 10. Upon identifying such location, balloon 550 is immobilized, so that the guidance features of the procedure (i.e., guide 530 and/or guidewire 502) are now located to guide the driving tool 400 to deliver the anchors 440 in locations to establish the ideal location of the device 10 upon implantation thereof.

Next anchor delivery tool 400 is inserted through cannula 310 as illustrated in FIG. 11G. Tool 400 may be guided by guide 530, such as in the case where features 549 are provided on tool 440 (FIG. 7K) or tool 400 may be simply inserted adjacent the guide 530 and guided by cannula 310. In either case, the distal end portion 418 of tool 400 is advanced to balloon 550 and either inserted therein (FIG. 11H) or abutted up against. This positions the distal end 330d (e.g., lens at distal tip) for viewing through the open space provided by the expansion of balloon 550. The expansion of balloon 550 works to push the tissues away from the target site to allow visualization thereof and also to provide ready visualization of the bowel, the fatty tissues, the fascia, etc. Not only does the expanding balloon separate the bowel from the fascia, but it allows the operator to confirm this separation by visualizing the fascia 127 clear of the bowel, with the bowel being visible along another surface of the balloon 550 at a location separated from the fascia 127 at the target site. Visualization through the endoscope confirms that no tissues are being trapped between the tool 400 and the fascia 127 and that no tissues are being trapped between the tool 400 and the costal cartilage 15c.

Next, tool 400 is actuated to attach an implantable device (attached to a distal end portion of tool 400) to the fascia 127 and optionally the costal cartilage 15c in a manner as described above. That is, anchors 440 are driven through the target tissues (e.g., fascia abdominal muscle 127 and costal cartilage 15c) in a manner as described above (e.g., see description of FIGS. 6G-6L). Anchor drivers 410 can be driven directly through the wall of balloon 550 and through the target tissues. This may cause deflation of balloon 550. Upon attachment, the balloon 550 can be separated from guide 530 and left implanted in the patient. Alternatively, balloon 550 may be provided with a “trap door” portion 554 or thickened portion 556 (FIG. 12J), that allows drivers to be driven therethrough, but that upon retraction of drivers 410 back into tool 400, acts to retain the majority of the inflation fluid in balloon 550, either by the trap door 554 closing back up (after having been opened by the driving forces of the drivers 410) or the thickened portion 556 self sealing after removal of the drivers 410.

Further alternatively, an endoscope 330 that is independent of tool 400 may be inserted into balloon 550 to perform the visualization function 434 while the distal end portion 418 of tool 400 is placed outside of balloon 550 between balloon 550 and the tissues (e.g., fascia/abdominal muscle 127 and costal cartilage 15c). Alternatively, tool 400 may be configured to slidably receive endoscope 330 (FIG. 7L) and be provided with a slotted or forked configuration (see slot 439, FIG. 7L) that allows the portion of tool 400 receiving endoscope 330 to be inserted into (or abutted against) balloon 550, while distal end portion 418 is positioned externally of the inflated balloon 550. Both of these alternatives are illustrated in FIG. 12K with endoscope 330 inside inflated balloon 550 and distal end portion 418 external of inflated balloon 550. Note that inflated balloon 550 may be provided with a groove or recess 551 along which distal end portion 418 can be slidably received. This allows portions of the inflated balloon on opposite sides of distal end portion 418 to be able to still expand up against the fascia and costal cartilages to perform the separation function.

Alternative to the procedure described with regard to FIGS. 11C-11H, after performing tasks as described with regard to FIGS. 11A-11B, a guidewire 502 and/or guide 530 may be inserted to be used to guide an anchoring member 190a therealong, as illustrated in FIG. 11I. Although FIGS. 11I-11J show use of a guidewire 502, a guide 530 or combination of guidewire 502 and guide 530 could be alternatively used. Once guidewire 502 and/or guide 530 are placed to follow around the diaphragm 116 as desired, anchoring member 190a is threaded over guidewire 502 (and/or guide 530) and delivered through cannula 310, 310L (optionally through lumen 342) and guided to the target anchoring location as it is pushed distally along the guidewire 502/guide 530. When the target anchoring location has been reached, as illustrated in FIG. 11J, and which can be confirmed using endoscope 330 through cannula 310, 310L (optionally through lumen 344), for example, then anchoring member 190a is anchored to at least one internal body structure (fascia 127f and abdominal muscle 127 in this example) using T-bars 442 and sutures or ribbons 444, or other anchoring features described herein. Tool 400 may be used to perform this anchoring of the anchoring member 190a. Next, device 10 in a compact configuration is guided over guidewire 502 and/or guide 530 in a manner like that described with regard to FIGS. 10A-10B, for example, and docking member 190b of device 10 is docked to anchor member 190a, thereby anchoring device 10 to the at least one internal body structure. Fillable member 10em can then be filled to at least the initial volume (see FIGS. 11K-11L) to check the positioning and orientation of device 10, and then the further procedural steps required to complete the procedure, which have already been described above, can be carried out.

FIGS. 13A-13K illustrate various parts of other embodiments of a procedure for implanting a fillable extra-gastric device 10 in a minimally-invasive manner according to an embodiment of the present invention. After preparing the patient 1 in a manner as described above with regard to the procedure described regarding FIGS. 6A-6P, an incision 223 is made and cannula 310 and trocar 320 are inserted into the incision and advanced in the same manner, and using the same tools as described above with regard to FIGS. 6B-6C, under visualization by endoscope 330. A delivery tract is thus formed as described above.

Once the delivery tract has been opened into the abdominal cavity, as visually confirmed via the endoscope 330, the trocar 320 is removed from the cannula 310, and at FIG. 13B the endoscope 330 is used to visually ensure that the established delivery tract and trajectory defined by tilting the cannula 310 lead into a space between the fascia 127 and the bowel 133. When the user is satisfied that the orientation of the cannula 310 points in a desired direction, endoscope 330 is removed from cannula 310, and guide 530 is inserted through cannula 310 as illustrated in FIG. 13C. As noted above, rod 538 can be slidably adjusted relative to tube 534 to make it relatively rigid as it passes through the fatty tissues and other resistance areas on the path to the diaphragm 116, and the rod 538 is retracted, at least from the distal end portion of tube 534 to allow it to flex and be guided along the curvature of the diaphragm. Once guide 530 has been placed in an intended target location (which may be verified by fluoroscopic visualization, for example) rod 538 is removed and endoscope 330 is inserted into the lumen of tube 534, as illustrated in FIG. 13D. As noted previously, a rigid stylet 330 may alternatively be used to perform the function of the rod 538 as well as the visualization function. As another alternative, a flexible endoscope 330 may be inserted in a flexible tube 534 and a rigid sleeve 531 can be used to advance tube 534 through the fat along the desired trajectory. Further alternatively, a tube 534 that has a relatively rigid proximal end portion may be used to provide column strength for advancing guide 530 along a desired trajectory/delivery tract. At least the distal end portion, and preferably all of tube 534 is made from a clear, transparent material, to facilitate visualization therethrough by endoscope 330. Thus, visualization through the walls of tube 534 can be carried out via endoscope 330 (FIG. 13D). Endoscope 330 has a small enough outside diameter to allow it to be used in guide 530 in the manners described herein. In at least one embodiment an endoscope having a shaft outside diameter of about 5 mm is used. In another embodiment an endoscope having a shaft outside diameter of about 2.7 mm is used. Although the visualization field is not a broad field resembling a large “cave” like that provided by the balloon 550 in FIGS. 11F-11G, the visualization field provided by this technique is more like a long, narrow cave, and this does provide sufficient visualization for the user to verify whether the bowel 133 has been separated from the fascia 127, so that the guide 530 extends therebetween.

When it has been determined that guide 530 has been appropriately placed and separates the abdominal contents (e.g., organs, omentun, bowel 133) from the fascia 127, device 10 can then be inserted. Device 10 is provided in a compact configuration and mounted on an anchor delivery tool 400. In at least one embodiment, device 10 is placed in a compact configuration into pod 180, which is mounted to anchor delivery tool 400 and anchor delivery tool 400 and device 10 (in pod 180) are inserted into the abdominal cavity as illustrated in FIG. 13E. Tool 400 and/or pod 180 may be configured to be guided by guide 530 in a manner as described previously. Alternatively, tool 400 and device 10/pod 180 can be guided by cannula 310, the rigidity of the shaft of tool 400 and visual feedback provided by endoscope 330 in guide 530. FIG. 7M illustrates an embodiment of anchor delivery tool 400 in which device 10 has been inserted into pod 180 in a compacted configuration and pod 180 has been mounted to the distal end portion of tool 400. Pod 180 may be fixed to tool 400 by loops or straps 184 and/or tethered 186 to tool 400. Further alternatively, or additionally, tool 400 may be provided with skewers that are inserted through portions of the attachment tab 150 of device 10 to mount the device 10 and pod 180 to tool 400, which is discussed in more detail below. Attachment tab 150 is mounted over rigid ledge portion 404 and proximal drive portion 408 so that openings 154 in attachment tab 150 are aligned with openings 420 of tool 400, so that anchor drivers 410 can be driven through openings 154 during the anchoring process described below.

Tool 400 and device 10/pod 180 are advanced to position the rigid ledge portion 404 of tool 400 against the costal cartilage 15c, as illustrated in FIG. 13F, in a manner as described above, although with a portion of attachment tab 150 positioned therebetween. Delivery of the tool 400 and pod 180 can be visualized through endoscope 330 located in guide 530. Once tool 400 has been appropriately positioned, such as by contacting the costal cartilage 15c, as noted above, the fillable member 10em is then filled to expand it into its intended configuration, to at least the initial volume. Upon filling fillable member 10em, fillable member 10em escapes from pod 180, as illustrated in FIG. 13G. If pod 180 had been attached by loops or other mechanical fixation 184 to tool 400, these loops or other mechanical fixators break away under hydraulic pressure as fillable member 10em expands and drives pod 180 away from tool 400. In such cases, tether 186 may be provided to maintain attachment of pod 180 to tool 400, allowing pod 180 freedom of movement but also providing the ability to remove pod 180 by removing tool 400. Prior to filling, the position orientation of the device 10 can be adjusted, via manipulation of tool 400, while visualizing device 10/pod 180 under fluoroscopic visualization. After filling some repositioning may be possible while visualizing under fluoroscopy, as illustrated in FIG. 13H. In this regard, one or more radiopaque markers may be provided on the fillable member 10em, attachment member 150 and/or fill tube 12 to aid fluoroscopic visualization, such as has been described in at least one of the above-noted previous applications that were incorporated by reference above. It should also be noted here that the procedure may alternatively be carried out to anchor device 10 to locations other than the costal cartilage 15c and fascia abdominal muscle 127. As just one alternative example, device 10 can be anchored to at least two locations on the fascia abdominal muscle without anchoring to the costal cartilage 15c.

When device 10 has been satisfactorily positioned, such as by visual confirmation under fluoroscopy, device 400 is actuated to drive anchor drivers 410 through openings 420 and 154 and through the tissues to which device 10 is to be anchored, see FIGS. 13I and 13J. Anchors 442 may be T-bars or alternative anchoring features, such as, but not limited to those described below with regard to FIGS. 16A-16D. In one embodiment sutures 444 extending from anchors 442 may be provided with ratchet teeth 444t like described above with regard to FIG. 9G. In this case, once anchor drivers 410 have been retracted back into tool 400, the enlarged portions of the teeth 444t having been compressed or flexed (e.g., in the case of elastically deformed flexures) somewhat while within driver 410, expand back to their original, uncompressed or unflexed dimensions after driver 410 is retracted and thus removed from over teeth 444t. Thus, the enlarged distal portions of the tooth 444t nearest the proximal surface of attachment tab 150 abuts against the proximal surface of attachment tab 150, thereby preventing distal movement of suture 444 relative to attachment tab 150. The portion of suture 444 extending proximally from this tooth (or a few teeth proximally of this location) can then be severed, such as in a manner described above. By repeating this process for each opening 154 in attachment tab 150 and suture 444 passing therethrough, attachment tab 150 and device 10 are thus anchored to the patient as desired and extraneous, proximally extending portions of sutures 444 are severed and removed from the patient. Tool 400 and pod 180 can then also be removed.

Alternatively, each opening 154 in attachment tab 150 may be provided with a ratchet or camming mechanism 154r like that described above with regard to FIG. 9H. In this embodiment, distal end portions of drivers 410 may be preloaded between the arms of the camming mechanisms 154r. Drivers, being smooth metal can then be fired through the openings 154 as described, without the teeth of the cam/ratchet arms 154r being able to bite into the drivers and therefore cam mechanisms 154r do not prevent drivers 410 from being driven distally through openings 154. Upon retraction of the drivers 410, while maintaining the sutures or ribbons 444 in place through the openings 154 (as maintained by the anchoring action of anchors 442), camming mechanisms 154 prevent relative sliding of sutures or ribbons 444 in a distal direction relative to openings 154 by the mechanism described above. The extraneous portions of sutures or ribbons 444 extending proximally of the fixation thereof by camming mechanisms can then be severed and removed from the patient 1. Tool 400 and pod 180 can then also be removed.

In still another embodiment a speed nut 154s such as shown in FIG. 9I may be snapped into or press fit into each of openings 154. Alternatively, speed nuts 154s may be integrally formed in openings 154. The barbs 155 of speed nut may be elastically deformed as driver 410 is fired therethrough. However, upon retraction of driver 410 from opening 154, barbs 155 resiliently return to their undeformed configurations and prevent suture or ribbon 444 from sliding distally through opening 154 relative to attachment tab 50. The extraneous portions of sutures or ribbons 444 extending proximally of the fixation thereof by speed nuts 154s can then be severed and removed from the patient 1. Tool 400 and pod 180 can then also be removed. Endoscope 330 and guide 530 can also be removed, either prior to, concurrently with, or after removal of tool 400 and pod 180.

In still another embodiment tool 400 and pod 180 can be removed from the patient 1 prior to completing the anchoring of attachment tab 150 to the internal body structures. Optionally, endoscope 330 and guide 530 may be removed prior to, or together with tool 400 and pod 180, or endoscope 330 and guide 530 may be left in place, as illustrated in FIG. 13K. Further optionally, guide 530 and endoscope 330 can be removed either before removal of tool 400 and pod 180 (to leave more room to withdraw tool 400 and pod 180) or together with the removal of tool 400 and pod 180, and guide 530 may be reinserted with reinsertion of endoscope into guide 530 or endoscope 330 may be reinserted without guide 530. In any case endoscope 330 may be used to visualize the completion of the anchoring of attachment member 150 to the internal body structures. In the embodiment shown in FIG. 13K, removal of tool 400 and pod 180 leaves sutures or ribbons 444 extending proximally out of cannula 310. In the embodiment of FIG. 13K, a knot pusher tool 510 is next used to push a knot 449 (such as a Roeder knot or slip knot or the like) along each suture 444 from a proximal location on the suture 444 outside of the patient 1 (as illustrated in FIG. 13K) to a location in contact with a proximal surface of attachment tab 150, wherein knot 449, together with anchor 442 and suture 444 maintain the attachment tab 150 in contact with the internal structure it was intended to be anchored to. A knot 449 is so installed along each suture 444, into contact with attachment tab 150. The extraneous portions of sutures 444 extending proximally of the knots 449 against the attachment tab 150 can then be severed and removed from the patient 1, using a cutting feature provided on knot pusher tool 510, endoscopic scissors, or some other cutting tool. Endoscope 330 and guide 530, if still in place in the patient 1, can then also be removed.

Once the anchoring of device 10/attachment tab 150 has been completed according to any of the different embodiments described above, cannula 310 or 310L, can then be removed from the patient access member 80 can be installed in a manner as described above with regard to FIGS. 6O and 6P, fillable member can be filled to the volume desired by the surgeon (typically initial volume until healing of the patient and ingrowth of tissue into tissue ingrowth areas 152 has occurred, although this volume may be greater or less as desired by the surgeon performing the procedure), and the patient 1 can be closed to complete the procedure.

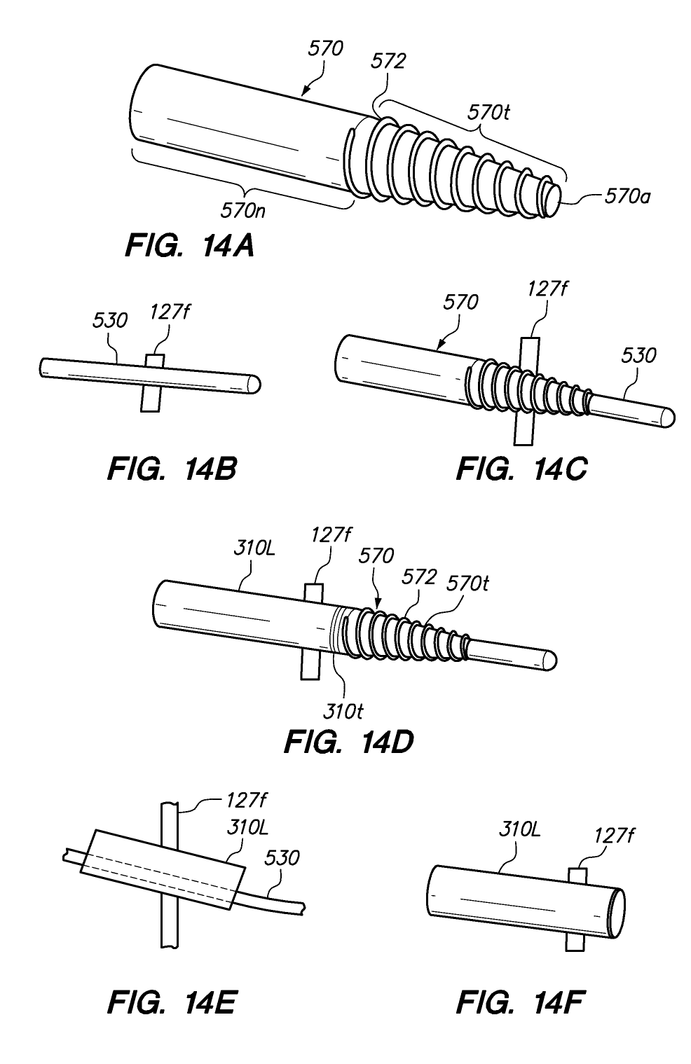

Prior to performing the part of the procedure described above with regard to FIG. 13E, the delivery tract and particularly the opening through the fascia and abdominal wall 127 may be dilated to provide the opening with a larger cross-sectional area to facilitate passage of the tool 400 and device 10 (with or without pod 180) therethrough. FIG. 14A illustrates a dilator 570 that may be used to perform the dilation of the opening through the fascia 127f and/or abdominal muscle 127. Dilator 570 is tapered, with a large threadform 572 along the tapered portion 570t and transitioning to the non-tapered portion 570n. In at least one embodiment the threadform 572 is about 1.5 threads per inch, has a pitch of about 2.67 and wherein the tapered portion has a taper of about eight degrees. In another embodiment, the threadform 572 is about 2.67 threads per inch, has a pitch of about 0.375 and the tapered portion has a taper of about eight degrees. Each of these specification may vary, but the threadform should remain large (e.g. about 1.1 to about 3.3 threads per inch) and the threads should extend sufficiently from the surface of the taper, e.g., about 0.065″ to about 0.125″, typically about 0.080″, but be blunt (rounded) so as to grab the tissues to dive the dilator into the abdominal cavity as the dilator 570 is rotated, without cutting the tissues that the threadform 572 contacts. Dilator 70 has a central annulus or lumen 570a extending therethrough which has a diameter slightly larger than the outside diameter of guide 530. Accordingly, annulus 570a may have a diameter of about 0.5″ or slightly larger. In one particular embodiment dilator 570 has an inside diameter of about 0.505″ formed by annulus or lumen 570a, and an outside diameter of the non-tapered portion is about 0.995″. In another embodiment, the outside diameter of the non-tapered portion is about 1.588″ and the inside diameter is about 0.505″. The distal end of dilator 570, where the tapered portion begins has an outside diameter of slightly greater than the annulus diameter, e.g., about 0.6″ to about 0.7″ and tapers to the cross-sectional dimension of the non-tapered section 570n, which may, for example, have an outside diameter of about 1.0 inches to about 1.7 inches. In another example, the outside diameter of the non-tapered portion 570n was about 1.2 inches. The profile of the threadform 572 can be radiused so that there are no sharp edges on the threadform 572, thereby greatly reducing the risk of trauma. Dilator 570 (including threadform 572) may be made of a relatively rigid, but lubricious polymer, such as DELRIN® (acetal copolymer) or other acetal copolymer, or other suitable biocompatible polymer, such as an injection moldable polycarbonate with or without a radiopaque filler or marker band.

FIGS. 14B-14E schematically illustrate use of dilator 570 to increase the size of the opening in the fascia 127f and/or abdominal muscle 127 so as to make it easier to insert device 10, pod 180 and tool 400 therethrough. FIG. 14B illustrates guide 530 positioned through the fascia abdominal muscle 127 after removal of cannula 310 following the part of the procedure described above with regard to FIG. 13D, for example. Although not shown in the schematic illustration of FIG. 14A for reasons of simplicity of illustration and clarity, endoscope 330 is removed from guide 530 and rod 538 is inserted into guide 530, at least to the extent where rod 538 passes through the opening in the fascia, so as to maintain the orientation of the guide 530 while also providing a low profile arrangement that allows the dilator to be easily passed over the proximal end of guide 530.

Dilator 570, is then slid over the proximal end of guide 530, distal end first and advanced into the opening in the patient. Dilator 570, upon reaching the fascia 127 or even prior thereto, can be rotated (clockwise if threadform 572 is arranged in a right-handed thread or counter clockwise is the threadform 572 is arranged in a left-handed thread to draw the tapered portion through the fat layer (when rotated prior to reaching the fascia 127) and through the fascia abdominal muscle 127. The distal tip of the dilator 570, having the smallest outside dimension, can enter the opening through the fascia 127f by slight pushing (and manipulation such as “wiggling”) on the dilator 570, for example. By further rotating the dilator, the blunt edged threadform 572, threads its way into and through the fascia/abdominal muscle 127 without cutting it, but drawing the tapered portion of the dilator 570 along with it, thus gradually dilating the opening in the fascia 127. Thus, the threadform 572 provides mechanical advantage for enlarging the opening through the fascia/abdominal muscle 127 without cutting, but rather by dilating. Alternatively, the tapered surface of the dilator 570 between the threads could have a texture like a file, which would serve to help break the fascial tissues during dilation. FIG. 14B illustrates dilator 570 being turned to draw the tapered portion 570t through the fascia 127f via the action of the threadform 572 on the fascia 127f.

Continued turning of the dilator 570 continues the drawing of the dilator 570 through the hole in the fascia 127 and/or abdominal muscle 127. A larger cannula 310L (having a cross-sectional opening larger than that of cannula 310 that was previously in place) can be slid over the non-tapered portion of dilator 570 (or pre-mounted thereon) to follow the dilator 570 as it is drawn in through the opening in the fascia, as illustrated in FIG. 14C. Large cannula 310L may have a tapered distal tip 310t that facilitates it following the dilator 570 through the opening in the fascia 127f. In addition, the large cannula 310L may also have threadforms similar to the threadforms 572 on the dilator. Once large cannula 310L has been successfully placed through the opening and across the walls of the fascia and/or abdominal muscle, dilator 570 can be slid out of large cannula 310L and therefore out of the patient 1, leaving the cannula 310L and guide 530 in place, as illustrated in FIG. 14D. Rod 538 may then be removed and endoscope 330 can be reinserted into guide 530, if desired by the surgeon. In at least one other embodiment in which endoscope 330 does not need to be in guide 530 for the portions of the procedure involving inserting, positioning and anchoring the device 10, guide 530 can also be removed along with dilator 570, leaving only the cannula 310L extending through the opening in the fascia, as illustrated in FIG. 14E. At anytime during the dilation procedure, an endoscope can be inserted into the dilator 570 large cannula 310L for viewing through the walls thereof to check whether the introducer has yet passed through the fascial wall. This ability and the features therefore are described in greater detail in co-pending commonly owned, application Ser. No. (application Ser. No. not yet assigned, Attorney's Docket No. EXPL-011, e.g., see FIGS. 11C-11E and the descriptions thereof) filed on even date herewith, and titled “Minimally-Invasive Methods for Implanting Obesity Treatment Devices”.

An alternative embodiment of dilator 570 can be used to install cannula 310L in procedures that use a guidewire 502, but not a guide 530. For example, such a dilator can be used after the part of the procedure described with regard to FIG. 6F or after the part of the procedure described with regard to FIG. 6M. The only difference in the dilator used, is that the annulus 570a can be much smaller as it only needs to be passed over the guidewire 502, not the guide 530. Accordingly, the distal tip of the dilator can also be made to have a much smaller outside diameter.

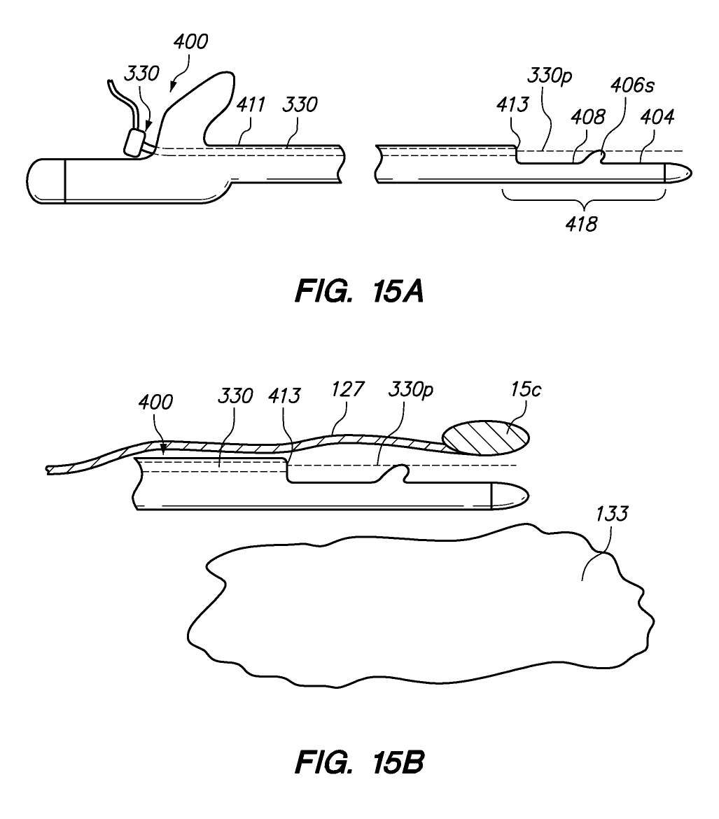

FIG. 15A illustrates another embodiment of anchor delivery tool 400 that can be used in an alternative embodiment of the procedures described above with regard to FIGS. 13A-13K. In this embodiment, anchor delivery tool 400 is provided with a lumen 411 configured to receive endoscope 330 therein. The distal end of lumen 330 may be closed by a clear window 413 that both keeps the lens of the endoscope clean and permits viewing therethrough. The stop 406s of this embodiment is lower profile than stop 406 in other embodiments, to allow visualization therepast via endoscope 330 along visualization pathway 330p. Alternatively, the ledge at the end of the endoscope lumen 413 can function as the rib stop 406s because the user can perform these steps in series and first perform the direct visual check, and then engage the stop 406s on the costal cartilage. Because the costal cartilage can be viewed using the endoscope 330 in this embodiment this also makes it easier to engage stop 406 and portion 404 with the costal cartilage, which also allows the extension height of stop 406s to be reduced relative to stop 406. Alternatively, the stop 406s can be used to “feel” form the costal margin without the aid of visualization, as the user will receive tactile feedback through the tool when the stop 406s bumps up against the costal margin. The tool 400 of FIG. 15A may be provided with a mechanism 549 for tracking over guide 530 or a guidewire 502, but this is typically not necessary as endoscope 330 can visually confirm the pathway that the tool 400 is being inserted along.

When using the embodiment of tool 400 shown in FIG. 15A to carry out an embodiment of a procedure described above with regard to FIGS. 13A-13K, for example, some modifications of the above-described procedures can be made. After successfully locating guide 530 as illustrated in FIG. 13D, endoscope 330 can then be removed from guide 530 while maintaining guide 530 in position. Then, whether cannula 310 is used, or it is replaced by cannula 310L, tool 400 including endoscope 330 can be inserted at FIG. 13E, along guide 530, and endoscope 330 provides visualization of the pathway along which tool 400 is inserted. As the distal end portion 418 of tool 400 approaches the costal cartilage 15c, visualization is still provided through the tool 400 via endoscope 330. At this stage, guide 530 can be removed, if desired by the surgeon, or can be left in place. By viewing through window 413 along pathway 330p, the surgeon is provided with a view by endoscope 330 of the target anchoring site. FIG. 15B illustrates the surgeon's/user's ability to visually confirm that no bowel exists between the distal end portion 418 of tool 400 and the target anchoring sites (in this case, 127 and 15c), prior to contacting the tool 400 thereagainst and actuating the anchor drivers 410 to drive them through the anchoring sites. Upon making this visual confirmation, the operator of tool 400 can then contact stop 406 and portion 404 against the costal cartilage 15c, thereby also drawing portion 408 closer to the fascia 127, whereupon drivers 410 can be driven through these anchoring sites.

If guide 530 has not already been previously removed by the time drivers 410 are driven through the anchoring sites, guide 530 is removed at this time. All of the other parts of the procedure described above with regard to FIGS. 13G to 13K are the same, except that visualization can be performed through tool 400. Alternatively, the guide member 530 may be left in place so that visualization can be performed through it (with an endoscope 330 reinserted) or through tool 400 as described, or both. The attachment of adjustment member 80 and closing of the patient are the same as described above.

FIGS. 16A-16D illustrate alternative anchors 442 that may be used to anchor a device 10 to an internal body structure, such as by attaching an attachment tab 150 thereto. In FIG. 16A, driver 410 or other instrument may be used to drag anchor 442 through the internal body structure, such as, but not limited to fascia 127f and or abdominal wall 127, or costal cartilage 15c. Anchor 442 is provide with a series of ratcheted teeth 442t that allow anchor 442 to be passed distally into and through the body structure, but prevent the anchor 442 from being retracted back out of the internal body structure. Optionally, the material used to make anchor 442t may be made of or coated with a material that encourages tissue ingrowth into it and/or may be bioresorbable. For example, anchor 442 may be composed of a polyester tissue ingrowth material, cut into a shape that has teeth features 442t. Silicone can be incorporated into the teeth features 442t to add stiffness. Another embodiment would have the anchor 442 composed of a suture or ingrowth ribbon that is passed completely through the abdominal wall, allowing the surgeon to suture this anchor 442 into the fascia by accessing it from the exit path though the abdominal wall. FIG. 16B illustrates the anchor 447 fixed in the internal body structure 127, 127f, 15c after retraction of the driver 410 or other tool. A suture or ribbon may extend proximally from anchor 442 to function in any of the same manners described above.

FIG. 16C illustrates another alternative anchor 442. In this case, anchor 442 is formed of NITINOL® (nickel-titanium alloy) or other shape memory nickel-titanium alloy, alloy or metal, or a non-shape memory spring steel. When attachment tab 150 is brought into contact with the internal body structure 127, 127f, 15c to which it is to be anchored, anchor 442 is deployed from a concealed configuration within a lumen 150u within attachment tab 150 to circle through the internal body structure along a pathway defined by the memorized shape of anchor 442 as it is deployed and no longer constrained in a straight configuration by the attachment tab lumen 150u, as shown in FIG. 16C. Thus, anchor 442 passes into the internal body structure at a first location t1, loops around and passes back out of the internal body structure at a second location t2, thereby anchoring the attachment tab 150 to the internal body structure. The sharp distal tip of anchor 442 abuts against an anchor stop 150s provided on the attachment tab 150 to prevent migration of the tip, thereby preventing puncturing of the fillable member 10em as well as trauma to other internal body structures. Optionally, anchor 442 can be coated with a material 442g that encourages tissue ingrowth and/or barbs 442b.

FIGS. 17A-17O schematically illustrate another example of a single opening procedure for percutaneously implanting a fillable paragastric, extra-gastric device 10 according to an embodiment of the present invention. After preparing the patient 1 in a manner as described above with regard to the preparation performed prior to the procedure described regarding FIGS. 6A-6P, an incision 223 is made and a standard, 12 mm diameter, 15 cm length trocar 320/cannula 310 and 10 mm endoscope (shaft has 10 mm outside diameter) 320 are inserted into the incision and advanced in the same manner, and using the same tools as described above with regard to FIGS. 6B-6C, under visualization by endoscope 330. In this embodiment, incision 223 is made at a predetermined distance 223y inferior of the xiphoid process and a predetermined distance 223x to the right of midline of the patient 1, i.e., in the lower right quadrant such as at the linea semilunaris of the patient.

In this particular example, 223y is about 15 cm and 223x is about 6 cm, although these distances may vary. A delivery tract is thus formed as described above, and endoscope 330 is inserted distally to view along the tract up to the bowel or intra-abdominal fat or possibly the location of the stomach 120, as shown in FIG. 17A. The trocar 320 and endoscope 330 are then removed. Guide 530 is next inserted into the tract and a smaller endoscope 330 (e.g., endoscope shaft having about 2 mm to about 5 mm outside diameter) is introduced into guide 530. Guide 530 and endoscope 330 are manipulated in a manner as described above (including the option to use 0.5 liters or less of insufflation as described above) to establish a pathway to and to view the diaphragm 116. Guide 530 is then advanced further, such that the distal portion does not contain endoscope 330 (in the case where a rigid endoscope is used) so that it is floppy and follows around the curvature of the diaphragm 116 as illustrated in FIG. 17B. Endoscope 330 can be used to view the advancement of guide 530 as well as to check the areas surrounding the delivery tract leading to the diaphragm 116.

Next, a local anesthetic, such as Marcaine, or the like can be delivered to the target implantation site through a lumen 535 as illustrated in FIG. 17C. Lumen 535 extends through guide 530 adjacent the main lumen that endoscope 330 is received in. Endoscope 330 is then removed out of guide 530 and cannula 310 is removed from over guide 530 and dilator is used to dilate the opening through the fascia in a manner as described above with regard to FIGS. 14A-14E, to install a large cannula 310L, see FIG. 17D.

Once large cannula 310L has been installed as desired, guidewire 502 is inserted through guide 530, and guide 530 is then removed from the patient 1 while leaving guidewire 502 in place along the delivery tract as shown in FIG. 17E. At this time, or at any time prior to this time, device 10 is rolled or folded into a compact configuration and inserted into pod 180 as illustrated in FIG. 17F. The small endoscope 330 (e.g., shaft having about 2 mm to about 5 mm outside diameter) is inserted into anchoring and delivery tool 400 as shown in FIG. 17G, and pod 180 is mounted on the distal end portion of tool 400. This assembly is then inserted over guidewire 502 and through large cannula 310L as shown in FIG. 17H.

Once pod 180 as been inserted to the vicinity of the target implantation site, guidewire 502 is removed from tool 400 and from the patient 1 as illustrated in FIG. 17I. The position of device 10 relative to the target implantation site can be further adjusted while fillable member 10em is still contained in pod 180 in the compact configuration. Position adjustments can be visually monitored via endoscope 330. When the pod 180 has been positioned and oriented as desired (which may be visually confirmed via endoscope 330), fillable member 10em is next filled (at least partially) with a fluid (saline in this example) via a fluid source 350 connected to fill tube 12, where fillable member 10em is filled with an amount of fluid sufficient to cause fillable member 10em to exit pod 180. The filling of fillable member 10em causes its configuration to change to an increased size and volume that forces it out of pod 180 as illustrated in FIG. 17J and in a manner described in more detail above. As the fillable member 10em fills and thus enlarges, the position and orientation can be further adjusted.

When fillable member has largely exited pod 180, pod 180 is removed from patient 1 by pulling from a tether 183 that extends proximally out of the patient (see FIG. 17J). Alternatively, pod 180 may be connected to tool 400 via tether 183, so that when tool is removed, pod 180 is removed along with it. FIG. 17K illustrates the former alternative, where pod 180 has been removed from the patient 1 and fillable member 10em has been filled to the initial volume. In this configuration, the position/orientation of device 10 may be further adjusted while visualizing under fluoroscopy 360. As noted previously, fillable member 10em and/or fill tube 12 may be provided with one or more radiopaque markers 536 which facilitate observation under fluoroscopy. Adjustments made at this stage are typically fine adjustments. A flexible extension may be optionally provided that extends distally from the distal end of tool 400 and is inserted into a pocket formed in the fillable member 10em (or alternatively, pod 180). This arrangement provides implant 10, with or without pod 180 greater columnar strength, particularly at the distal end portion thereof that extends distally of the rigid portions of tool 400, but at the same time allows some flexibility to allow device 10 to follow around a curved tract such as that provided by the diaphragm 116, for example, or other delivery tract that includes curvature. This arrangement also retains the atraumatic characteristics of the device 10, with or without pod 180, as it/they are being delivered in the compact configurations. An example of this arrangement is shown and described in greater detail below. When device 10 has been positioned and oriented as desired, actuator 416 is next used to actuate deployment of needles 410 through the internal body structures (in this case, fascia 127f and abdominal muscle 127 and into the subcutaneous fat 131) to deploy T-bars 442 in any of the manners described above. In this particular example, needles automatically retract after deploying to their furthest extent, and, as they retract, T-bars 442 are automatically ejected therefrom and implanted in a manner as described above with regard to FIGS. 8C-8F, for example. The sutures 444 extending from anchors 442 are next cinched, using sliding knots, speed nuts, or other fixation features, to secure device 10 against the fascia 127f and the excess suture extending proximally from the cinched location in each suture is severed and removed from the patient.

Tool 400 is then decoupled from device 10 and removed from the patient 1, the results of which are shown in FIG. 17N. Large cannula 310L can also be removed from the patient at this time, the results of which are also shown in FIG. 17N.

Adjustment member 80 is next connected to fill tube/inflation tubing 12 and implanted through the same opening 223 used to perform the implantation of fillable member 10em. Fixation and implantation of adjustment member 80 may be according to any of the variations described above. Further alternative adjustment members 80 that may be employed are described in co-pending applications having been incorporated by reference above.

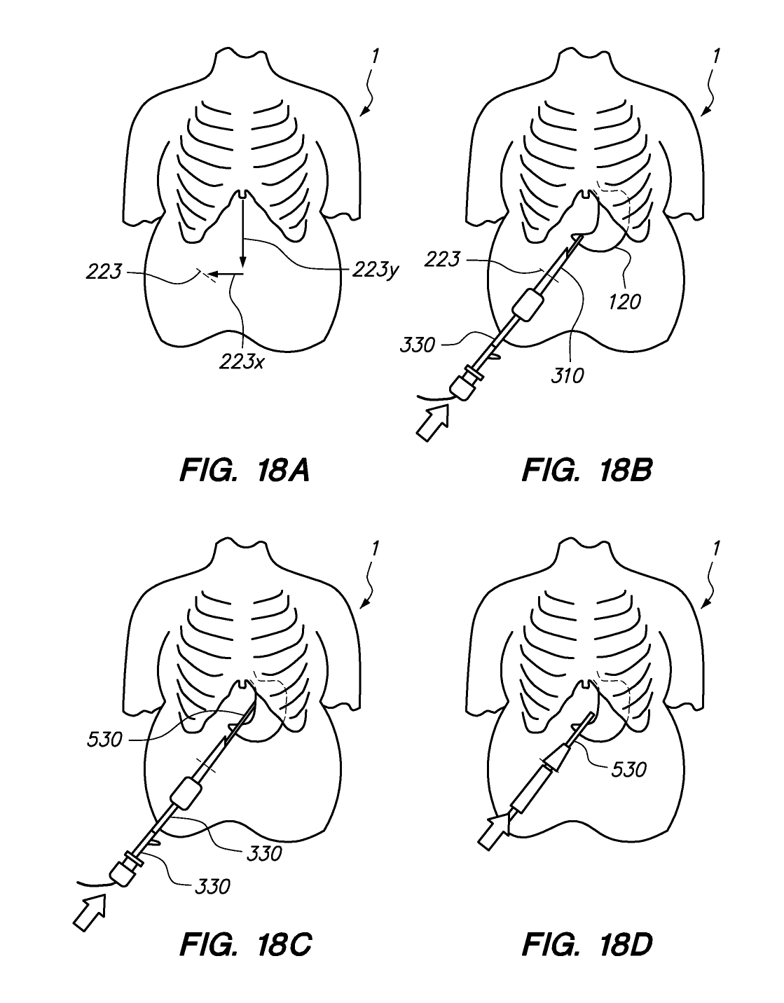

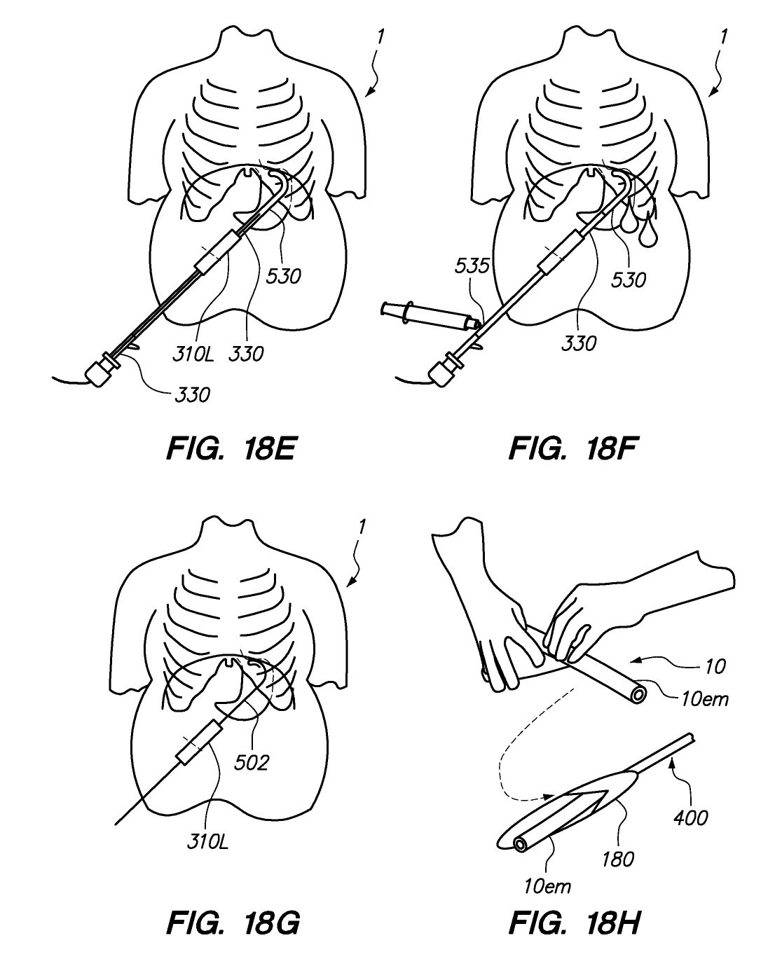

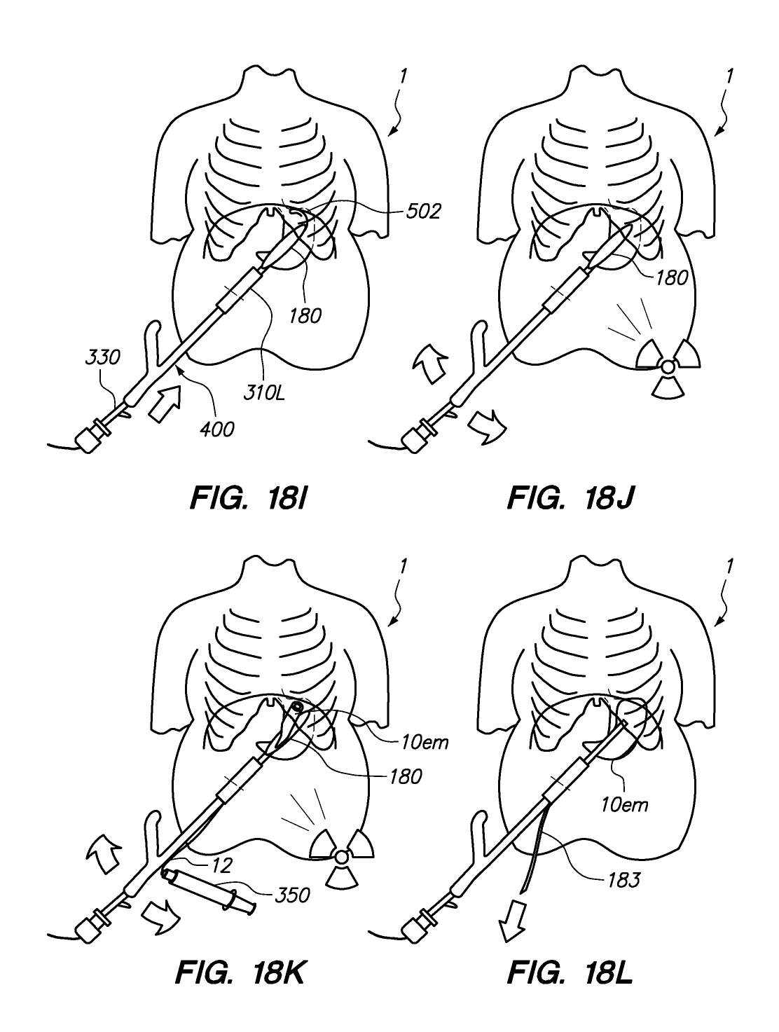

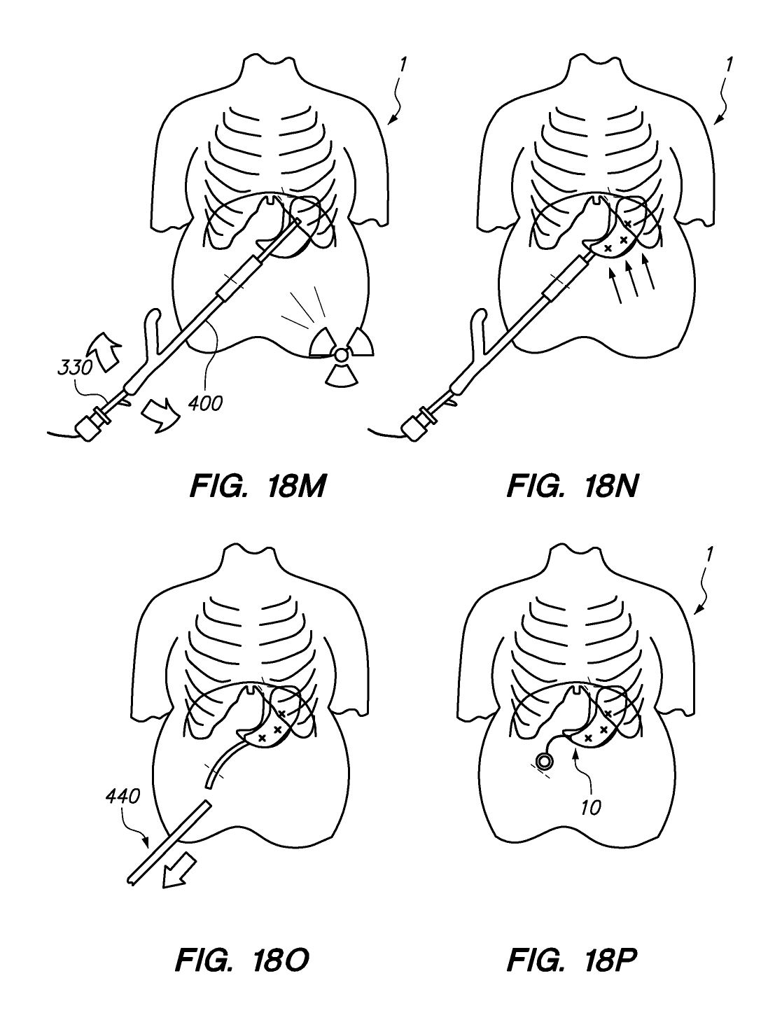

FIGS. 18A-18P schematically illustrate another example of a single incision procedure for percutaneously implanting a fillable extra-gastric, paragastric device 10 according to an embodiment of the present invention. After preparing the patient 1 in a manner as described above with regard to preparation of the patient for the procedure described regarding FIGS. 6A-6P, an incision 223 is made and a standard, 12 mm diameter, 15 cm length trocar 320/cannula 310 and 10 mm endoscope (shaft has 10 mm outside diameter) 320 are inserted into the incision and advanced in the same manner, and using the same tools as described above with regard to FIGS. 6B-6C, under visualization by endoscope 330. In this embodiment, incision 223 is made at a predetermined distance 223y inferior of the xiphoid process and a predetermined distance 223x to the right of midline of the patient 1, see FIG. 18A. In this particular example, 223y is about 15 cm and 223x is about 6 cm, although these distances may vary. A delivery tract is thus formed as described above, and endoscope 330 is inserted distally to view along the tract up to the location of the intra-abominal fat or possibly the stomach 120, as shown in FIG. 18B. The trocar 320 and endoscope 330 are then removed. Guide 530 is next inserted into the tract and a smaller endoscope 330 (e.g., endoscope shaft having about 2 mm to about 5 mm outside diameter) is introduced into guide 530. Guide 530 and endoscope 330 are manipulated in a manner as described above to establish a pathway into a space between the fascia and the bowel, see FIG. 18C.

The cannula 310 and smaller endoscope 330 are then removed while leaving the guide 530 in place as shown in FIG. 18D. Dilator 570 is next screwed through opening 223 and the opening through the fascia to enlarge the opening through the fascia/abdominal muscle 127f/127, to install a large cannula 310L, see FIG. 18D. Once large cannula 310L is installed through the enlarged opening in the fascia, dilator is removed, and guide 530 with the smaller endoscope received therein, is inserted through the large cannula 310L, see FIG. 18E. Guide 530 is rigidified by endoscope 330 which acts as a stylet as the guide 530 and endoscope 330 are advanced to establish the delivery tract to the diaphragm, between the fascia and bowel, and to view the diaphragm 116. Guide 530 is then advanced further, such that the distal portion does not contain endoscope 330 so that it is floppy and follows around the curvature of the diaphragm 116 as illustrated in FIG. 18F. Endoscope 330 can be used to view the advancement of guide 530 as well as to check the areas surrounding the delivery tract leading to the diaphragm 116.

Next, a local anesthetic, such as Marcaine, or the like can be delivered to the target implantation site through a lumen 535 in the guide 530 as illustrated in FIG. 18F. Lumen 535 extends through guide 530 adjacent the main lumen that endoscope 330 is received in.

At this time a guidewire 502 is inserted through lumen 535 to extend distally of the distal end opening of lumen 535, and then guide 530 and endoscope 330 are removed from the patient 1 while leaving the guidewire in place, as illustrated in FIG. 18G. At this time, or at any time prior to this time, device 10 is rolled or folded into a compact configuration and inserted into pod 180 and attached to tool 400 as illustrated in FIG. 18H. The small endoscope 330 (e.g., shaft having about 2 mm to about 5 mm outside diameter) is inserted into anchoring and delivery tool 400 and the tool 400 including the scope 330 and pod 180 are inserted through the cannula 410L after threading the proximal end of guidewire 502 through tool 400 and/or pod 180 as shown in FIG. 18I.

Once pod 180 has been inserted to the vicinity of the target implantation site, guidewire 502 is removed from tool 400 and from the patient 1 as illustrated in FIG. 18J, and the position of pod 180 can be adjusted, if necessary, while monitoring it under fluoroscopy. Thus, the position of device 10 relative to the target implantation site can be further adjusted while fillable member 10em is still contained in pod 180 in the compact configuration. Position adjustments may also be visually monitored via endoscope 330 and/or fluoroscopic visualization. When the pod 180 has been positioned and oriented as desired (which may be visually confirmed via endoscope 330 and/or fluoroscopic visualization), fillable member 10em is next filled (at least partially) with a fluid (saline in this example) via a fluid source 350 connected to fill tube 12, where fillable member 10em is filled with an amount of fluid sufficient to cause fillable member 10em to exit pod 180. The filling of fillable member 10em causes its configuration to change to an increased size and volume that forces it out of pod 180 as illustrated in FIG. 18K and in a manner described in more detail above. As the fillable member 10em fills and thus enlarges, the position and orientation can be further adjusted. This filling process and further adjustments can also be visually monitored under fluoroscopy.

When fillable member has largely exited pod 180, pod 180 is removed from patient 1 by pulling from a tether 183 that extends proximally out of the patient (see FIG. 18L). Further adjustments of the position of expandable member 10em can be made at this time if needed, by manipulating tool 400 while monitoring movements of expandable member 10em under fluoroscopic visualization and/or visualization provided by endoscope 330 as illustrated in FIG. 18M. Once device 10, particularly expandable member 10em has been positioned in the desired position for implantation, endoscope 330 provides visualization to confirm that the attachment area is clear of bowel. For example, for attachment to the fascia 127f, visualization through endoscope 330 can confirm that no bowel exists between the attachment tab 150 and the fascia 127f. Once this has been visually confirmed, tool 400 is actuated to deploy the drivers 410, thereby deploying anchors (e.g., T-bars) 442 through the attachment structure, the drivers are retracted to leave the anchors in place, and sutures are cinched up against the attachment tab and attachment site to anchor device 10 in place via anchors 442, sutures 444 and knots or other fixation mechanism, with the attachment tab 150 and internal body structure attachment site sandwiched between the anchors 442 and knots or other fixation mechanism via sutures 444, FIG. 18N.

Tool 440 is then decoupled from device 10/attachment tab 150, and tool 400 is removed from the patient as illustrated in FIG. 18O. Fill tube 12 extends out of opening 223. At FIG. 18P, fill tube 12 is cut to the appropriate length to join adjustment member 80 thereto and to reduce any excessive length of fill tube 12 that might otherwise exist. After securing adjustment member 80 to the fascia 127f/abdominal wall 127 to both anchor it as well as to close the opening through the fascia 127f, any adjustment of the volume of expandable member can be performed as needed, and then the patient can be closed, including closing of opening 223 to complete the procedure.

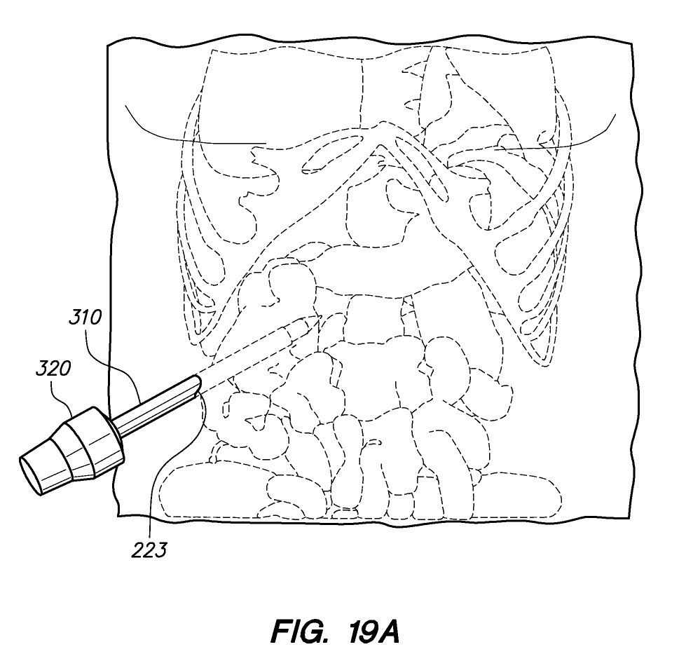

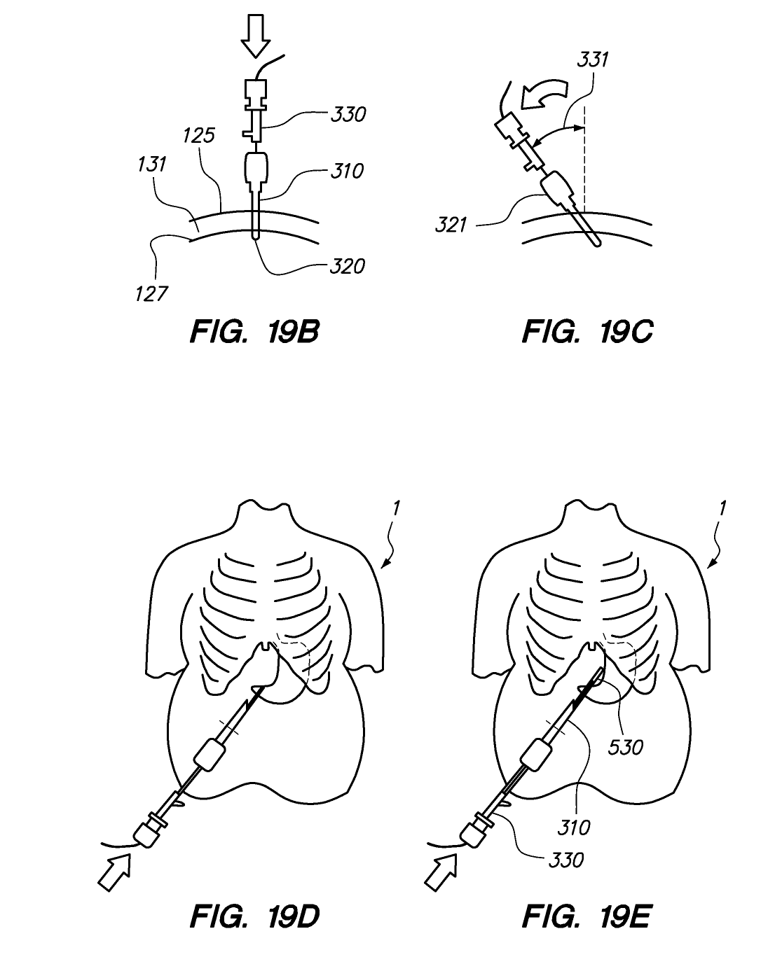

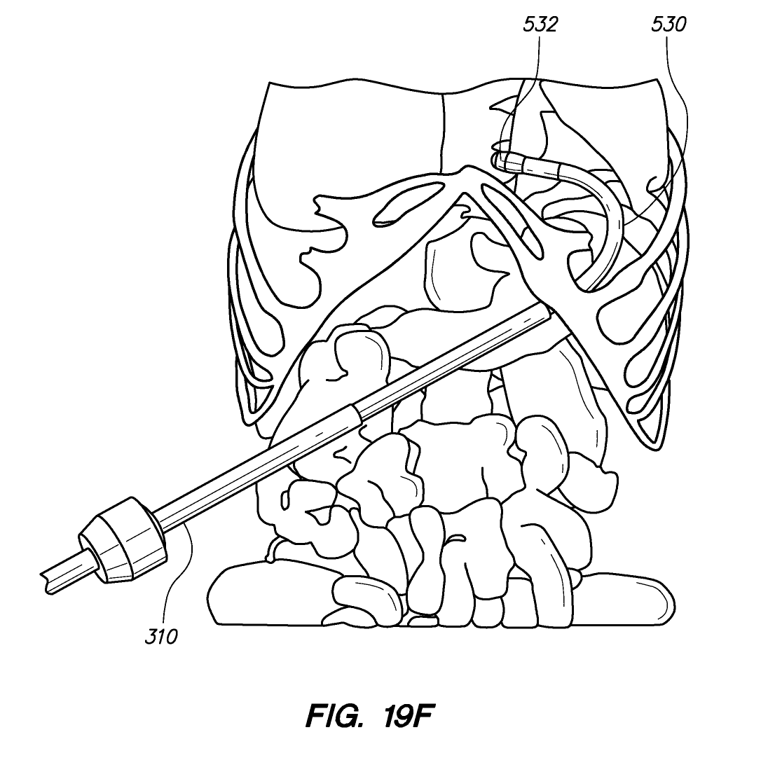

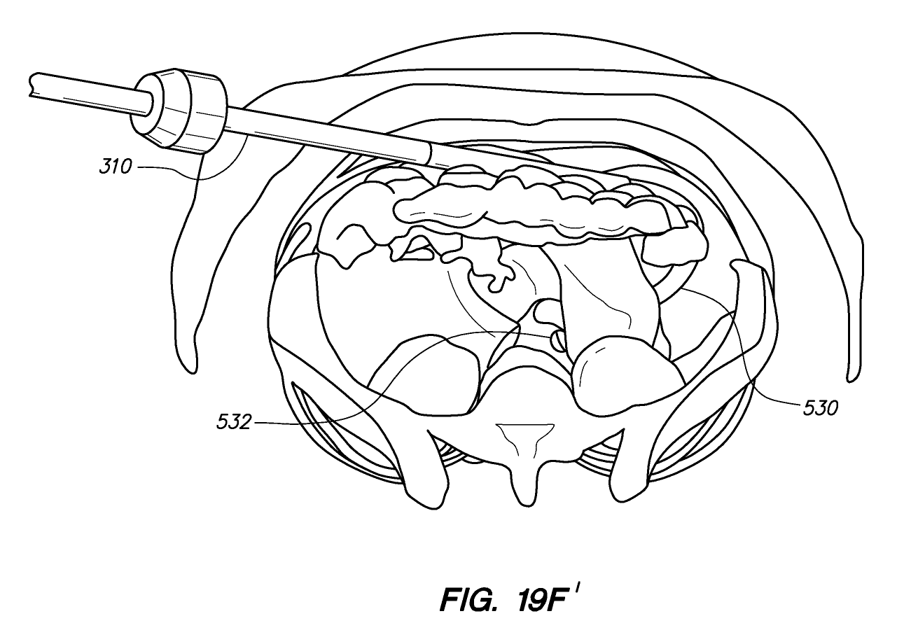

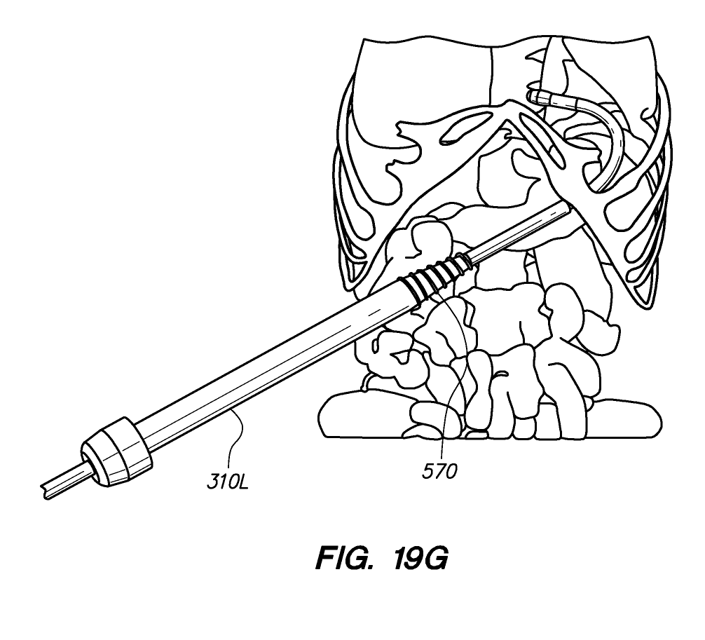

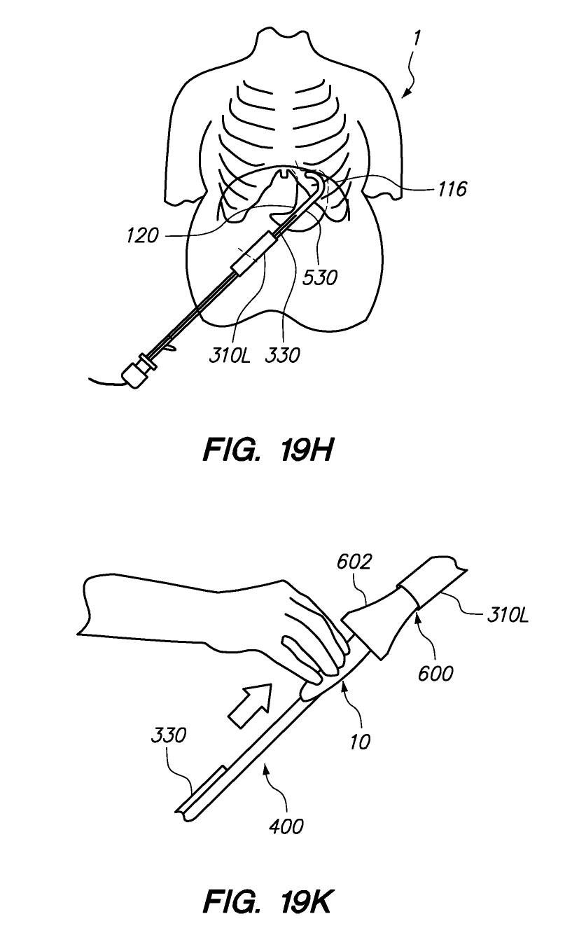

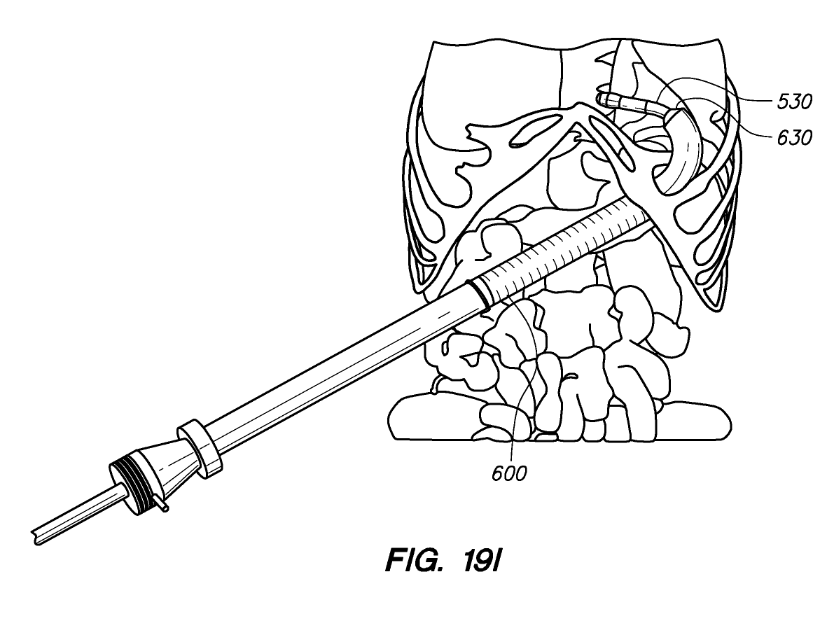

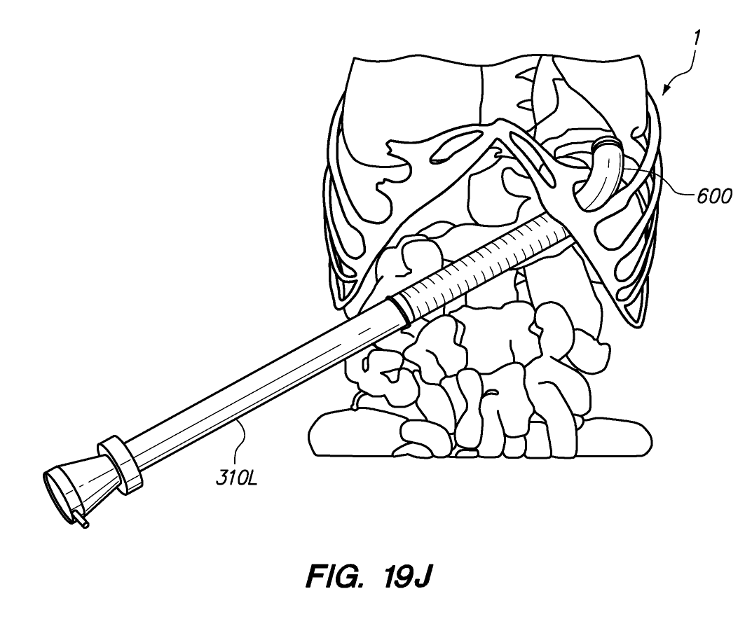

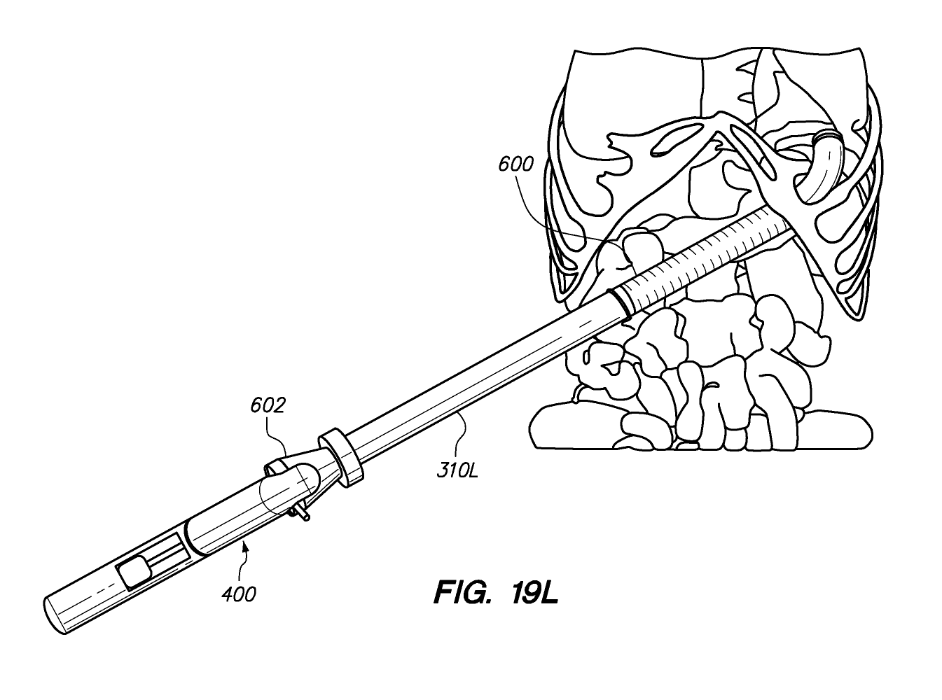

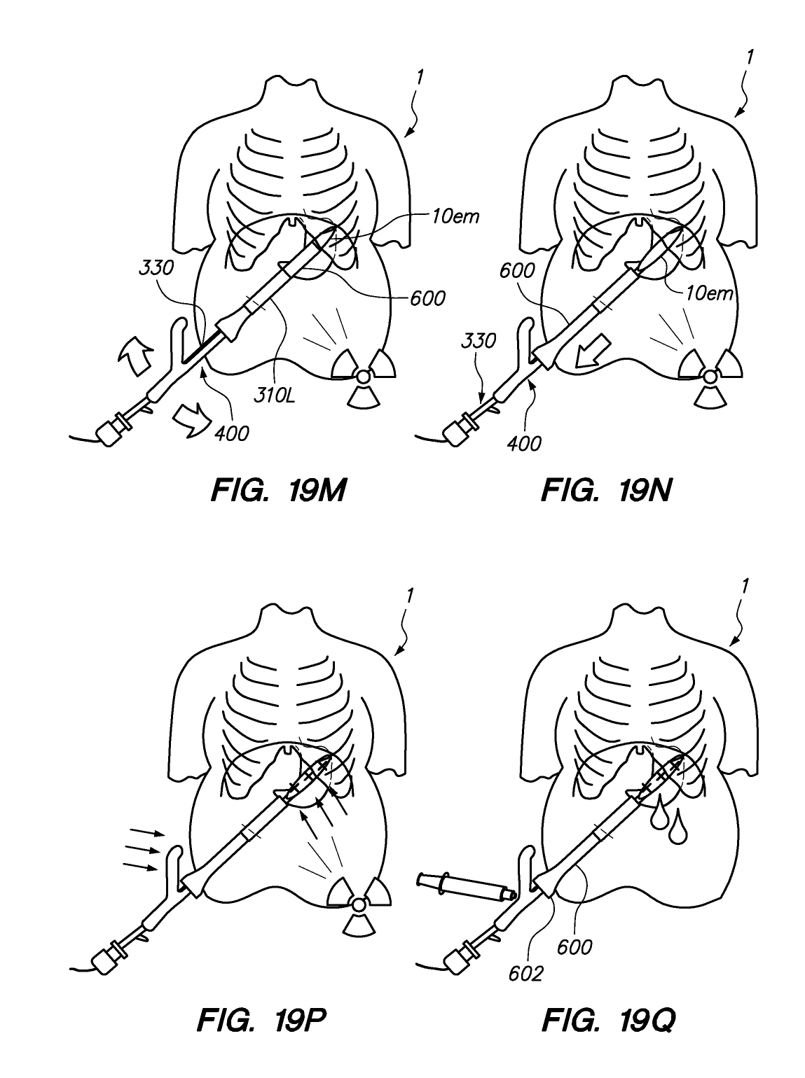

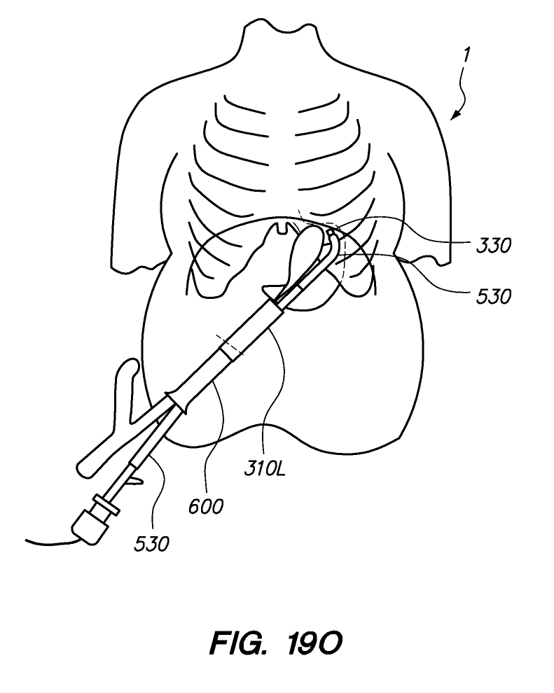

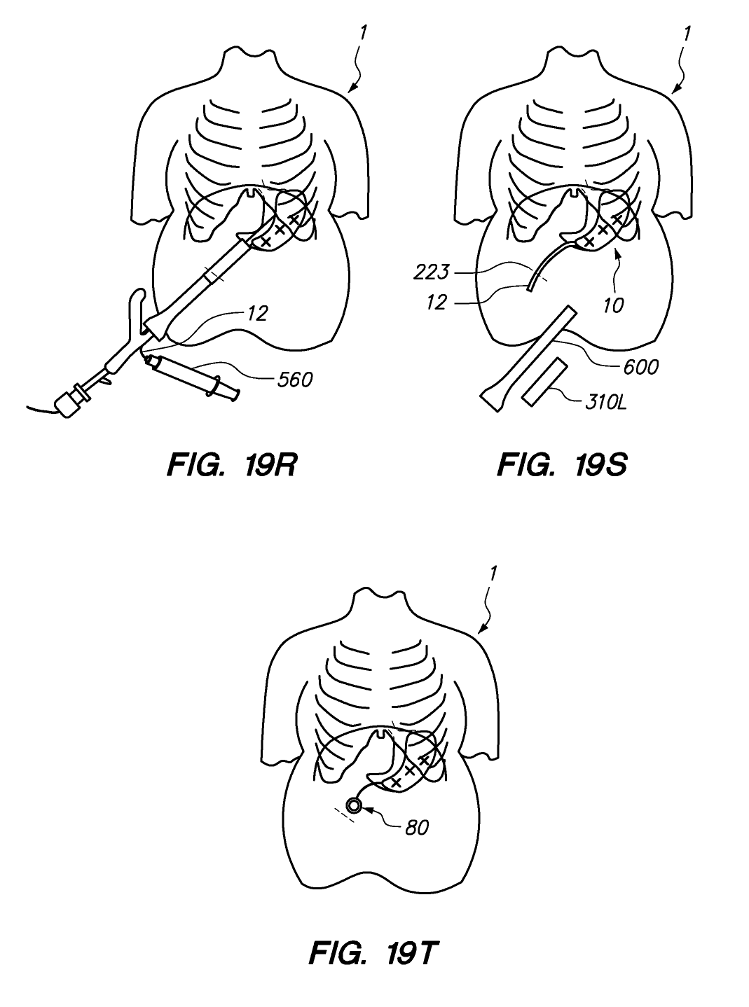

FIGS. 19A-19T illustrate another example and variations thereof of a single incision procedure for percutaneously implanting a fillable paragastric, extra-gastric device 10 according to an embodiment of the present invention. After preparing the patient 1 in a manner as described above with regard to preparing the patient 1 for the procedure described regarding FIGS. 6A-6P, an incision 223 is made and a trocar/cannula 320/310 (e.g., a standard 12 mm diameter, 15 cm length trocar/cannula) and 10 mm endoscope (shaft has 10 mm outside diameter) 330 are inserted into the incision and advanced in the same manner, and using the same tools as described above with regard to FIGS. 6B-6C, under visualization by endoscope 330. In this embodiment, incision 223 is made at a predetermined distance 223y inferior of the xiphoid process and a predetermined distance 223x to the right of midline of the patient 1, see FIG. 19A, like the schematic illustration of FIG. 18A. In this particular example, 223y is about 15 cm and 223x is about 6 cm, although these distances may vary. Initially, the trocar 320, cannula 310 and endoscope 330 are inserted into incision 223 at a substantially perpendicular orientation to the surface of the skin 125, as schematically illustrated in FIG. 19B. Once the sharpened tip of the trocar 320 has passed through the fascia 127f/abdominal muscle 127 and it and the distal tip of the cannula 310 have entered the abdominal cavity, the trajectory of the cannula 310, trocar 320 and endoscope 330 is flattened relative to the skin of the patient surrounding the incision 223, as schematically illustrated in FIG. 19C (and which orientation is also illustrated at FIG. 19A) to form an angle 331 relative to the original, perpendicular orientation of greater than about 60 degrees, typically greater than about 80 degrees, and, in some embodiments, 90 degrees or more. A delivery tract is thus formed as described above, and endoscope 330 is inserted distally to view along the tract up to the location of the intra-abdominal fat or possibly as far as the location of the stomach 120, as shown in FIG. 19D. The trocar 320 and endoscope 330 are then removed. Guide 530 is next inserted into the tract and a smaller endoscope 330 (e.g., endoscope shaft having about 2 mm to about 5 mm outside diameter) is introduced into guide 530. Guide 530 and endoscope 330 are manipulated in a manner as described above to establish a pathway into a space between the fascia and the bowel, see FIG. 19E. If a flexible endoscope 330 is used alternatively to the rigid endoscope 330 shown in FIG. 19D, then viewing can be extended up to and along the diaphragm 116, for example, as illustrated in FIG. 19F. FIG. 19F′ illustrates a sectional view, where it can be readily observed that the tip 532 of the guide 530 also traverses around the stomach and dives down into the abdominal cavity as it is guided by the curvature of the diaphragm.

The cannula 310 and smaller endoscope 330 are then removed while leaving the guide 530 in place. Dilator 570 is next screwed through opening 223 and the opening through the fascia to enlarge the opening through the fascia abdominal muscle 127f/127, to install a large cannula 310L, see FIG. 19G. Once large cannula 310L is installed through the enlarged opening in the fascia, dilator 570 is removed, the smaller endoscope 330 can be reinserted into guide 530, which now extends through the large cannula 310L, see FIG. 19H. Guide 530 is rigidified by endoscope 330 (when a rigid endoscope 330 is used) which acts as a stylet as the guide 530 and endoscope 330 are advanced to establish the delivery tract to the diaphragm, between the fascia and bowel, and to view the diaphragm 116. Guide 530 is then advanced further, such that the distal portion does not contain endoscope 330 (when a rigid endoscope is used) so that it is flexible and follows around the curvature of the diaphragm 116 as illustrated in FIG. 19H. When endoscope 330 is flexible, it can be inserted into the distal portion of guide 530 and follow with it along the bending trajectory that follows along the curvature of the diaphragm. Endoscope 330 can be used to view the advancement of guide 530 as well as to check the areas surrounding the delivery tract leading to the diaphragm 116. As noted, a flexible endoscope 330 may alternatively be inserted so that it remains within the flexible distal end portion of guide 530 as it is advanced along the diaphragm, so that this travel can be visualized via endoscope 330. This alternative is described in further detail below. Otherwise, when a rigid endoscope 330 is used as in FIG. 19H, the flexible distal end portion of guide 530 can be tracked under fluoroscopy when one or more radiopaque markers are included on the flexible distal end portion of guide 530.

Endoscope 330 is next removed, and a conduit 600 and obturator 630 are inserted into the abdominal cavity, being guided over guide 530 as illustrated in FIG. 19I. Once the distal end of the conduit 600 has been advanced to a position adjacent the diaphragm 116 (when a rigid conduit 600 is used), or adjacent to the target implantation site after following around the curvature of the diaphragm 116 when a flexible conduit 600 as used as illustrated in FIG. 19I, guide 530 and obturator 630 are removed, leaving conduit 600 in position for guiding delivery of device 10, as illustrated in FIG. 19J. Alternative to use of a rigid conduit 600, a flexible conduit 600 and flexible obturator are preferably use, as shown in FIGS. 19I-19J. At least the distal end portion of each of conduit 600 and obturator 630 is flexible, although at least conduit 600 can be flexible over a majority of its length, from the distal end toward the proximal end, and the entire length may be flexible. In a preferred embodiment of conduit 600, the proximal fourteen inches are rigid and the distal 10.7 inches are flexible. The flexible distal end portions are configured to follow the flexible distal end portion of the guide 530 so that the distal end portion of the conduit can be delivered along the diaphragm 116 close to or flush with (or even extending slightly distally of) the distal end of guide 530, as described in further detail below.

The delivery and attachment tool 400 having received the smaller endoscope (e.g., 5 mm or 2.7 mm endoscope) therein and having had device 10 mounted thereon, where device 10 is in a compact configuration, is next operated to insert the device 10 and tool 400 into the conduit 600 as illustrated in FIG. 19K.

FIG. 7N illustrates an embodiment of anchor delivery tool 400 in which distal end portion 418 been inserted between at least one layer of attachment tab 150 and fillable member 10em to mount device 10 to tool 400. Further alternatively, or additionally, tool 400 may be provided with elongated supports that are inserted through portions of the attachment tab 150 of device 10 to mount the device 10. Attachment tab 150 is mounted over distal end portion 418 so that openings 154 (three of which exist in the examples of FIGS. 7N and 7O) are aligned with openings 420 of tool 400, so that anchor drivers 410 can be driven through openings 154 during the anchoring process.

FIGS. 7N and 7O illustrate fillable member 10em having been filled to at least the initial volume. However, fillable member 10em, whether encased in a pod 180 or mounted directly to tool 400 without a pod 180 is maintained in a compact configuration as described above, when is it delivered through conduit 600 using tool 400. Only after fillable member 10em has been delivered out of the distal end of conduit 600 is it filled. FIG. 7O illustrates the arrangement of an attachment tab 150 relative to fillable member 10em according to one embodiment of a device 10. Filling fill tube 12 approaches the inferior end portion of fillable member 10em tangentially where it connects thereto. Thus, fill tube 12 is nearly perpendicular to the longitudinal axis LS of the small end portion of fillable member 10em where it connects therewith, forming an angle with the axis LS of about 60 degrees to about 90 degrees. One or more stress reinforcement features 12a may be provided where fill tube 12 connects to expandable member 10em.

Alternatively, tool 400 may be provided without a lumen for receiving endoscope 330 which reduces its cross-sectional size, as illustrated in FIG. 19L. In this case, after the device 10 is delivered out of the distal end of conduit 600, endoscope 330 (either alone, or inserted within guide 530) can be reinserted into conduit 600 and extended distally therefrom to provide visualization of the subsequent implantation procedures. Note that a pod 180 is not used to enclose the fillable member 10em in this embodiment but the fillable member 10em is compacted into a compact configuration by providing it in an empty configuration and rolling or folding it up. Optionally, a vacuum can be drawn within the fillable member 10em to help it maintain its compact configuration while it is delivered to the target site. The proximal end portion of conduit 600 may be provided with a flared or funnel shape 602 to provide a relatively larger inside diameter at the proximal opening of the conduit that tapers down to the inside diameter of the annulus of the generally cylindrical configuration of the main body of the conduit 600. Alternatively, funnel portion 602 may be a separate component that can be joined with the conduit 600, such as by screwing these components together, or other mechanical and/or chemical joinder. At least the inner surface of conduit 600 and funnel portion 602 may be provided with a lubricious surface, such as polytetrafluoroethylene, or polyurethane and polyvinylpyrrolidone, or other generally lubricious, biocompatible material. Additionally or alternatively, a lubricant may be applied to the inner surface of the conduit 600 and funnel portion 602, e.g. sodium stearate, K-Y Jelly, Surgilube, and/or other biocompatible lubricant. Further additionally or alternatively, the device 10 in the compact configuration may be coated with one or more of the same lubricants described above.

At FIG. 19K device 10 is advanced into the abdominal cavity by advancing tool 400 relative to conduit 600 until the distal end portion of the compacted fillable member 10em is located at or extends distally of the distal end of conduit 600, as shown in FIG. 19M. This location of the device 10 can be determined by one or more of monitoring the amount of the tool 400 that remains proximal of the proximal end of conduit 600, as the length of the tool 400 with device 10 mounted thereon relative to the length of conduit 600 may be known or predetermined; visual monitoring via endoscope 330; and/or visual monitoring by fluoroscopy. At this time, the position of the fillable member 10em relative to the anatomy can also be adjusted, if needed, using tool 400 and/or conduit 600 to adjust the position of the fillable member 10em while monitoring movements of the fillable member using fluoroscopic visualization. Further repositioning of the device 10/fillable member 10em can be performed at this time as well, using tool 400 under fluoroscopic guidance.

Conduit 600 is next retracted relative to tool 400 to fully expose the compacted expandable member 10em as shown in FIG. 19N. This action can also be visually monitored under fluoroscopy. FIG. 19O shows an alternative embodiment, where device 400 does not receive endoscope 330, and where, after expandable member 10em has been exposed out of the distal end of conduit 600, guide 530 having received endoscope 330 are inserted through conduit 600 to provide visualization of the device 10em at the target site. Endoscopic visualization via endoscope 330 is used to confirm that the attachment location is clear of bowel, e.g., that the tool 400 and attachment tab 150 are positioned so that a clear pathway to the attachment site exists, such that no bowel, excessive fat or other obstruction exists between the attachment tab and the attachment location, such as the abdominal wall, costal cartilage, or other internal body structure to which device 10 is to be attached. When a clear pathway has been confirmed, the operator manipulates tool 400 via the handles to leverage the attachment tab 150 against the attachment site so that the attachment tab 150 contacts the attachment site where it is to be anchored. The operator then actuates tool 400 to fire the anchor drivers 410 and deploy the anchors (e.g., T-bars) 442 through the attachment tab 150 and attachment structure (e.g., fascia and/or other internal body structure), the drivers 410 are retracted to leave the anchors 442 in place, and sutures 444 are cinched up against the attachment tab and attachment site to anchor device 10 in place via anchors 442, sutures 444 and knots or other fixation mechanism, with the attachment tab 150 and internal body structure attachment site sandwiched between the anchors 442 and knots or other fixation mechanism via sutures 444, FIG. 19P.

Next, a local anesthetic, such as Marcaine, or the like can be delivered to the target implantation site through a lumen in tool 400 as illustrated in FIG. 19Q, such as through a lumen extending through tool 400 adjacent the lumen that endoscope 330 is received in. At FIG. 19R a source of pressurized fluid 560 is next connected to fill tube 12 and fillable member 10em is filled with the fluid, to at least the initial volume.

Tool 400 is decoupled from device 10 and then removed from conduit 600. Conduit 600 and large cannula 310L may also be removed from the patient at this time, as schematically illustrated in FIG. 19S. Fill tube 12, extends proximally out of opening 223, as illustrated in FIG. 19S.

At FIG. 19T, fill tube 12 is cut to the appropriate length to join adjustment member 80 thereto and to reduce any excessive length of fill tube 12 that might otherwise exist. After securing adjustment member 80 to the fascia 127f/abdominal wall 127 to both anchor it as well as to close the opening through the fascia 127f, any adjustment of the volume of expandable member can be performed as needed, and then the patient can be closed, including closing of opening 223 to complete the procedure. As in other embodiments, adjustment member 80 can be installed attached to the abdominal wall 127/fascia 127f at a location other than the opening 223. In such cases, opening 223 is closed around the fill tube 12 extending therefrom, and the adjustment member is attached to the fascia 127f and/or abdominal muscle 127 at another location, so that attachment member 80 does not need to perform the closure function for closing the opening 223.

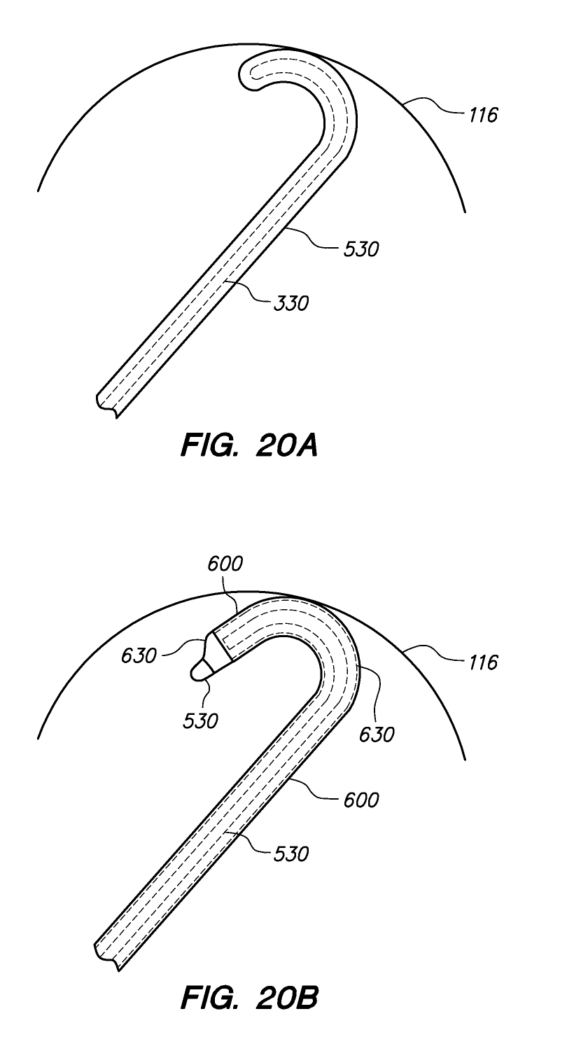

FIG. 20A illustrates a variation of the method described above with regard to FIGS. 19A-19J, wherein a flexible endoscope 330 is inserted into the guide 530, rather than a rigid endoscope. In this case, the endoscope 330 can be extended up and around the curvature of the diaphragm 116, within guide 530 as shown in FIG. 20A, thereby providing direct visualization of the target implantation site.

FIG. 20B illustrates use of a conduit 600 in which a least a distal end portion thereof is flexible, and an obturator 630 in which at least a distal end portion thereof is flexible. In this case, rather than limiting the insertion of the conduit 600 to just approaching near the diaphragm 116 as in FIG. 19H, the flexibility of the conduit 600 and obturator 630 allow them to follow over the distal end curved portion of guide 530 as shown in FIG. 20B. Advantageously, this allow the fillable member 10em to be delivered right to the target location, or even a little beyond the target location, wherein the fillable member can be easily retracted into the desired target location.

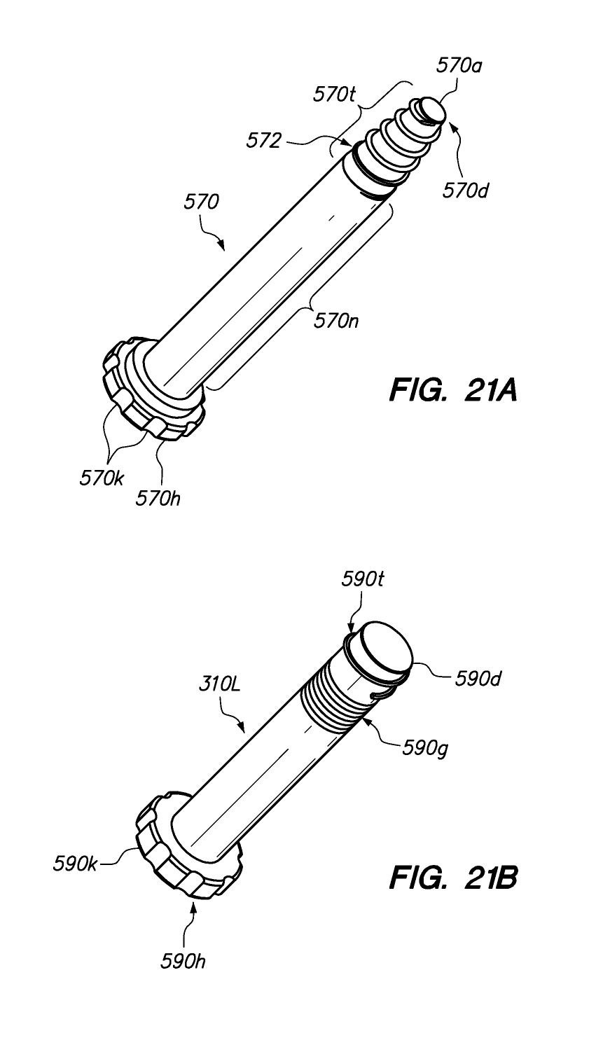

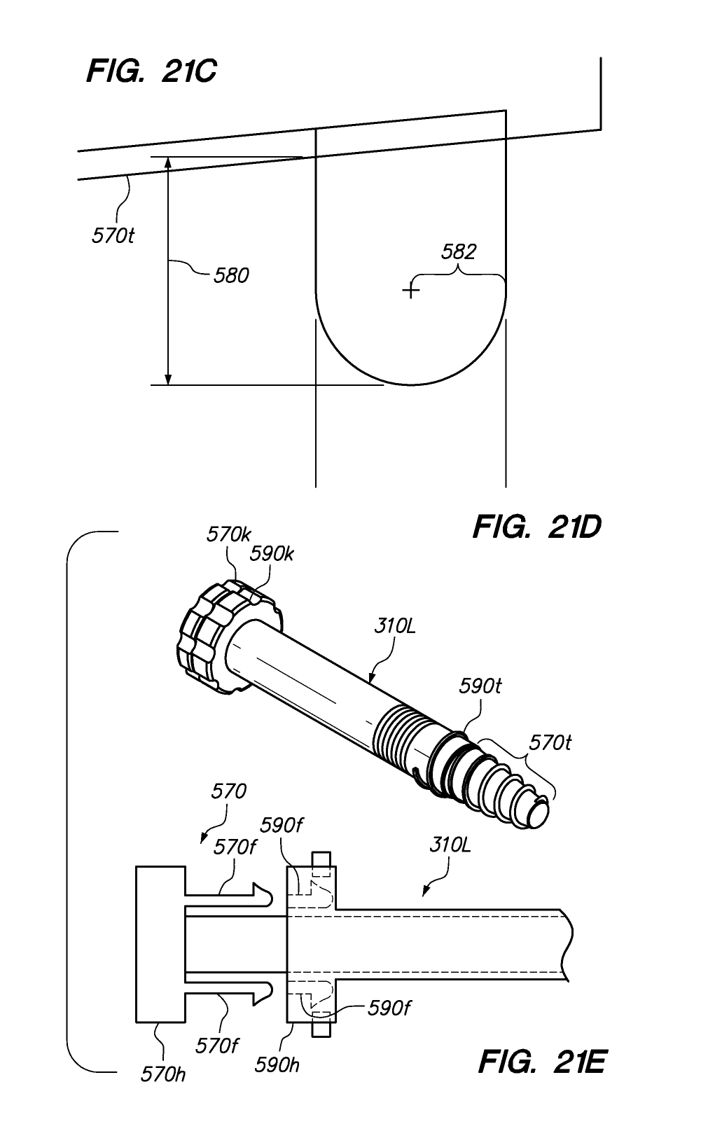

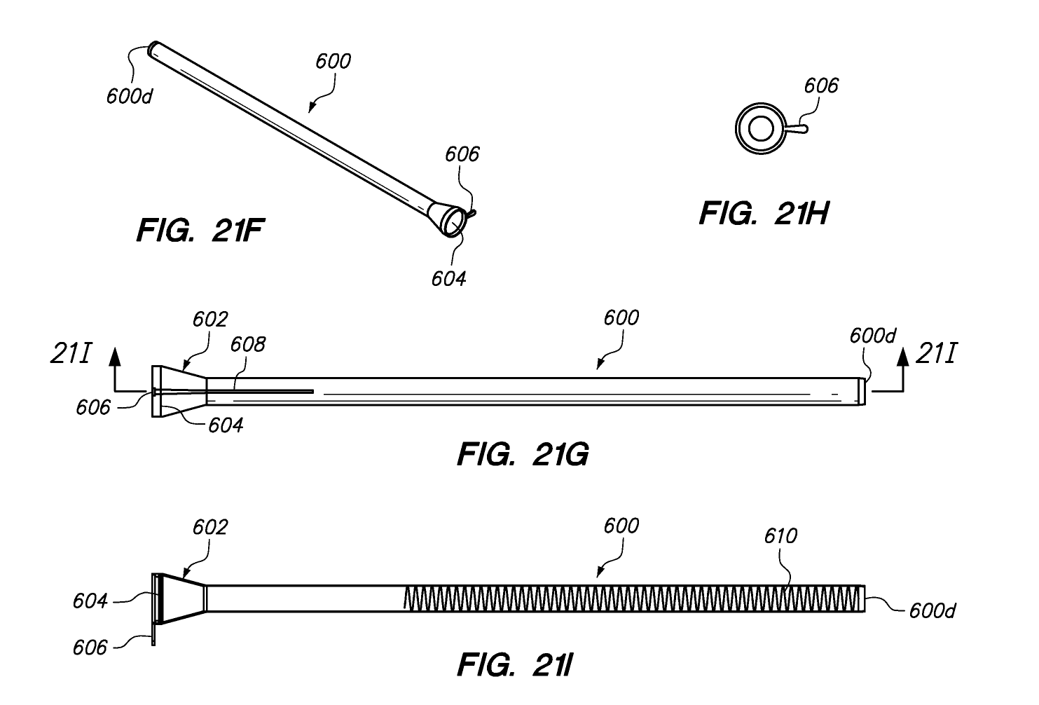

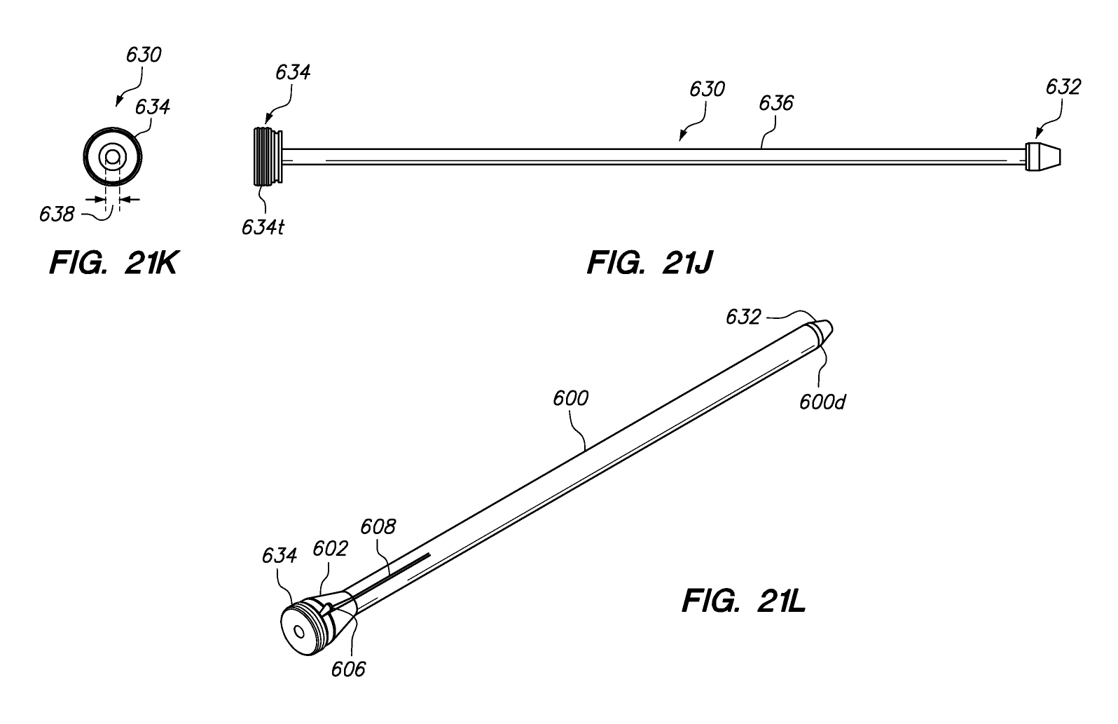

FIGS. 21A-21L show an embodiment and variations of a system comprising tools that can be used in carrying out parts of a procedure for delivering and implanting a device in a manner as described above, particularly in portions of the procedure of FIGS. 19A-19T, although at least some of the tools shown can be used in one or more of the procedures described prior to the procedure of FIGS. 19A-19T. Still further these tools can be used to deliver other implants not described herein. The tools shown and described with regard to FIGS. 21A-21L can be made from one or more of the following materials: polycarbonate, glass-filled polycarbonate, glass-filled nylon, Grilamid® (semi-lubricious nylon product) Grivory® (semi-lubricious nylon product), polyetheretherketone (PEEK), Teflon® (polytetrafluoroethylene) and/or Delrin® (acetal resin) or other injection molded, biocompatible plastic.

FIG. 21A shows an embodiment of a dilator 570 and FIG. 21B shows an embodiment of a large cannula or introducer 310L that can be used with the dilator 570 of FIG. 21A in any of the same manners described above with regard to the dilator 570 and large cannula 310L described with regard to FIGS. 14A-14E, including use for delivery and placement of a conduit 600 like described in FIGS. 19F-19H. Like the embodiment of FIG. 14A, dilator 570 is tapered, with a large threadform 572 along the tapered portion 570t and transitioning to the non-tapered portion 570n. In at least one embodiment the threadform 572 is about 1.5 threads per inch, has a pitch of about 2.67 and the tapered portion has a taper of about eight degrees. In another embodiment, the threadform 572 is about 2.67 threads per inch, has a pitch of about 0.375 and the tapered portion has a taper of about eight degrees. Each of these specifications may vary, but the threadform should remain large (e.g. about 1.1 to about 3.3 threads per inch) and the threads should extend sufficiently from the surface of the taper, e.g., about 0.065″ to about 0.125″, typically about 0.080″, but be blunt (rounded) so as to grab the tissues to drive the dilator into the abdominal cavity as the dilator 570 is rotated, without cutting the tissues that the threadform 572 contacts. FIG. 21C illustrates one specific embodiment of a threadform 572 that extends from the surface of the taper 570t by a distance 580 of about 0.080 inches and wherein the free or exposed edge of the threadform 572 has a radius of curvature 582 of about 0.030″.

Dilator 570 has a central annulus or lumen 570a extending therethrough which has a diameter slightly larger than the outside diameter of guide 530. Accordingly, annulus 570a may have a diameter of about 0.5″ or slightly larger. In one particular embodiment, dilator 570 has an inside diameter of about 0.505″ formed by annulus or lumen 570a, and an outside diameter of the non-tapered portion is about 0.995″, with a length of the overall dilator 570 being about 8.7″. In another particular embodiment, the inside diameter and length were the same, but the outside diameter of the non-tapered portion 570n was about 1.060″. In still another embodiment, the inside diameter is the same, but the length of the dilator 570 is about 16.16″ and the outside diameter of the non-tapered portion 570n is about 1.588″. Thus, the inside diameter of dilator 570 at the distal end 570d closely matches the outside diameter of tube 534 being only slightly larger (e.g., about 0.005″± about 0.002″) to allow free sliding of the dilator 570 over the guide 530, but fitting closely to prevent this interface from grabbing tissues as the dilator 570 is advanced over guide 530. The distal end of dilator 570, where the tapered portion begins has an outside diameter of slightly greater than the annulus diameter, e.g., about 0.6″ to about 0.7″ and tapers to the cross-sectional dimension of the non-tapered section 570n, which may, for example, have an outside diameter of about 0.8 inches to about 1.7 inches.

In FIG. 21A, dilator 570 additionally includes an enlarged handle 570h at a proximal end thereof that is configured to be grasped by a user to facilitate an increase in the amount of torque the user can apply to the dilator 570 by rotating handle 570h. Thus, handle 570h has a larger outside diameter than the non-tapered cylindrical portion 570n of dilator 570. Further, handle 570h can be provided with knurls 570k or other features that render handle 570h less smooth or otherwise increase friction, to prevent the user's hand from slipping during torquing.

The large cannula 310L of FIG. 21B is configured to slide over dilator 570 with a close, but freely sliding fit (e.g., inside diameter of large cannula 310L is about 0.005″±about 0.002″ greater than outside diameter of portion 570n) and large cannula 310L has a length such that when handle 590h contacts handle 570h, the threaded, tapered portion 570t of dilator 570 extends distally of the distal end of large cannula 310L as shown in the assembled view of FIG. 21D. In another embodiment, the close, but freely sliding fit is provided wherein the inside diameter of large cannula 310L is about 0.012″±about 0.005″ greater than outside diameter of portion 570n In one embodiment where the dilator had a length of about 8.67″, and inside diameter of about 0.505″ and the portion 570n had an outside diameter of about 0.995″, the large cannula 310L had a length of about 6.375″, an inside diameter of about 1.055″ and an outside diameter of about 1.105″. In another embodiment where the dilator had a length of about 16.16″, and inside diameter of about 0.505″ and the portion 570n had an outside diameter of about 1.588″, the large cannula 310L had a length of about 11.855″, an inside diameter of about 1.610″ and an outside diameter of about 1.690″. In another particular embodiment the dilator had the a length of about 8.67″ and the same inside diameter as the previous embodiments, but an outside diameter of about 1.060″ and the large cannula had a length of about 6.375″, an inside diameter of about 1.065″ and an outside diameter of about 1.115″. In all embodiments, the inside diameter of large cannula 310L forms a close fit with the outside diameter of the cylindrical portion 570 to allow free sliding between the components, but to prevent snagging of tissue between the distal end of large cannula 310L and dilator 570 as these components are inserted into the body.

Large cannula 310L may be provided with a first threadform 590t that matches the pitch of the threadform 570t and extends from the surface of the cylindrical main body of large cannula 310L by a distance equal or similar to the distance that threads 570t extend from the conical portion of the dilator 570. In this way, threads 590t can be aligned with threads 570t so that the threadform 590t acts as a continuation of threadform 570t by extending smoothly and substantially continuously therefrom as illustrated in FIG. 21D. However, it is not critical that the threads 570t and 590t are aligned in this manner, as threads 590t can start independently of the thread 570t after the thread 570t has passed through the fascia. The large cannula threads can alternatively be a different threadform and pitch. To assist in alignment of the threads 570t, 590t and maintenance of the alignment, handle pattern 590k that both assists grip by the user, and matches up with the pattern 570k on the handle 570h of the dilator. Accordingly, as shown in FIG. 21D, when threads 570t are aligned with threads 590t the knurling pattern 590k aligns with knurling pattern 570k. By maintaining alignment of the patterns 570k, 590k (the user can maintain alignment by grasping both 570k and 590k in his or her hand) during torquing, threads 570t 590t can be seamlessly threaded in through an opening in the fascia, for example.

Alternatively or additionally, handle 570h may be provided with at least one fastening component 570f and handle 590h may be provided with at least one mating fastening components 590f, one for each respective fastening component 570h. As shown in FIG. 21E, handle 570h includes two male fastening components 570h and handle 590h includes two corresponding mating female components 590f. However, one or more than two such components may be provided on handle 570h and, correspondingly, in handle 590h. Further, the male component(s) can be provided on handle 590h and the female components can be provided in handle 570h. Still further, although bayonet couplings 570f and mating female receptacles 590f are shown, alternative mating components may be used, such as shafts with ball and detent arrangements, or any of a number of mating, releasable mechanical fixtures. In any case. The mating mechanical members 570h and 590h, when connected, maintain the large cannula 310L fixed relative to the dilator 570, both in the axial direction, as well as rotationally. Accordingly, these fixtures can be arranged so that when they are connected together, the threads 570t and 590t are aligned, and the distal end of the large cannula 310L is properly axially aligned with the distal end portion of the dilator 570 as intended. A release mechanism 591 may be provided that the user can actuate, once the cannula 310L has been properly positioned so that the distal portion including threads 590t has been threaded through the opening in the fascia, to release the mechanical fixation member 570h, 590h and then the operator can remove the dilator 570 form the large cannula 310L and the patient 1 by withdrawing on handle 570h while holding handle 590h stationary relative to the patient 1. In the example shown in FIG. 21E, the release mechanism 591 comprises a pair of release buttons 591 that the operator can press on to release the bayonet male members 570f from the receptacles 590f. Handles 570h, 590h can have substantially the same size/outside diameter, as shown in FIG. 21D, but this is not necessary.

The distal end 590d of large cannula 310L may be chamfered so that it tapers towards the dilator 570 when assembled thereover, thereby further reducing the risk of snagging tissue (e.g., fascia) as the tools are threaded into the body. Alternatively, the tip 590d may be flexible and tapered to a smaller diameter to create intimate contact and smooth transition with the dilator 570. In this embodiment, the tip 590d could be composed of an elastomeric material or a more rigid material where the tip 590d is radially interrupted to allow the stiffer material to flex radially outwards to allow an interference fit that slides under low force. This same type of transition could be applied to the dilator tip 570d, to provide a smooth transition to the guide tube 530. In addition to aiding in the dilation procedure, threads 590d provide tactile feedback to the user to let the user know when the distal end of large cannula 310L has been threaded into the abdominal cavity through the hole in the fascia, as the user can feel the cannula 310L being drawn in through the hole in the fascia by the threads 590t as the cannula 310L is rotated. Further, the threadforms allow the user to feel when they have passed through the fascial hole such that the large cannula 310 can then translate forward more easily. This tactile feedback allows the user to feel when the end of the large cannula 310 has appropriately passed beyond the fascia. Further, the distal threads 590t on the introducer 310L are configured to help prevent the large cannula 310L from accidentally pulling out of the abdominal cavity. Coarse ridges 590g may be provided on the distal end portion of large cannula 310L proximal of threads 590t. The coarse ridges 590g function to increase friction between them and the surrounding tissues to help prevent movement of the large cannula 310L relative to the patient's body, once it has been inserted in the desired position. As shown, the coarse ridges are parallel to one another and closely spaced. Once the distal end portion of large cannula 310L has been installed through the opening in the fascia, dilator 570 can be withdrawn from the cannula 310L and the patient 1 leaving the large cannula 310L in place to provide access to the abdominal cavity by tools and/or implants. Guide 530 may also be left in place to guide tools and/or implants. Alternatively, guide 530 may be removed to provide greater cross-sectional area of the large cannula 310L, such as for insertion and use of one or more tools and/or insertion and delivery of one or more implants.

FIGS. 21F-21I illustrate various embodiments of conduit 600 that can be inserted through large cannula 310L to extend far into the abdominal cavity, for delivery of one or more tools and/or implants therethrough. Thus, the location that is “far into the abdominal cavity’ refers to the length of the cannula that is required to reach the location that is far into the abdominal cavity. Therefore the location can actually be quite shallow, relative to skin lying directly over it such as a location along the fascia or ribs. However, the location is “far” in the sense that it located away from the opening through the skin by a relatively large distance, a distance that is significantly greater than the length of the large cannula 310L, as noted above. The embodiment of FIG. 21F is formed of relatively rigid plastic. In one embodiment, this relatively rigid conduit 600 had a length of about 28.25 inches, an inside diameter of about 1.00 inches and an outside diameter of about 1.05 inches. In another embodiment, this relatively rigid conduit 600 had a length of about 24.325 inches, an inside diameter of about 1.425 inches and an outside diameter of about 1.05 inches. Conduit 600 may include a chamfered or otherwise tapered distal end 600d so that it tapers towards the obturator 630 when assembled thereover, thereby reducing the risk of snagging tissue as the tools are inserted into the abdominal cavity, and generally helping to keep fluids and other tissues out of the conduit 600 as it is being advanced. Further optionally, the tapered distal end 600d may compress against the distal tip of the obturator 630 and/or form an interference fit therewith, preventing the distal tip of the obturator 630 from passing therethrough so that the obturator 630 be used to push against the conduit 600 via this contact to dive the conduit into the abdominal cavity and prevent the distal end of the conduit 600 from compressing or buckling toward the proximal end of the conduit 600. This fit between the distal end 600d and distal tip of the obturator 630 can also effectively seal the contact between the tapered distal end 600d and the distal end part/distal tip of the obturator 630, thereby preventing fluid inflow and tissue ingress into conduit 600 as it is advanced.

As noted above, a flared or funnel portion 602 may be provided, either integrally with or attached to the proximal end portion of conduit 600. A seal 604 such as an o-ring may be provided to seat the proximal end portion of the obturator 630 or proximal end of a tool. Further, a grasping tab 606 provided that can be pulled by the user to remove a perforated strip from the funnel portion 602 to expose slot 608. In instances where funnel portion 602 and the proximal end portion of conduit 600 are flexible, this allows deformation of the funnel portion 602 and proximal end portion of the conduit along slot 208 to allow a shaft, handle or tube that extends transversely from a tool (e.g., light post of an endoscope, handle 412t of tool 400, etc.) to slide therealong, thereby reducing the effective length of the tool 400 endoscope 330 or other tool that needs to be provide to permit a distal end thereof extend distally of the conduit 600. This is particularly useful for the part of the procedure illustrated in FIGS. 19K-19L, as it allows a relatively shorter tool 400 and endoscope 330 to be used while still allowing conduit 600 to be retracted sufficiently to fully expose expandable member 10em. In embodiments where funnel portion (and optionally, the proximal end portion of conduit 600) are rigid, the funnel portion 602 and adjoining proximal end portion of conduit 600 can be provided as half pieces that are hinged together, wherein a pair of opposing separations are formed between the halves (one in the location of and replacing slot 608 and one at a location 180 degrees from there) to allow separation of the funnel portion 602 and proximal end portion prior to retracting the conduit 600 off of expandable member 10em.

FIGS. 21G-21I illustrate another embodiment of a conduit 600 in which at least a distal end portion thereof is flexible. In this embodiment, the main tube of the conduit is formed of an elastomer, such as silicone, and a coil 610, such as a stainless steel coil, Nitinol coil, or the like, is encapsulated in the elastomer along at least the distal end portion of the conduit 600. Note that the chamfered or tapered distal end 600d is not reinforced with the coil 610. At least a 4″ length of the conduit 600 extending proximally from the unreinforced distal end 600d is reinforced with coil 610. In other embodiments, a least a quarter or at least a third or at least half of the length of the conduit 600 extending proximally from the unreinforced distal end 600d is reinforced with coil 610. In the example shown in FIG. 21G and the sectional view of FIG. 21I, coil 610 reinforces more than half of the entire length to the main body tube of conduit 600, extending proximally from the unreinforced distal end 600d. In still other embodiments, coil 610 may extend proximally from unreinforced distal end 600d and support the entire length of the tube up to the distal end of slot 608. In embodiments where slot 608 is not present, coil 610 may reinforce the entire length of the tube of conduit 600, but typically not the tapered distal end 600d or funnel portion 602. Portions of the main tube of conduit 600 that are proximal of the proximal end of coil 610 may be made of an alternative material, such as a rigid polymer, so that this portion of the conduit is not flexible. Alternatively, portions of the main body of conduit 600 that are proximal of the proximal end of coil 610 may be flexible. Further alternatively, the main body of the conduit 600 can have no coil reinforcement 610, but instead have reinforcements running longitudinally to allow bending but prevent stretching or buckling.

The reinforcement provided by coil 610 helps preserve the substantially circular cross section of the conduit 600 as it bends along a portion supported by coil 610 and coil 610 serves to prevent kinking along a supported portion as it is bent. In one particular embodiment a conduit of the type described with regard to FIGS. 21G-21I had a length of about 28.25 inches, an inside diameter of about 1.00 inch and an outside diameter of about 1.060 inches. In another embodiment, a conduit of the type described with regard to FIGS. 21G-21I had a length of about 24.325 inches, an inside diameter of about 1.425 inches and an outside diameter of about 1.505 inches.

FIGS. 21J-21K illustrate a plan view and a proximal end view of an obturator 630 that is configured to be placed in conduit 600 and used to deliver conduit 600 through large cannula 310L and over guide 530 to deliver a distal end portion of conduit 600 far into the abdominal cavity. Obturator 630 has a length slightly greater than the length of conduit 600 so that when the tapered portion of distal tip 632 contacts chamfered end 600d, the handle 634 at the proximal end of obturator 630 extends slightly proximally of the proximal end of conduit 600 or the proximal end of funnel portion 602 when provided at the proximal end of conduit 600. Handle 634 and distal tip 632 are typically rigid and may be injection molded from hard plastic. Shaft 636 is relatively flexible and may be formed of extruded PEBAX® (polyether bock amides) or similar lubricious polymer extrusion that facilitates it sliding over guide 530, or may have a corrugated geometry or an interrupted linked geometry to allow flexibility.

A textured surface 634t such as grooves or the like may be provided on handle 634 to enhance grip by a user, as well as interfacing with seal 604. In one particular embodiment obturator 630 had an overall length of about 29.64″, an inside diameter 638 (see proximal end view of FIG. 21K) of about 0.505″, an outside diameter of shaft 636 of about 0.565″, an outside diameter of distal tip 632 of about 0.995″ and an outside diameter of handle of about 1.880″. In another particular embodiment, obturator 630 had an overall length of about 26.307″, an inside diameter 638 (see proximal end view of FIG. 21K) of about 0.505″, an outside diameter of shaft 636 of about 0.565″, an outside diameter of distal tip 632 of about 1.375″ and an outside diameter of handle of about 1.950″.

FIG. 21L illustrates obturator 632 having been inserted into conduit 600. Preferably, the contact between obturator 632 and conduit 600 occurs only between the distal tip 632 (tapered portion) and the chamfered end 600d, and between the funnel portion 602/seal 604 and the handle 634. This maximizes the ability of conduit 600 to make bends of the smallest possible bend radii, without kinking or distortion.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the present invention as recited in the following claims.

That which is claimed is:

1. A method of treating a patient comprising:
inserting a guide member through an opening in the patient
establishing a delivery tract extending from the opening into the abdominal cavity, and advancing the guide member between the fascia and the abdominal organs of the patient;
positioning a distal end portion of the guide member at a target location within the boundaries of the rib cage;
delivering a conduit over the guide member;
removing the guide member, while leaving the conduit in position, directed toward the target location;
advancing an implantable device to a distal end of the conduit;
retracting the conduit to expose the implantable device;
securing the implantable device to at least one internal body structure at the target location;
at least partially filling a fillable member of the implantable device with fluid;
attaching an adjustment member to a fill tube in fluid communication with the fillable member and anchoring the adjustment member to an internal body structure; and
closing the opening.

2. The method of claim 1, further comprising viewing at least one of the fascia and bowel with an endoscope inserted into the guide member.

3. The method of claim 1, wherein said advancing an implantable device to a distal end of the conduit comprises inserting a delivery and attachment tool into the conduit, wherein the delivery and attachment tool has the implantable device mounted to a distal end portion thereof, said implantable device in a compact configuration, and an endoscope received in said tool and being configured to view locations along the distal end portion of said tool.

4. The method of claim 1, wherein said advancing an implantable device to a distal end of the conduit comprises inserting a delivery and attachment tool into the conduit, wherein the delivery and attachment tool has the implantable device mounted to a distal end portion thereof, delivering the implantable device out of the distal end of the conduit and inserting an endoscope into the conduit alongside the tool and delivering a distal end of the endoscope out of the distal end of the conduit.

5. The method of claim 1, wherein an endoscope is inserted in said guide member; said advancing an implantable device to a distal end of the conduit comprises inserting a delivery and attachment tool into the conduit, wherein the delivery and attachment tool has the implantable device mounted to a distal end portion thereof; and wherein the guide member and endoscope are inserted into the conduit alongside the delivery and attachment tool after the implantable device has been delivered out of the distal end of the conduit and the distal end of the guide member and endoscope are delivered out of the distal end of the conduit.

6. The method of claim 5, further comprising:
visually confirming, via the endoscope, that a location between an attachment tab of the implantable device and the target location to which the device is to be attached is clear of bowel.

7. The method of claim 6, further comprising: after said visually confirming, actuating the tool to deliver at least one anchor into the target location.

8. The method of claim 7, further comprising:
securing the attachment tab to the target location where the at least one anchor has been delivered.

9. The method of claim 8, further comprising:
detaching the tool from the implantable device and removing the tool and conduit from the patient.

10. The method of claim 1, wherein the guide member has a flexible distal end portion, and said flexible distal end portion is guided by a patient's anatomy to curve around a curved structure in the patient's anatomy.

11. The method of claim 10, wherein the conduit has a flexible distal end portion and said conduit is advanced over the curved distal end portion of said guide member, said distal end portion of said conduit curving as it is guided by the curved distal end portion of said guide member.

12. The method of claim 7, wherein said actuating the tool drives a plurality of anchor drivers through at least one internal body structure.

13. The method of claim 12, needles drive through the at least one internal body structure, said needles carrying anchors.

14. The method of claim 13, wherein said needles are retracted after being driven through the at least one internal body structure, and wherein said anchors self extract from said needles, are anchored and are not retracted back through the at least one body structure.

15. The method of claim 8, wherein the tool automatically cinches the attachment tab up against the at least one internal body structure that the implantable device is attached to.

16. The method of claim 15, further comprising cutting by said tool of excess suture material extending proximally from the attachment tab.

17. The method of claim 1, further comprising dilating an opening in the fascia leading into the abdominal cavity along the delivery tract, prior to said delivering a conduit over the guide member.

18. The method of claim 17, further comprising using a dilator and introducer to perform said dilating, and removing said dilator after said dilating, while maintaining said introducer through the dilated opening, wherein said conduit is delivered through the introducer over the guide.
